SEQUENCE LISTING (39 sequences)

SEQ ID NO: 1 - RNA, 5959 nt, Homo sapiens
gaauuccggaugcucagaugaaagccccgagaucacagagacccggcgagaucacagagacccggccugaaggaacguggaaagaccaauguaccuguuuugaccgguugccuggagcaagaaguuccaguuggggagaauuuucagaagauaaagucggagauuguggaaagacuugacuugcagcauuacucuacugacuggcagagacaggagagguagaugucaacgcccacagacccuggugcgaugccccacccagggccuucgccggguccugggccuuccccugggccaauucuugggccuaguccaggaccaggaccauccccagguuccguccacagcaugauggggccaaguccuggaccuccaagugucucccauccuaugccgacgaugggguccacagacuucccacaggaaggcaugcaucaaaugcauaagcccaucgaugguauacaugacaaggggauuguagaagacauccauuguggauccaugaagggcacugguaugcgaccaccucacccaggcaugggcccuccccagaguccaauggaucaacacagccaagguuauaugucaccacacccaucuccauuaggagccccagagcacgucuccagcccuaugucuggaggaggcccaacuccaccucagaugccaccaagccagccgggggcccucaucccaggugauccgcaggccaugagccagcccaacagaggucccucaccuuucaguccuguccagcugcaucagcuucgagcucagauuuuagcuuauaaaaugcuggcccgaggccagccccuccccgaaacgcugcagcuugcaguccaggggaaaaggacguugccuggcuugcagcaacaacagcagcagcaacagcagcagcagcagcagcagcagcagcagcagcagcagcaacagcagccgccgcaaccacagacgcagcaacaacagcagccggcccuuguuaacuacaacagaccaucuggcccggggccggagcugagcggcccgagcaccccgcagaagcugccggugcccgcgcccggcggccggcccucgcccgcgccccccgcagccgcgcagccgcccgcggccgcagugcccgggcccucagugccgcagccggccccggggcagcccucgcccguccuccagcugcagcagaagcagagccgcaucagccccauccagaaaccgcaaggccuggaccccguggaaauucugcaagagcgggaauacagacuucaggcccgcauagcucauaggauacaagaacuggaaaaucugccuggcucuuugccaccagauuuaagaaccaaagcaaccguggaacuaaaagcacuucgguuacucaauuuccagcgucagcugagacaggaggugguggccugcaugcgcagggacacgacccuggagacggcucucaacuccaaagcauacaaacggagcaagcgccagacucugagagaagcucgcaugaccgagaagcuggagaagcagcagaagauugagcaggagaggaaacgccgucagaaacaccaggaauaccugaacaguauuuugcaacaugcaaaagauuuuaaggaauaucaucggucuguggccggaaagauccagaagcucuccaaagcaguugcaacuuggcaugccaacacugaaagagagcagaagaaggagacagagcggauugaaaaggagagaaugcggcgacugauggcugaagaugaggaggguuauagaaaacugauugaucaaaagaaagacaggcguuuagcuuaccuuuugcagcagaccgaugaguauguagccaaucugaccaaucugguuugggagcacaagcaagcccaggcagccaaagagaagaagaagaggaggaggaggaagaagaaggcugaggagaaugcagaggguggggagucugcccugggaccggauggagagcccauagaugagagcagccagaugagugaccucccugucaaagugacucacacagaaaccggcaagguucuguucggaccagaagcacccaaagcaagucagcuggacgccuggcuggaaaugaauccugguuaugaaguugccccuagaucugacagugaagagagugauucugauuaugaggaagaggaugaggaagaagaguccaguaggcaggaaaccgaagagaaaauacuccuggauccaaauagcgaagaaguuucugagaaggaugcuaagcagaucauugagacagcuaagcaagacguggaugaugaauacagcaugcaguacagugccaggggcucccaguccuacuacaccguggcucaugccaucucggaguggguggagaaacagucugcccuccuaauuaaugggacccuaaagcauuaccagcuccagggccuggaauggaugguuucccuguauaauaacaacuugaacggaaucuuagccgaugaaauggggcuuggaaagaccauacagaccauugcacucaucacuuaucugauggagcacaaaagacucaauggccccuaucucaucauuguuccccuuucgacucuaucuaacuggacauaugaauuugacaaaugggcuccuucuguggugaagauuucuuacaaggguacuccugccaugcgucgcucccuugucccccagcuacggaguggcaaauucaauguccucuugacuacuuaugaguauauuauaaaagacaagcacauucuugcaaagauucgguggaaauacaugauaguggacgaaggccaccgaaugaagaaucaccacugcaagcugacucaggucuugaacacucacuauguggcccccagaaggauccucuugacugggaccccgcugcagaauaagcucccugaacucugggcccuccucaacuuccuccucccaacaauuuuuaagagcugcagcacauuugaacaaugguucaaugcuccauuugccaugacuggugaaaggguggacuuaaaugaagaagaaacuauauugaucaucaggcgucuacauaagguguuaagaccauuuuuacuaaggagacugaagaaagaaguugaaucccagcuucccgaaaaaguggaauaugugaucaagugugacaugucagcucugcagaagauucuguaucgccauaugcaagccaaggggauccuucucacagaugguucugagaaagauaagaaggggaaaggaggugcuaagacacuuaugaacacuauuaugcaguugagaaaaaucugcaaccacccauauauguuucagcacauugaggaauccuuugcugaacaccuaggcuauucaaauggggucaucaauggggcugaacuguaucgggccucagggaaguuugagcugcuugaucguauucugccaaaauugagagcgacuaaucaccgagugcugcuuuucugccagaugacaucucucaugaccaucauggaggauuauuuugcuuuucggaacuuccuuuaccuacgccuugauggcaccaccaagucugaagaucgugcugcuuugcugaagaaauucaaugaaccuggaucccaguauuucauuuucuugcugagcacaagagcugguggccugggcuuaaaucuucaggcagcugauacaguggucaucuuugacagcgacuggaauccucaucaggaucugcaggcccaagaccgagcucaccgcaucgggcagcagaacgagguccggguacugaggcucuguaccgugaacagcguggaggaaaagauccucgcggccgcaaaauacaagcugaacguggaucagaaagugauccaggcgggcauguuugaccaaaagucuucaagccacgagcggagggcauuccugcaggccaucuuggagcaugaagaggaaaaugaggaagaagaugaaguaccggacgaugagacucugaaccaaaugauugcucgacgagaagaagaauuugaccuuuuuaugcggauggacauggaccggcggagggaagaugcccggaacccgaaacggaagccccguuuaauggaggaggaugagcugcccuccuggaucauuaaggaugacgcugaaguagaaaggcucaccugugaagaagaggaggagaaaauauuugggagggggucccgccagcgccgugacguggacuacagugacgcccucacggagaagcaguggcuaagggccaucgaagacggcaauuuggaggaaauggaagaggaaguacggcuuaagaagcgaaaaagacgaagaaauguggauaaagauccugcaaaagaagauguggaaaaagcuaagaagagaagaggccgcccucccgcugagaaacugucaccaaauccccccaaacugacaaagcagaugaacgcuaucaucgauacguguauaaacuacaaagauaguuguaacguggagaaggugcccaguaauucucaguuggaaauagaaggaaacaguucagggcgacagcucagugaagucuucauucaguuaccuucaaggaaagaauuaccagaauacuaugaauuaauuaggaagccaguggauuucaaaaaaauaaaggaaaggauucguaaucauaaguaccggagccuaggcgaccuggagaaggaugucaugcuucucugucacaacgcucagacguucaaccuggagggaucccagaucuaugaagacuccaucgucuuacagucaguguuuaagagugcccggcagaaaauugccaaagaggaagagagugaggaugaaagcaaugaagaggaggaagaggaagaugaagaagagucagaguccgaggcaaaaucagucaaggugaaaauuaagcucaauaaaaaagaugacaaaggccgggacaaagggaaaggcaagaaaaggccaaaucgaggaaaagccaaaccuguagugagcgauuuugacagcgaugaggagcaggaugaacgugaacagucagaaggaagugggacggaugaugagugaucaguauggaccuuuuuccuugguagaacugaauuccuuccuccccugucucauuucuacccagugaguucauuugucauauaggcacuggguuguuucuauaucaucaucgucuauaaacuagcuuuaggauagugccagacaaacauaugauaucaugguguaaaaaacacacacauacacaaauauuuguaacauauugugaccaaaugggccucaaagauucagauugaaacaaacaaaaagcuuuugauggaaaauauguggguggauaguauauuucuaugggugggucuaauuugguaacgguuugauugugccugguuuuaucaccuguucagaugagaagauuuuugucuuuuguagcacugauaaccaggagaagccauuaaaagccacugguuauuuuauuuuucaucaggcaauuuucgagguuuuuauuuguucgguauuguuuuuuuacacugugguacauauaagcaacuuuaauaggugauaaauguacaguaguuagauuucaccugcauauacguuuuuccauuuuaugcucuaugaucugaacaaaagcuuuuugaauuguauaagauuuaugucuacuguaaacauugcuuaauuuuuuugcucuugauuuaaaaaaaaguuuuguugaaagcgcuauugaauauugcaaucuauauaguguauuggauggcuucuuuugucacccugaucuccuauguuaccaauguguaucgucuccuucucccuaaaguguacuuaaucuuugcuuucuuugcacaaugucuuugguugcaagucauaagccugaggcaaauaaauuccaguaauuucgaagaaugugguguuggugcuuuccuaauaaagaaauaauuucgcuugaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaggaauuc

SEQ ID NO: 2 - RNA, 5779 nt, Homo sapiens
ggagaggccgccgcggugcugagggggaggggagccggcgagcgcgcgcgcagcgggggcgcggguggcgcgcgugugugugaagggggggcgguggccgaggcgggcgggcgcgcgcgcgaggcuuccccucguuuggcggcggcggcggcuucuuuguuucgugaagagaagcgagacgcccauucugcccccggccccgcgcggaggggcgggggaggcgccgggaagucgacggcgccggcggcuccugcgucucgcccuuuugcccaggcuagagugcaguggugcggucaugguucacugcagccucaaccuccuggacucagcaggaggccacugucugcagcucccgugaagauguccacuccagacccaccccugggcggaacuccucggccagguccuuccccgggcccuggcccuuccccuggagccaugcugggcccuagcccgggucccucgccgggcuccgcccacagcaugauggggcccagcccagggccgcccucagcaggacaccccauccccacccaggggccuggaggguacccucaggacaacaugcaccagaugcacaagcccauggaguccaugcaugagaagggcaugucggacgacccgcgcuacaaccagaugaaaggaauggggaugcggucagggggccaugcugggauggggcccccgcccagccccauggaccagcacucccaagguuaccccucgccccuggguggcucugagcaugccucuaguccaguuccagccaguggcccgucuucggggccccagaugucuuccgggccaggaggugccccgcuggauggugcugacccccaggccuuggggcagcagaaccggggcccaaccccauuuaaccagaaccagcugcaccagcucagagcucagaucauggccuacaagaugcuggccagggggcagccccuccccgaccaccugcagauggcggugcagggcaagcggccgaugcccgggaugcagcagcagaugccaacgcuaccuccacccucgguguccgcaacaggacccggcccuggcccuggcccuggccccggcccgggucccggcccggcaccuccaaauuacagcaggccucaugguaugggagggcccaacaugccucccccaggacccucgggcgugccccccgggaugccaggccagccuccuggagggccucccaagcccuggccugaaggacccauggcgaaugcugcugcccccacgagcaccccucagaagcugauucccccgcagccaacgggccgcccuucccccgcgcccccugccgucccacccgccgccucgcccgugaugccaccgcagacccagucccccgggcagccggcccagcccgcgcccauggugccacugcaccagaagcagagccgcaucacccccauccagaagccgcggggccucgacccuguggagauccugcaggagcgcgaguacaggcugcaggcucgcaucgcacaccgaauucaggaacuugaaaaccuucccgggucccuggccggggauuugcgaaccaaagcgaccauugagcucaaggcccucaggcugcugaacuuccagaggcagcugcgccaggaggugguggugugcaugcggagggacacagcgcuggagacagcccucaaugcuaaggccuacaagcgcagcaagcgccagucccugcgcgaggcccgcaucacugagaagcuggagaagcagcagaagaucgagcaggagcgcaagcgccggcagaagcaccaggaauaccucaauagcauucuccagcaugccaaggauuucaaggaauaucacagauccgucacaggcaaaauccagaagcugaccaaggcaguggccacguaccaugccaacacggagcgggagcagaagaaagagaacgagcggaucgagaaggagcgcaugcggaggcucauggcugaagaugaggagggguaccgcaagcucaucgaccagaagaaggacaagcgccuggccuaccucuugcagcagacagacgaguacguggcuaaccucacggagcuggugcggcagcacaaggcugcccaggucgccaaggagaaaaagaagaaaaagaaaaagaagaaggcagaaaaugcagaaggacagacgccugccauugggccggauggcgagccucuggacgagaccagccagaugagcgaccucccggugaaggugauccacguggagagugggaagauccucacaggcacagaugcccccaaagccgggcagcuggaggccuggcucgagaugaacccgggguaugaaguagcuccgaggucugauagugaagaaaguggcucagaagaagaggaagaggaggaggaggaagagcagccgcaggcagcacagccucccacccugcccguggaggagaagaagaagauuccagauccagacagcgaugacgucucugagguggacgcgcggcacaucauugagaaugccaagcaagaugucgaugaugaauauggcgugucccaggcccuugcacguggccugcaguccuacuaugccguggcccaugcugucacugagagaguggacaagcagucagcgcuuauggucaaugguguccucaaacaguaccagaucaaagguuuggaguggcuggugucccuguacaacaacaaccugaacggcauccuggccgacgagaugggccuggggaagaccauccagaccaucgcgcucaucacguaccucauggagcacaaacgcaucaaugggcccuuccucaucaucgugccucucucaacgcuguccaacugggcguacgaguuugacaagugggcccccuccguggugaaggugucuuacaagggauccccagcagcaagacgggccuuugucccccagcuccggagugggaaguucaacgucuugcugacgacguacgaguacaucaucaaagacaagcacauccucgccaagauccguuggaaguacaugauuguggacgaaggucaccgcaugaagaaccaccacugcaagcugacgcaggugcucaacacgcacuauguggcaccccgccgccugcugcugacgggcacaccgcugcagaacaagcuucccgagcucugggcgcugcucaacuuccugcugcccaccaucuucaagagcugcagcaccuucgagcagugguuuaacgcacccuuugccaugaccggggaaaagguggaccugaaugaggaggaaaccauucucaucauccggcgucuccacaaagugcugcggcccuucuugcuccgacgacucaagaaggaagucgaggcccaguugcccgaaaagguggaguacgucaucaagugcgacaugucugcgcugcagcgagugcucuaccgccacaugcaggccaagggcgugcugcugacugauggcuccgagaaggacaagaagggcaaaggcggcaccaagacccugaugaacaccaucaugcagcugcggaagaucugcaaccaccccuacauguuccagcacaucgaggaguccuuuuccgagcacuugggguucacuggcggcauuguccaagggcuggaccuguaccgagccucggguaaauuugagcuucuugauagaauucuucccaaacuccgagcaaccaaccacaaagugcugcuguucugccaaaugaccucccucaugaccaucauggaagauuacuuugcguaucgcggcuuuaaauaccucaggcuugauggaaccacgaaggcggaggaccggggcaugcugcugaaaaccuucaacgagcccggcucugaguacuucaucuuccugcucagcacccgggcuggggggcucggccugaaccuccagucggcagacacugugaucauuuuugacagcgacuggaauccucaccaggaccugcaagcgcaggaccgagcccaccgcaucgggcagcagaacgaggugcgugugcuccgccucugcaccgucaacagcguggaggagaagauccuagcugcagccaaguacaagcucaacguggaccagaaggugauccaggccggcauguucgaccagaaguccuccagccaugagcggcgcgccuuccugcaggccauccuggagcacgaggagcaggaugagagcagacacugcagcacgggcagcggcagugccagcuucgcccacacugccccuccgccagcgggcgucaaccccgacuuggaggagccaccucuaaaggaggaagacgaggugcccgacgacgagaccgucaaccagaugaucgcccggcacgaggaggaguuugaucuguucaugcgcauggaccuggaccgcaggcgcgaggaggcccgcaaccccaagcggaagccgcgccucauggaggaggacgagcuccccucguggaucaucaaggacgacgcggagguggagcggcugaccugugaggaggaggaggagaagauguucggccguggcucccgccaccgcaaggagguggacuacagcgacucacugacggagaagcaguggcucaaggccaucgaggagggcacgcuggaggagaucgaagaggagguccggcagaagaaaucaucacggaagcgcaagcgagacagcgacgccggcuccuccaccccgaccaccagcacccgcagccgcgacaaggacgacgagagcaagaagcagaagaagcgcgggcggccgccugccgagaaacucuccccuaacccacccaaccucaccaagaagaugaagaagauuguggaugccgugaucaaguacaaggacagcagcaguggacgucagcucagcgaggucuucauccagcugcccucgcgaaaggagcugcccgaguacuacgagcucauccgcaagcccguggacuucaagaagauaaaggagcgcauucgcaaccacaaguaccgcagccucaacgaccuagagaaggacgucaugcuccugugccagaacgcacagaccuucaaccuggagggcucccugaucuaugaagacuccaucgucuugcagucggucuucaccagcgugcggcagaaaaucgagaaggaggaugacagugaaggcgaggagagugaggaggaggaagagggcgaggaggaaggcuccgaauccgaaucucgguccgucaaagugaagaucaagcuuggccggaaggagaaggcacaggaccggcugaagggcggccggcggcggccgagccgagggucccgagccaagccggucgugagugacgaugacagugaggaggaacaagaggaggaccgcucaggaaguggcagcgaagaagacugagccccgacauuccagucucgaccccgagccccucguuccagagcugagauggcauaggccuuagcaguaacggguagcagcagauguaguuucagacuuggaguaaaacuguauaaacaaaagaaucuuccauauuuauacagcagagaagcuguaggacuguuugugacuggcccuguccuggcaucaguagcaucuguaacagcauuaacugucuuaaagagagagagagagaauuccgaauuggggaacacacgauaccuguuuuucuuuuccguugcuggcaguacuguugcgccgcaguuuggagucacuguaguuaaguguggaugcaugugcgucaccguccacuccuccuacuguauuuuauuggacaggucagacucgccgggggcccggcgaggguaugucagugucacuggaugucaaacaguaauaaauuaaaccaacaacaaaacgcacagccaaaaaaaaa

SEQ ID NO: 3 - RNA, 1857 nt, Homo sapiens
gccccggccccgccccagcccuccugaucccucgcagcccggcuccggccgcccgccucugccgccgcaaugaugaugauggcgcugagcaagaccuucgggcagaagcccgugaaguuccagcuggaggacgacggcgaguucuacaugaucggcuccgaggugggaaacuaccuccguauguuccgagguucucuguacaagagauaccccucacucuggaggcgacuagccacuguggaagagaggaagaaaauaguugcaucgucacaugguaaaaaaacaaaaccuaacacuaaggaucacggauacacgacucuagccaccagugugacccuguuaaaagccucggaaguggaagagauucuggauggcaacgaugagaaguacaaggcuguguccaucagcacagagccccccaccuaccucagggaacagaaggccaagaggaacagccaguggguacccacccuguccaacagcucccaccacuuagaugccgugccaugcuccacaaccaucaacaggaaccgcaugggccgagacaagaagagaaccuucccccuuugcuuugaugaccaugacccagcugugauccaugagaacgcaucucagcccgaggugcugguccccauccggcuggacauggagaucgaugggcagaagcugcgagacgccuucaccuggaacaugaaugagaaguugaugacgccugagauguuuucagaaauccucugugacgaucuggauuugaacccgcugacguuugugccagccaucgccucugccaucagacagcagaucgaguccuaccccacggacagcauccuggaggaccagucagaccagcgcgucaucaucaagcugaacauccaugugggaaacauuucccugguggaccaguuugagugggacaugucagagaaggagaacucaccagagaaguuugcccugaagcugugcucggagcugggguugggcggggaguuugucaccaccaucgcauacagcauccggggacagcugagcuggcaucagaagaccuacgccuucagcgagaacccucugcccacaguggagauugccauccggaacacgggcgaugcggaccaguggugcccacugcuggagacucugacagacgcugagauggagaagaagauccgcgaccaggacaggaacacgaggcggaugaggcgucuugccaacacgggcccggccugguaaccagcccaucagcacacggcucccacggagcaucucagaagauugggccgccucuccuccaucuucuggcaaggacagaggcgaggggacagcccagcgccauccugaggaucggguggggguggagugggggcuuccagguggcccuucccgguacacauuccauuuguugagccccaguccugccccccaccccacccucccuaccccuccccagucucuggggucaggaagaaaccuuauuuuagguuguguuuuguuuuuguauaggagccccaggcagggcuaguaacaguuuuuaaauaaaaggcaacaggucauguucaauuucuuaaaucuagugucuuuauuucuucuguuacaauaguguugcuuguguaagcagguuagagugcacaguguccccaauuguuccuggcacugcaaaaccaaauuaaacaaucccacaaagaauucugacaucaauguguuuuccucagucaggucuauuucaagauucuagaaguuccuuuuguaaaacuugccuuuaaaacucuuccuccuaaugccaucagaucucuuaacauuggcucacugugggaucuuuccucuuagguugaauuucuacgugaauaucaaagugccuuuuuc

SEQ ID NO: 4 - RNA, 5190 nt, Homo sapiens
ggaauucccgcgaggccggggugggccaggcuguggggacgacgggcugcgacgauggccgcagcggcgggcggcggcgggccggggacagcgguaggcgccacgggcuucggggauucggcggcagccgcaggccuagcuguuuaucgacggaaggaugggggcccggccaccaaguuuugggagagcccggagacggugucccagcuggauucggugcgggucuggcugggcaagcacuacaagaaguauguucaugcggaugcuccuaccaauaaaacacuggcugggcugguggugcagcuucuucaguuccaggaagaugccuuugggaagcaugucaccaacccggccuucaccaaacucccugcaaaguguuucauggauuucaaagcuggaggcgccuuaugucacauucuuggggcugcuuacaaguauaaaaaugaacagggauggcggagguuugaccuacagaacccaucucgaauggaucguaauguggaaauguuuaugaacauugaaaaaacauuggugcagaacaauuguuugaccagacccaacaucuaccucauuccagacauugaucugaaguuggcuaacaaauugaaagauaucaucaaacgacaucagggaacauuuacggaugagaagucaaaagcuucccaccacauuuacccauauucuuccucacaagacgaugaagaaugguugagaccggugaugagaaaagagaagcaaguguuagugcauuggggcuuuuacccagacagcuaugauacuuggguccauaguaaugauguugaugcugaaauugaagauccaccaauuccagaaaaaccauggaagguucaugugaaauggauuuuggacacugauauuuucaaugaauggaugaaugaggaggauuaugagguggaugaaaauaggaagccugugaguuuucgucagcggauuucaaccaagaaugaagagccagucagaaguccagaaagaagagauagaaaagcaucagcuaaugcucgaaagaggaaacauucgccuucgccucccccuccgacaccaacagaaucacggaagaagagugggaagaaaggccaagcuagccuuuaugggaagcgcagaagucagaaagaggaagaugagcaagaagaucuaaccaaggauauggaagacccaacaccuguacccaauauagaagaaguaguacuucccaaaaaugugaaccuaaagaaagauagugaaaauacaccuguuaaaggaggaacuguagcggaucuagaugagcaggaugaagaaacagucacagcaggaggaaaggaagaugaagauccugccaaaggugaucagagucgaucaguugaccuuggggaagauaaugugacagagcagaccaaucacauuauuauuccuaguuaugcaucaugguuugauuauaacuguauucaugugauugaacggcgugcucuuccugaguucuucaauggaaaaaacaaauccaagacuccagaaauauacuuggcauaucgaaauuuuaugauugacagcuaucgucuaaacccccaagaguauuuaacuagcacugcuugucggaggaacuugacuggagaugugugugcugugaugaggguccaugccgggggagagcaguggggacucguuaauuaccaaguugacccggaaaguagacccauggcaaugggaccuccuccuacuccucauuuuaauguauuagcugauaccccucuggcuugugccucugaucuucgaucaccucagguuccugcugcucaacagaugcuaaauuuuccugagaaaaacaaggaaaaaccaguugauuugcagaacuuuggucuccguacugacauuuacuccaagaaaacauuagcaaagaguaaaggugcuagugcuggaagaggauggacugaacaggagacccuucuacuccuggaggcccuggagauguacaaggaugauuggaacaaagugucggaacauguuggaagucguacucaggaugaaugcauccuccacuuuuugagacuucccauugaggacccauaccuugagaauucagaugcuucccuugggccuuuggccuaccagccuguccccuucagucagucaggaaauccaguuaugaguacuguugcuuuuuuggcaucugugguggacccucgcguggcaucugcugcagcaaaagcggcuuuggaggaguuuucucggguccgggaggagguaccacuggaauugguugaagcucaugucaagaaaguacaagaagcagcacgagccucugggaaaguggaucccaccuacggucuggagagcagcugcauugcaggcacagggcccgaugagccagagaagcuugaaggagcugaagaggaaaaaauggaagccgacccugauggucagcagccugaaaaggcagaaaauaaaguggaaaaugaaacggaugaaggugauaaagcacaagauggagaaaaugaaaaaaauagugaaaaggaacaggauagugaagugagugaggauaccaaaucagaagaaaaggagacugaagagaacaaagaacucaguaguacauguaaagaaagagaaagugauacugggaagaagaaaguagaacaugaaauuuccgaaggaaauguugccacagccgcagcagcugcucuugccucagcggcuaccaaagccaagcaccuggcugcaguggaagaaagaaagaucaagucccugguagcucucuugguugagacacaaaugaagaaacuagagaucaaacuucgacauuuugaagggcuggaaacuaucauggacagagagaaagaagcucuagaacaacagaggcagcaguugcuuacugaacgccaaaacuuccacauggaacagcugaaguaugcugaauuacgagcacgacagcaaauggaacagcagcagcauggccagaacccucaacaggcacaccagcacucaggaggaccuggccuggccccacuuggagcagcagggcacccuggcaugaugccucaucaacagcccccucccuacccucugaugcaccaccagaugccaccaccucauccaccccagccaggucagauaccaggcccagguuccaugaugcccgggcagcacaugccaggccgcaugauucccacuguugcagccaacauccaccccucugggaguggcccuaccccuccuggcaugccaccaaugccaggaaacaucuuaggaccccggguaccccugacagcaccuaacggcauguaucccccuccaccacagcagcagccaccgccaccaccaccugcagauggggucccuccgccuccugcuccuggcccgccagccucagcugcuccuuagccuggaagaugcagggaaccuccacgcccaccaccaugagcuggaguggggaugacaagacuuguguuccucaacuuucuuggguuucuuucaggauuuuucuucucacagcuccaagcacgugucccgugccuccccacuccucuuaccaccccucucucugacacuuuuuguguuggguccucagccaacacucaaggggaaaccuguagugacagugugcccuggucauccuuaaaauaaccugcaucuccccuguccuggugugggaguaagcugacaguuucucugcagguccugucaacuuuagcaugcuaugucuuuaccauuuucucucuuccaguuuuuugcuuugucuuaugcuucuauggauaaugcuauauaaucauuaucuuuuuaucuuucuguuauuauuguuuuaaaggagagcauccuaaguuaauaggaaccaaaaaauaaugaugggcagaagggggggaauagccacaggggacaaaccuuaaggcauuauaagugaccuuauuucugcuuuucugagcuaagaauggugcugaugguaaaguuugagacuuuugccacacacaaauuugugaaaauuaaacgagauguggaaggagaaccucagugauuuuauucccuagugaggccucugagggccuccacacugccuggcagaacauaccacugaacuaguaugugcuagaggagggcacaaacauccgcuccuucccuaggccugcuggcucugguuuucuaugcagaugauucauuggauugggggugaguguuuuguuuuucugggggcagugugagcuuugaggguuggaauauugggaggcauuccuuaguuuccucaacuagccuggaaaguuaggagucuaggguaauuaccccccaaugagucuagccuacuauucacugcuuugugugcauuuuuuucucccucuuuaaaaaacccuuuaaaagaaaaaaaaaaguagauagugcuaaauauuuagcucaugaaacuugguuaggauggcuggggguacaaguccccaaacuaccucuuguuacaguagccagggaguggaauuucgucaaccgguacuuuuaagguuaggaugggacgggaaaagugaagcaggauauuagcuccuuauaccuucucccuuccauuucugagaucucacauuccaucuaucacaggguuuucaaagagaugcugaggguaacaaggaacucacuuggcagucagagcaucaugcuuugagguuuggggugcucaggcugggaggguagaaugccauuccagaggacaagccacaaaaaugccuuaauuugagcucguauuuaccccugcugauaagugacuugagaguucccgguuuuuuccucuuguccuucccucccuucuguccuuccauguguggggaaaggguguuuuugguagagcuugguuuccaaagcgccuggcuuucucacuucacauucucaaguggcaguuucauuauuuagaaugcaagguggacaucuuuuggauaucuuuuucuauauauuucuaaagcuuuacauaugagaggguauagggagguguuuauaaaacacuugagaacuuuuuuccuuaauaucagaaagcaaaaaaauaaaaccacaauugagauuugccuuucaaacccucagguuugccucuaaccaggugucccuggucaccaucagaguacuggaauacgggaaccgaggaggaccuugguccuuuuguuuuuguucuggacucuugggaguggaaaugggaugaguuuauccacuggagcuuaagucccaugcauuugcuccagaaag

SEQ ID NO: 5 - RNA, 4022 nt, Homo sapiens
ggaauuccccgagccggagaagauggcggugcggaagaaggacggcggccccaacgugaaguacuacgaggccgcggacaccgugacccaguucgacaacgugcggcuguggcucggcaagaacuacaagaaguauauacaagcugaaccacccaccaacaagucccugucuagccugguuguacaguugcuacaauuucaggaagaaguuuuuggcaaacaugucagcaaugcaccgcucacuaaacugccgaucaaauguuuccuagauuucaaagcgggaggcuccuugugccacauucuugcagcugccuacaaauucaagagugaccagggauggcggcguuacgauuuccagaauccaucacgcauggaccgcaauguggaaauguuuaugaccauugagaaguccuuggugcagaauaauugccugucucgaccuaacauuuuucugugcccagaaauugagcccaaacuacuagggaaauuaaaggacauuaucaagagacaccagggaacagucacugaggauaagaacaaugccucccauguuguguauccugucccggggaaucuagaagaagaggaauggguacgaccagucaugaagagggauaagcagguucuucugcacuggggcuacuauccugacaguuacgacacguggaucccagcgagugaaauugaggcaucuguggaagaugcuccaacuccugagaaaccuaggaagguucaugcaaaguggauccuggacaccgacaccuucaaugaauggaugaaugaggaagacuaugaaguaaaugaugacaaaaacccugucucccgccgaaagaagauuucagccaagacacugacagaugaggugaacagcccagauucagaucgacgggacaagaaggggggaaacuauaagaagaggaagcgcucccccucuccuucaccaaccccagaagucaaagaagaaaaaugcaagaaaggucccucaacaccuuacacuaagucaaagcguggccacagagaagaggagcaagaagaccugacuaaggacauggacgagcccucaccaguccccaauguagaagaggugacacuucccaaaacagucaacacaaagaaagacucagagucggccccagucaaaggcggcaccaugaccgaccuggaugaacaggaagaugaaagcauggagacgacgggcaaggaugaggaugagaacaguacggggaacaagggagagcagaccaagaauccagaccugcaugaggacaaugugacugaacagacccaccacaucaucauucccagcuacgcugccugguuugacuacaauaguguucaugccauugagcggagggcucuccccgaguucuucaacggcaagaacaaguccaagacuccagagaucuaccuggccuaucgaaacuuuaugauugacacuuaccgacugaacccccaagaguaucuuaccucuaccgccugccgccgaaaccuagcgggugaugucugugccaucucgaggguccaugccuuccuagaacaguggggucuuauuaacuaccagguggaugcugagagucgaccaaccccaauggggccuccgccuaccucucacuuccaugucuuggcugacacaccaucagggcuggugccucugcagcccaagacaccucagcagaccucugcuucccaacaaaugcucaacuuuccugacaaaggcaaagagaaaccaacagacaugcaaaacuuugggcugcgcacagacauguacacaaaaaagaaugcucccuccaagagcaaggcugcagccagugccacucgugaguggacagaacaggaaacccugcuucuccuggaggcacuggaaauguacaaagaugacuggaacaaaguguccgagcaugugggaagccgcacacaggacgagugcaucuugcauuuucuucgucuucccauugaagacccauaccuggaggacucagaggccucccuaggcccccuggccuaccaacccauccccuucagucagucgggcaacccuguuaugagcacuguugccuuccuggccucugucgucgauccccgagucgccucugcugcugcaaagucagcccuagaggaguucuccaaaaugaaggaagagguacccacggccuugguggaggcccauguucgaaaaguggaagaagcagccaaaguaacaggcaaggcggacccugccuucggucuggaaagcaguggcauugcuggaaccaccucugaugagccugagcggauugaggagagcgggaaugacgaggcucggguggaaggccaggccacagaugagaagaaggagcccaaggaaccccgagaaggagggggugcuauagaggaggaagcaaaagagaaaaccagcgaggcucccaagaaggaugaggagaaagggaaagaaggcgacagugagaaggaguccgagaagagugauggagacccaauagucgauccugagaaggagaaggagccaaaggaagggcaggaggaagugcugaaggaagugguggagucugagggggaaaggaagacaaagguggagcgggacauuggcgagggcaaccucuccaccgcugcugccgccgcccuggccgccgccgcagugaaagcuaagcacuuggcugcuguugaggaaaggaagaucaaaucuuugguggcccugcugguggagacccagaugaaaaaguuggagaucaaacuucggcacuuugaggagcuggagacuaucauggaccgggagcgagaagcacuggaguaucagaggcagcagcuccuggccgacagacaagccuuccacauggagcagcugaaguauccggagaugagggcucggcagcagcacuuccaacagaugcaccaacagcagcagcagccaccaccagcccugcccccaggcucccagccuauccccccaacaggggcugcugggccacccgcaguccauggcuuggcuguggcuccagccucuguagucccugcuccugcuggcaguggggccccuccaggaaguuugggcccuucugaacagauugggcaggcagggucaacucgagggccacagcagcagcaaccagcuggagccccccagccuggggcagucccaccagggguuccccccccuggaccccauggccccucaccguuccccaaccaacaaacuccucccucaaugaugccaggggcagugccaggcagcgggcacccaggcguggcggguaaugcuccuuuggguuugccuuuuggcaugccgccuccuccuccuccuccugcuccauccaucaucccauuugguagucuagcugacuccaucaguauuaaccuccccgcuccuccuaaccugaugggaucaccaccaucucccguucgccccgggacucuccccccaccuaaccugccuguguccauggcgaacccucuacauccuaaccugccggcgaccaccaccaugccaucuuccuugccucucgggccggggcucggauccgccgcagcccaaagcccugccauuguggcagcuguucagggcaaccuccugcccagugccagcccacugccagacccaggcaccccccugccuccagaccccacagccccgagcccaggcacggucaccccugugccaccuccacagugaggagccagccagacaucucucccccucacccccuguggacaucacgguuccaggaacagcccuucccccaccacugggacccuccccagccuggagaguucaucacuacguaaggaaagcuccuuccgccccuccaaagcccucaccaugccuaacagaggcaugcauuuuauaucaguuauucaaggacuucuguuuaaaagauguuuauaaugucugggagagaggauaggaugggaaugcugcccuaaaggaagggcuggugaagguguuauacaagguucuauuaaccacuucuaaggguacaccucccuccaaacuacugcauuuucuauggauuaaaaaaaaaaaggaauuc

SEQ ID NO: 6 - RNA, 6418 nt, Homo sapiens
ccccagcccaccgccacccccacccucaaccaacugcucacgucgcccagcucggcccggggcuaccagggcuaccccgggggcgacuacaguggcgggccccaggacgggggcgccggcaagggcccggcggacauggccucucaguguuggggggcugcggcggcggcagcugcggcggcggccgccucgggaggggcccaacaaaggagccaccacgcgcccaugagccccgggagcagcggcggcggggggcagccgcucgcccggaccccucagccauccaguccaauggaucagaugggcaagaugagaccucagccauauggcgggacuaacccauacucgcagcaacagggaccuccgucaggaccgcagcaaggacauggguacccagggcagccauacgggucccagaccccgcagcgguacccgaugaccaugcagggccgggcgcagagugccaugggcggccucucuuauacacagcagauuccuccuuauggacaacaaggccccagcggguauggucaacagggccagacuccauauuacaaccagcaaaguccucacccucagcagcagcagccacccuacucccagcaaccaccgucccagaccccucaugcccaaccuucguaucagcagcagccacagucucaaccaccacagcuccaguccucucagccuccauacucccagcagccaucccagccuccacaucagcaguccccggcuccauaccccucccagcagucgacgacacagcagcacccccagagccagccccccuacucacagccacaggcucagucuccuuaccagcagcagcaaccucagcagccagcacccucgacgcucucccagcaggcugcguauccucagccccagucucagcagucccagcaaacugccuauucccagcagcgcuucccuccaccgcaggagcuaucucaagauucauuugggucucaggcauccucagcccccucaaugaccuccaguaagggagggcaagaagauaugaaccugagccuucagucaagacccuccagcuugccugaucuaucugguucaauagaugaccuccccauggggacagaaggagcucugaguccuggagugagcacaucagggauuuccagcagccaaggagagcagaguaauccagcucagucuccuuucucuccucauaccuccccucaccugccuggcauccgaggcccuuccccguccccuguuggcucucccgccaguguugcucagucucgcucaggaccacucucgccugcugcagugccaggcaaccagaugccaccucggccacccaguggccagucggacagcaucaugcauccuuccaugaaccaaucaagcauugcccaagaucgagguuauaugcagaggaacccccagaugccccaguacaguuccccccagcccggcucagccuuaucuccgcgucagccuuccggaggacagauacacacaggcaugggcuccuaccagcagaacuccauggggagcuaugguccccaggggggucaguauggcccacaagguggcuaccccaggcagccaaacuauaaugccuugcccaaugccaacuaccccagugcaggcauggcuggaggcauaaaccccaugggugccggaggucaaaugcauggacagccuggcaucccaccuuauggcacacucccuccagggaggaugagucacgccuccaugggcaaccggccuuauggcccuaacauggccaauaugccaccucagguugggucagggaugugucccccaccagggggcaugaaccggaaaacccaagaaacugcugucgccaugcauguugcugccaacucuauccaaaacaggccgccaggcuaccccaauaugaaucaagggggcaugaugggaacuggaccuccuuauggacaagggauuaauaguauggcuggcaugaucaacccucagggacccccauauuccauggguggaaccauggccaacaauucugcagggauggcagccagcccagagaugaugggccuuggggauguaaaguuaacuccagccaccaaaaugaacaacaaggcagaugggacacccaagacagaauccaaauccaagaaauccaguucuucuacuacaaccaaugagaagaucaccaaguuguaugagcuggguggugagccugagaggaagauguggguggaccguuaucuggccuucacugaggagaaggccaugggcaugacaaaucugccugcuguggguaggaaaccucuggaccucuaucgccucuaugugucugugaaggagauugguggauugacucaggucaacaagaacaaaaaauggcgggaacuugcaaccaaccucaaugugggcacaucaagcagugcugccagcuccuugaaaaagcaguauauccagugucucuaugccuuugaaugcaagauugaacggggagaagacccucccccagacaucuuugcagcugcugauuccaagaagucccagcccaagauccagccucccucuccugcgggaucaggaucuaugcaggggccccagacuccccagucaaccagcaguuccauggcagaaggaggagacuuaaagccaccaacuccagcauccacaccacacagucagauccccccauugccaggcaugagcaggagcaauucaguugggauccaggaugccuuuaaugauggaagugacuccacauuccagaagcggaauuccaugacuccaaacccuggguaucagcccaguaugaauaccucugacaugauggggcgcauguccuaugagccaaauaaggauccuuauggcagcaugaggaaagcuccagggagugaucccuucauguccucagggcagggccccaacggcgggaugggugaccccuacagucgugcugccggcccugggcuaggaaauguggcgaugggaccacgacagcacuaucccuauggagguccuuaugacagagugaggacggagccuggaauagggccugagggaaacaugagcacuggggccccacagccgaaucucaugccuuccaacccagacucggggauguauucuccuagccgcuaccccccgcagcagcagcagcagcagcagcaacgacaugauuccuauggcaaucaguucuccacccaaggcaccccuucuggcagccccuuccccagccagcagacuacaauguaucaacagcaacagcagaauuacaagcggccaauggauggcacauauggcccuccugccaagcggcacgaaggggagauguacagcgugccauacagcacugggcaggggcagccucagcagcagcaguugcccccagcccagccccagccugccagccagcaacaagcugcccagccuuccccucagcaagauguauacaaccaguauggcaaugccuauccugccacugccacagcugcuacugagcgccgaccagcaggcggcccccagaaccaauuuccauuccaguuuggccgagaccgugucucugcacccccuggcaccaaugcccagcaaaacaugccaccacaaaugaugggcggccccauacaggcaucagcugagguugcucagcaaggcaccauguggcaggggcguaaugacaugaccuauaauuaugccaacaggcagagcacgggcucugccccccagggccccgccuaucauggcgugaaccgaacagaugaaaugcugcacacagaucagagggccaaccacgaaggcucguggccuucccauggcacacgccagcccccauauggucccucugccccugugccccccaugacaaggcccccuccaucuaacuaccagcccccaccaagcaugcagaaucacauuccucagguauccagcccugcuccccugccccggccaauggagaaccgcaccucuccuagcaagucuccauuccugcacucugggaugaaaaugcagaaggcaggucccccaguaccugccucgcacauagcaccugccccugugcagccccccaugauucggcgggauaucaccuucccaccuggcucuguugaagccacacagccuguguugaagcagaggaggcggcucacaaugaaagacauuggaaccccggaggcauggcggguaaugaugucccucaagucuggucuccuggcagagagcacaugggcauuagauaccaucaacauccugcuguaugaugacaacagcaucaugaccuucaaccucagucagcucccaggguugcuagagcuccuuguagaauauuuccgacgaugccugauugagaucuuuggcauuuuaaaggaguaugaggugggugacccaggacagagaacgcuacuggauccugggagguucagcaaggugucuaguccagcucccauggaggguggggaagaagaagaagaacuucuagguccuaaacuagaagaggaagaagaagaggaaguaguugaaaaugaugaggagauagccuuuucaggcaaggacaagccagcuucagagaauagugaggagaagcugaucaguaaguuugacaagcuuccaguaaagaucguacagaagaaugauccauuugugguggacugcucagauaagcuugggcgugugcaggaguuugacaguggccugcugcacuggcggauuggugggggggacaccacugagcauauccagacccacuucgagagcaagacagagcugcugccuucccggccucacgcacccugcccaccagccccucggaagcaugugacaacagcagaggguacaccagggacaacagaccaggaggggcccccaccugauggaccuccagaaaaacggaucacagccacuauggaugacauguugucuacucggucuagcaccuugaccgaggauggagcuaagaguucagaggccaucaaggagagcagcaaguuuccauuuggcauuagcccagcacagagccaccggaacaucaagauccuagaggacgaaccccacaguaaggaugagaccccacuguguacccuucuggacuggcaggauucucuugccaagcgcugcgucuguguguccaauaccauucgaagccugucauuugugccaggcaaugacuuugagauguccaaacacccagggcugcugcucauccugggcaagcugauccugcugcaccacaagcacccagaacggaagcaggcaccacuaacuuaugaaaaggaggaggaacaggaccaaggggugagcugcaacaaaguggagugguggugggacugcuuggagaugcuccgggaaaacaccuugguuacacucgccaacaucucggggcaguuggaccuaucuccauaccccgagagcauuugccugccuguccuggacggacuccuacacugggcaguuugcccuucagcugaagcccaggaccccuuuuccacccugggccccaaugccguccuuuccccgcagagacuggucuuggaaacccucagcaaacucagcauccaggacaacaauguggaccugauucuggccacaccccccuucagccgccuggagaaguuguauagcacuauggugcgcuuccucagugaccgaaagaacccggugugccgggagauggcugugguacugcuggccaaccuggcucagggggacagccuggcagcucgugccauugcagugcagaagggcaguaucggcaaccuccugggcuuccuagaggacagccuugccgccacacaguuccagcagagccaggccagccuccuccacaugcagaacccacccuuugagccaacuaguguggacaugaugcggcgggcugcccgcgcgcugcuugccuuggccaagguggacgagaaccacucagaguuuacucuguacgaaucacggcuguuggacaucucgguaucaccguugaugaacucauugguuucacaagucauuugugauguacuguuuuugauuggccagucaugacagccgugggacaccucccccccccgugugugugugcguguguggagaacuuagaaacugacuguugcccuuuauuuaugcaaaaccaccucagaauccaguuuacccugugcuguccagcuucucccuugggaaaaagucucuccuguuucucucuccuccuuccaccuccccucccuccaucaccucacgccuuucuguuccuuguccucaccuuacuccccucaggacccuaccccacccucuuugaaaagacaaagcucugccuacauagaagacuuuuuuuauuuuaaccaaaguuacuguuguuuacagugaguuuggggaaaaaaaauaaaauaaaaauggcuuucccaguccuugcaucaacgggaugccacauuucauaacuguuuuuaaugguaaaaaaaaaaaaaaa

SEQ ID NO: 7 - RNA, 5508 nt, Homo sapiens
auggcaaacucgacggggaaggcgccuccggacgagcggagaaagggacucgcuuuccuggacgagcugcggcaguuccaccacagcagagggucgccuuuuaaaaaaaucccugcggugggugggaaggagcuggaucuucacggucucuacaccagagucacuacuuuaggcggauucgcgaagguuucugagaagaaucaguggggagaaauuguugaagaguucaacuuucccagaaguuguucuaacgcugccuuugcuuuaaaacaguauuacuugcguuaccuagaaaaguacgagaaaguucaucauuuuggggaggaugaugaugagguaccaccgggcaauccaaagccacagcuuccuauuggugcaauuccaucuuccuacaauuaccagcaacacagugugucggauuaucugcgucaaaguuaugggcuguccauggacuuuaauucgccaaaugauuauaauaaauuggugcuuucacuguuaucuggacucccaaaugaaguggacuuugcuauuaacguaugcacucuccuaucaaaugaaagcaagcacgucaugcaacuugaaaaagauccuaaaaucaucacuuuacuacuugcuaaugccgggguguuugacgacacuuuaggauccuuuuccacuguauuuggagaagaauggaaagagaagacugauagagacuucguuaaguuuuggaaagacaucguugaugauaaugaaguucgugaccucauuucugacagaaacaagucucaugaagguacaucaggagaauggauuugggagucuuuauuucauccaccucgaaagcugggcauuaacgauauugaaggacagcggguacuucagauugcagugauuuugagaaaucuuuccuuugaggagggcaauguuaagcucuuggcagcuaaucguaccugucuucguuuccuauuacuuucugcacauagucauuuuauuucuuuaaggcaauuaggccuugacacauuaggaaauauugcagcugagcuuuuacuggacccuguugauuucaaaacuacucaucugauguuucauacuguuacaaaaugucuaaugucaagggauagauuuuuaaagaugagaggcauggaaauuuugggaaaucuuugcaaagcagaagauaaugguguuuuaauuugugaauauguggaucaggauuccuacagagagaucauuugucaucucacuuuaccugaugugcugcuuguaaucucaacacucgaggugcuauacaugcucacggaaaugggagauguugcuugcacaaaaauugcaaaaguagaaaagagcauagacauguuagugugucugguuucuauggauauucagauguuuggcccugaugcacuagcugcgguaaaacucauugaacacccaaguuccagucaucaaauguuaucugaaauuaggccacaagcuauagagcaaguccaaacccagacucauguagcaucugccccagcuuccagagcaguuguagcgcagcauguugcuccaccuccaggaauaguggaaauagauagugagaaguuugcuugucaguggcuaaaugcucauuuugaaguaaauccagauuguucuguuucucgagcagaaauguauucugaauaccucucgacuugcaguaaauuagcucgugguggaauccuaacaucaacuggauuuuauaaaugucuuagaacggucuuuccaaaucauacagugaagagaguggaggauuccaguagcaaugggcaggcacauauucaugugguaggaguaaaacggagggcuauaccacuucccauucagauguacuaucagcagcaaccaguuucuacuucuguuguucguguugauucuguuccugauguaucuccugcuccuucaccugcaggaaucccucauggaucacaaaccauaggaaaccauuuucagaggacuccuguugccaaccaaucuucaaaucugacugcaacacaaaugucuuuuccuguacaagguguucauacuguggcacaaacuguuucaagaauuccacaaaauccuucaccucauacccaccagcaacaaaaugcuccagugacugucauucaaaguaaagcuccaauuccuugugaaguuguuaaggcuacaguuauccagaauuccauaccccagacaggaguuccuguuaguauugcuguuggaggaggaccuccacagaguucuguuguucagaaucauaguacagggccacaaccuguuacaguugugaauucucagacauugcuucaccauccaucuguaauuccacagcagucuccauuacacacagugguaccaggacagaucccuucaggcacuccuguuacaguaauucaacaagcugucccacagagucauacguuuggcagaguacagaacauaccagcauguacuucuacaguuucacagggucaacaguuaaucaccacaucaccccaaccugugcaaacuucaucucaacagacaucagcugguagccagucacaagauacuguuaucauagcacccccacaguauguaacaacuucugcauccaauauugucucagcaacuucaguacagaauuuucagguagcuacaggacaaaugguuacuauugcuggugucccaaguccacaagccucaaggguaggguuucagaacauugcaccaaaaccucucccuucucagcaaguuucaucuacagugguacagcagccuauucaacaaccacagcagccaacccaacaaagcguagugauuguaagccagccagcucaacaaggucaaacuuaugcaccagccauucaccaaauuguucuugcuaauccagcagcucuuccagcuggucagacaguucagcuaacuggacaaccuaacauaacuccaucuucuucaccaucaccugucccagcuacuaauaaccaagucccuacugccaugucgucguccucuaccccucaaucacagggaccaccuccuacugucagucaaauguuaucugugaaaaggcagcaacagcagcaacauucaccagcacccccaccacagcagguacaaguacaaguucagcagccccaacaaguacagaugcaaguucaaccucaacagucgaaugcaggaguuggucagccugccucuggugagucgagucugauuaaacagcuucugcuuccgaaacgugguccuucaacaccaggugguaagcuuauucucccagcuccacagauuccucccccuaauaaugcaagagcuccuagcccucagguggucuaucagguggccaguaaccaagccgcagguuuuggagugcaggggcaaacuccagcucagcagcuauugguugggcagcaaaauguucaguuggucccaagugcaaugccacccucagggggaguacaaacugugcccauuucgaacuuacaaauauugccagguccacugaucucaaauagcccagcaaccauuuuccaagggacuucuggcaaccagguaaccauaacaguugugccaaauacgaguuuugcaccugcaacugugagucagggaaaugcaacucagcucauugcuccagcaggaauuaccaugagcggaacgcagacaggaguuggacuuccaguacaaacgcuuccagccacucaagcaucuccugcuggacaaucaucauguacuacugcuacucccccauucaaaggugauaaaauaauuugccaaaaggaggaggaagcaaaggaagcaacagguuuacauguucaugaacguaaaauugaagucauggagaacccguccugccgacgaggagccacaaacaccagcaauggggauacaaaggaaaaugaaaugcaugugggaagucuuuuaaaugggagaaaguacagugacucaagucuaccuccuucaaacucagggaaaauucaaagugagacuaaucagugcucacuaaucaguaaugggccaucauuggaauuaggugagaauggagcaucugggaaacagaacucagaacaaauagacaugcaagauaucaaaagugauuugagaaaaccgcuaguuaauggaaucugugauuuugauaaaggagaugguucucauuuaagcaaaaacauuccaaaucauaaaacuuccaaucauguaggaaauggugagauaucuccaauggaaccacaagggacuuuagauaucacucagcaagauacugccaaaggugaucaacuagaaagaauuucuaauggaccuguauuaacuuugggugguucaucugugagcaguauacaggaggcuucaaaugcggcaacacagcaauuuagugguacugauuugcuuaauggaccucuagcuucaaguuugaauucagaugugccucagcaacgcccaaguguaguugucucaccacauucuacaaccucuguuauacagggacaucaaaucauagcaguucccgacucaggaucaaaaguaucccauucuccugcccuaucaucugacguucggucuacaaauggcacagcagaaugcaaaacuguaaagaggccagcagaggauacugauagggaaacagucgcaggaauuccaaauaaaguaggaguuagaauuguuacaaucagugaccccaacaaugcuggcugcagcgcaacaaugguugcugugccagcaggagcagauccaagcacuguagcuaaaguagcaauagaaagugcuguucagcaaaagcaacagcauccaccaacauauguacagaauguggucccgcagaacacuccuaugccaccuucaccagcuguacaagugcagggccagccuaacaguucucagccuucuccauucaguggauccagucagccuggagauccaaugagaaaaccuggacagaacuucaugugucuguggcagucuuguaaaaagugguuucagacacccucacagguuuucuaccaugcagcaacugaacauggaggaaaagauguauauccagggcagugucuuugggaagguugugagccuuuucagcgacagcgguuuucuuuuauuacccacuugcaggauaagcacuguucaaaggaugcccuacuugcaggauuaaaacaagaugaaccaggacaagcaggaagucagaagucuucuaccaagcagccaacuguagggggcacaagcucaacuccuagagcacaaaaggccauugugaaucaucccagugcugcacuuauggcucugaggagaggaucaagaaaccuugucuuucgagauuuuacagaugaaaaagagggaccaauaacuaaacacauccgacuaacagcugccuuaauauuaaaaaauauugguaaauauucagaauguggucgcagauuguuaaagagacaugaaaauaacuuaucagugcuagccauuaguaacauggaagcuuccuccacccuugccaaaugccuuuaugaacuuaauuuuacaguucagaguaaggaacaagaaaaagacucagaaaugcugcaguga

SEQ ID NO: 8 - RNA, 5070 nt, Homo sapiens
aggagcaauagcagcagccguggcgccacggggcgaggcgcggcggucggugaccggccggggcugcaggcggcggacggcuggaaguuggauuccauggguuccaagagaagaagagcuaccuccccuuccagcagugucagcggggacuuugaugaugggcaccauucugugucaacaccaggcccaagcaggaaaaggaggagacuuuccaaucuuccaacuguagauccuauugccgugugccaugaacucuauaauaccauccgagacuauaaggaugaacagggcagacuucucugugagcucuucauuagggcaccaaagcgaagaaaucaaccagacuauuaugaagugguuucucagcccauugacuugaugaaaauccaacagaaacuaaaaauggaagaguaugaugauguuaauuugcugacugcugacuuccagcuucuuuuuaacaaugcaaaguccuauuauaagccagauucuccugaauauaaagccgcuugcaaacucugggauuuguaccuucgaacaagaaaugaguuuguucagaaaggagaagcagaugacgaagaugaugaugaagaugggcaagacaaucagggcacagugacugaaggaucuucuccagcuuacuugaaggagauccuggagcagcuucuugaagccauaguuguagcuacaaauccaucaggacgucucauuagcgaacuuuuucagaaacugccuucuaaagugcaauauccagauuauuaugcaauaauuaaggagccuauagaucucaagaccauugcccagaggauacagaauggaagcuacaaaaguauucaugcaauggccaaagauauagaucuccucgcaaaaaaugccaaaacuuauaaugagccuggcucucaaguauucaaggaugcaaauucaauuaaaaaaauauuyuauaugaaaaaggcugaaauugaacaucaugaaauggcuaagucaagucuucgaaugaggacuccauccaaccuugcugcagccagacugacagguccuucacacaguaaaggcagccuuggugaagagagaaaucccacuagcaaguauuaccguaauaaaagagcaguacaaggaggucguuuaucagcaauuacaauggcacuucaauauggcucagaaagugaagaagaugcugcuuuagcugcugcacgcuaugaagagggagagucagaagcagaaagcaucacuuccuuuauggauguuucaaauccuuuuuaucagcuuuaugacacaguuaggaguugucggaauaaccaagggcagcuaauagcugaaccuuuuuaccauuugccuucaaagaaaaaauacccugauuauuaccagcaaauuaaaaugcccauaucacuacaacagauccgaacaaaacugaagaaucaagaauaugaaacuuuagaucauuuggagugugaucugaauuuaauguuugaaaaugccaaacgcuauaaugugcccaauucagccaucuacaagcgaguucuaaaauugcagcaaguuaugcaggcaaagaagaaagagcuugccaggagagacgauaucgaggacggagacagcaugaucucuucagccaccucugauacugguagugccaaaagaaaaaguaaaaagaacauaagaaagcagcgaaugaaaaucuuauucaauguuguucuugaagcucgagagccagguucaggcagaagacuuugugaccuauuuaugguuaaaccauccaaaagggacuauccugauuauuauaaaaucaucuuggagccaauggacuugaaaauaauugagcauaacauccgcaaugacaaauaugcuggugaagagggaaugauagaagacaugaagcugauguuccggaaugccaggcacuauaaugaggagggcucccagguuuauaaugaugcacauauccuggagaaguuacucaaggagaaaaggaaagagcugggcccacugccugaugaugaugacauggcuucucccaaacucaagcugaguaggaagaguggcauuucuccuaaaaaaucaaaauacaugacuccaaugcagcagaaacuaaaugaggucuaugaagcuguaaagaacuauacugauaagaggggucgccgccucagugccauauuucugaggcuucccucuagaucugaguugccugacuacuaucugacuauuaaaaagcccauggacauggaaaaaauucgaagucacaugauggccaacaaguaccaagauauugacucuaugguugaggacuuugucaugauguuuaauaaugccuguaccuacaaugaaccggagucuuugaucuacaaagaugcucuuguucuacacaaaguccugcuugaaacacgcagagaccuggagggagaugaggacucucaugucccaaaugugacuuugcugauucaagagcuuauccacaaucuuuuugugucagucaugagucaucaggaugaugagggaagaugcuacagcgauucuuuagcagaaauuccugcuguggaucccaacuuuccuaacaaaccaccccuuacauuugacauaauuaggaagaauguugaaaauaaucgcuaccgucggcuugauuuauuucaagagcauauguuugaaguauuggaacgagcaagaaggaugaaucggacagauucagaaauauaugaagaugcaguagaacuucagcaguuuuuuauuaaaauucgugaugaacucugcaaaaauggagagauucuucuuucaccggcacucagcuauaccacaaaacauuugcauaaugauguggagaaagagagaaaggaaaaauugccaaaagaaauagaggaagauaaacuaaaacgagaagaagaaaaaagagaagcugaaaagagugaagauuccucuggugcugcaggccucucaggcuuacaucgcacauacagccaggacuguagcuuuaaaaacagcauguaccauguuggagauuacgucuauguggaaccugcagaggccaaccuacaaccacauaucgucuguauugaaagacugugggaggauucagcugaaaaagaaguuuuuaagagugacuauuacaacaaaguuccaguuaguaaaauucuaggcaaguguguggucauguuugucaaggaauacuuuaaguuaugcccagaaaacuuccgagaugaggauguuuuugucugugaaucacgguauucugccaaaaccaaaucuuuuaagaaaauuaaacuguggaccaugcccaucagcucagucagguuugucccucgggaugugccucugccugugguucgcguggccucuguauuugcaaaugcagauaaaggugaugaugagaagaauacagacaacucagaggacagucgagcugaagacaauuuuaacuuggaaaaggaaaaagaagaugucccuguggaaauguccaauggugaaccaguuugccacuacuuugagcagcuccauuacaaugacauguggcugaagguuggcgacugugucuucaucaagucccauggccuggugcguccucgugugggcagaauugaaaaaguauggguucgagauggagcugcauauuuuuauggccccaucuucauucacccagaagaaacagagcaugagcccacaaaaauguucuacaaaaaagaaguauuucugaguaaucuggaagaaaccugccccaugacauguauucucggaaagugugcuguguugucauucaaggacuuccucuccugcaagccaacugaaauaccagaaaaugacauucugcuuugugagagccgcuacaaugagagcgacaagcagaugaagaaauucaaaggauugaagagguuuucacucucugcuaaagugguagaugaugaaauuuacuacuucagaaaaccaauuguuccucagaaggagccaucaccuuugcugggaaagaagauucaguugcuagaagcuaaauuugccgaguuagaagguggagaugaugauauugaagagaugggagaagaagauagugagucuaccccaaagucugccaaaggcagugcaaagaaggaaggcuccaaacggaaaaucaacaugaguggcuacauccuguucagcagugagaugagggcugugauuaaggcccaacacccagacuacucuuucggggagcucagccgccugguggggacagaauggagaaaucuugagacagccaagaaagcagaauaugaaggcaugauggguggcuauccgccaggccuuccaccuuugcagggcccaguugauggccuuguuagcaugggcagcaugcagccacuucacccuggggggccuccaccccaccaucuuccgccaggugugccuggccucccgggcaucccaccaccgggugugaugaaccaaggaguggccccuaugguagggacuccagcaccggguggaaguccauauggacaacaggugggaguuuuggggccuccaaggcagcaggcaccaccuccauaucccggcccacauccagcuggacccccugucauacagcagccaacaacacccauguuuguagcucccccgccaaagacccagcggcuucuucacucagaggccuaccugaaauacauugaaggacucagugcggaguccaacagcauuagcaagugggaucagacacuggcagcucgaagacgcgacguccauuugucgaaagaacaggagagccgccuacccucucacuggcugaaaagcaaaggggcccacaccaccauggcagaugcccucuggcgccuucgagauuugaugcuccgggacacccucaacauucgccaagcauacaaccuagaaaauguuuaaucacaucauuacguuucuuuuauauagaagcauaaagaguuguggaucaguagccauuuuaguuacuggggguggggggaaggaacaaaggaggauaauuuuuauugcauuuuacuguacaucacaaggccauuuuuauauacggacacuuuuaauaagcuauuucaauuuguuuguuauauuaaguugacuuuaucaaauacacaaagauuuuuuugcauaaaa

SEQ ID NO: 9 - RNA, 2576 nt, Homo sapiens
gaauuccgggcgacgcgcgggaacaacgcgagucggcgcgcgggacgaagaauaaucaugggccagacugggaagaaaucugagaagggaccaguuuguuggcggaagcguguaaaaucagaguacaugcgacugagacagcucaagagguucagacgagcugaugaaguaaagaguauguuuaguuccaaucgucagaaaauuuuggaaagaacggaaaucuuaaaccaagaauggaaacagcgaaggauacagccugugcacauccugacuucugugagcucauugcgcgggacuagggaguguucggugaccagugacuuggauuuuccaacacaagucaucccauuaaagacucugaaugcaguugcuucaguacccauaauguauucuuggucuccccuacagcagaauuuuaugguggaagaugaaacuguuuuacauaacauuccuuauaugggagaugaaguuuuagaucaggaugguacuuucauugaagaacuaauaaaaaauuaugaugggaaaguacacggggauagagaauguggguuuauaaaugaugaaauuuuuguggaguuggugaaugcccuuggucaauauaaugaugaugacgaugaugaugauggagacgauccugaagaaagagaagaaaagcagaaagaucuggaggaucaccgagaugauaaagaaagccgcccaccucggaaauuuccuucugauaaaauuuuggaggccauuuccucaauguuuccagauaagggcacagcagaagaacuaaaggaaaaauauaaagaacucaccgaacagcagcucccaggcgcacuuccuccugaauguacccccaacauagauggaccaaaugcuaaaucuguucagagagagcaaagcuuacacuccuuucauacgcuuuucuguaggcgauguuuuaaauaugacugcuuccuacauccuuuucaugcaacacccaacacuuauaagcggaagaacacagaaacagcucuagacaacaaaccuuguggaccacaguguuaccagcauuuggagggagcaaaggaguuugcugcugcucucaccgcugagcggauaaagaccccaccaaaacguccaggaggccgcagaagaggacggcuucccaauaacaguagcaggcccagcacccccaccauuaaugugcuggaaucaaaggauacagacagugauagggaagcagggacugaaacggggggagagaacaaugauaaagaagaagaagagaagaaagaugaaacuucgagcuccucugaagcaaauucucggugucaaacaccaauaaagaugaagccaaauauugaaccuccugagaauguggaguggaguggugcugaagccucaauguuuagaguccucauuggcacuuacuaugacaauuucugugccauugcuagguuaauugggaccaaaacauguagacagguguaugaguuuagagucaaagaaucuagcaucauagcuccagcucccgcugaggauguggauacuccuccaaggaaaaagaagaggaaacaccgguugugggcugcacacugcagaaagauacagcugaaaaaggacggcuccucuaaccauguuuacaacuaucaacccugugaucauccacggcagccuugugacaguucgugcccuugugugauagcacaaaauuuuugugaaaaguuuugucaauguaguucagagugucaaaaccgcuuuccgggaugccgcugcaaagcacagugcaacaccaagcagugcccgugcuaccuggcuguccgagagugugacccugaccucugucuuacuuguggagccgcugaccauugggacaguaaaaauguguccugcaagaacugcaguauucagcggggcuccaaaaagcaucuauugcuggcaccaucugacguggcaggcugggggauuuuuaucaaagauccugugcagaaaaaugaauucaucucagaauacuguggagagauuauuucucaagaugaagcugacagaagagggaaaguguaugauaaauacaugugcagcuuucuguucaacuugaacaaugauuuugugguggaugcaacccgcaaggguaacaaaauucguuuugcaaaucauucgguaaauccaaacugcuaugcaaaaguuaugaugguuaacggugaucacaggauagguauuuuugccaagagagccauccagacuggcgaagagcuguuuguugauuacagauacagccaggcugaugcccugaaguaugucggcaucgaaagagaaauggaaaucccuugacaucugcuaccuccucccccuccucugaaacagcugccuuagcuucaggaaccucgaguacugugggcaauuuagaaaaagaacaugcaguuugaaauucugaauuugcaaaguacuguaagaauaauuuauaguaaugaguuuaaaaaucaacuuuuuauugccuucucaccagcugcaaaguguuuuguaccagugaauuuuugcaauaaugcaguaugguacauuuuucaacuuugaauaaagaauacuugaacuugaaaaaaaaaaaaaaaaaa

SEQ ID NO: 10 - RNA, 4441 nt, Homo sapiens
cucugaggagacacuuuuuuuuuccucccuccuucccuccucuccuccucccuucccuuccccucuccuccccucucuccuccuuccccccucgguccgccggagccugcuggggcgagcgguugguauugcaggcgcuugcucuccggggccgcccggcggguagcuggcggggggaggaggcaggaaccgcgauggcgccucagaagcacggcggugggggagggggcggcucggggcccagcgcgggguccgggggaggcggcuucggggguucggcggcgguggcggcggcgacggcuucgggcggcaaauccggcggcgggagcuguggaggggguggcaguuacucggccuccuccuccuccuccgcggcggcagcggcgggggcugcgguguuaccggugaagaagccgaaaauggagcacguccaggcugaccacgagcuuuuccuccaggccuuugagaagccaacacagaucuauagauuucuucgaacucggaaucucauagcaccaauauuuuugcacagaacucuuacuuacaugucucaucgaaacuccagaacaaacaucaaaaggaaaacauuuaaaguugaugauauguuaucaaaaguagagaaaaugaaaggagagcaagaaucucauagcuugucagcucauuugcagcuuacguuuacugguuucuuccacaaaaaugauaagccaucaccaaacucagaaaaugaacaaaauucuguuacccuggaaguccugcuugugaaaguuugccacaaaaaaagaaaggauguaaguuguccaauaaggcaaguucccacagguaaaaagcaggugccuuugaauccugaccucaaucaaacaaaacccggaaauuucccgucccuugcaguuuccaguaaugaauuugaaccuaguaacagccauauggugaagucuuacucguugcuauuuagagugacucguccaggaagaagagaguuuaauggaaugauuaauggagaaaccaaugaaaauauugaugucaaugaagagcuuccagccagaagaaaacgaaaucgugaggauggggaaaagacauuuguugcacaaaugacaguauuugauaaaaacaggcgcuuacagcuuuuagauggggaauaugaaguagccaugcaggaaauggaagaauguccaauaagcaagaaaagagcaacaugggagacuauucuugaugggaagaggcugccuccauucgaaacauuuucucagggaccuacguugcaguucacucuucguuggacaggagagaccaaugauaaaucuacggcuccuauugccaaaccucuugccacuagaaauucagagagucuccaucaggaaaacaagccugguucaguuaaaccuacucaaacuauugcuguuaaagaaucauugacuacagaucuacaaacaagaaaagaaaaggauacuccaaaugaaaaccgacaaaaauuaagaauauuuuaucaguuucucuauaacaacaauacaaggcaacaaacugaagcaagagaugaccugcauugcccuugguguacucugaacugccgcaaacuuuauaguuuacucaagcaucuuaaacucugccauagcagauuuaucuucaacuauguuuaucauccaaaaggugcuaggauagauguuucuaucaaugaguguuaugauggcuccuaugcaggaaauccucaggauauucaucgccaaccuggauuugcuuuuagucgcaacggaccaguuaagagaacaccuaucacacauauucuugugugcaggccaaaacgaacaaaagcaagcaugucugaauuucuugaaucugaagauggggaaguagaacagcaaagaacauauaguaguggccacaaucgucuguauuuccauagugauaccugcuuaccucuccguccacaagaaauggaaguagauagugaagaugaaaaggauccugaauggcuaagagaaaaaaccauuacacaaauugaagaguuuucugauguuaaugaaggagagaaagaagugaugaaacucuggaaucuccaugucaugaagcauggguuuauugcugacaaucaaaugaaucaugccuguaugcuguuuguagaaaauuauggacagaaaauaauuaagaagaauuuaugucgaaacuucaugcuucaucuagucagcaugcaugacuuuaaucuuauuagcauaaugucaauagauaaagcuguuaccaagcuccgugaaaugcagcaaaaauuagaaaagggggaaucugcuuccccugcaaacgaagaaauaacugaagaacaaaaugggacagcaaauggauuuagugaaauuaacucaaaagagaaagcuuuggaaacagauagugucucagggguuucaaaacagagcaaaaaacaaaaacucugaaaagcucuaaccccauguuauggacaaacacugaaauuacauuuuagggaauucauccucuaagaauuauguuuuuguuuuuaaucauauguuccaaacaggcacuguuagaugaaguaaaugauuucaacaaggauauuuguaucaggguucuacuucacuucauuaugcagcauuacauguauaucacuuuuauugaugucauuaaaacauucuguacuuuaagcaugaaaagcaauauuucaaaguauuuuuaaacucaacaaaugucaucaaauauguugaauugaucuagaaauuauuucauauauaaaucagaauuuuuuugcauuuaugaacggcuguuuuucuacuuuguaauugugagacauuuucuuggggagggaaaauuggaaugguucccuuuuuuagaaauugaaguggucuucauaugucaacuacagaaaaggaaaaaaauagaaauugaaggauuuuuaugaaauuauauugcauuacuauuugcagucaaacuuugauccuuguuuuugaaaucauuugucaauucggaaugaaaaauuauaauguaauuuuacauuacauaaguuccuuuuacaauuaaaaaauagcacuucuucaucuuaugccuguuugagaagauauuaaauuuucacauuguugacagugaaaugcuauguugguuuauaagauuacagaccauuuguuuucauguggauaauuuuagugcauugcucacccgguauguuuuuuuuuuuuaacuugaacauuuugcuuguuuuguuuuucuuuuuuaauuagauaaucacacggaaaauuaagcuguucauaucuuuaaauuaggauugcaaaccaaggaaagaacgcauuugagauuuuaagaugucacuuauaaggggagaaguguucuuaaaaagucaaccagaaaacuguuaugccuuuuauuuguuugcaaggaugucuuuguaauguguuucaugaauagaauauccaauagagauaagcugacuugaaucauuuugagcaauuuugcccuguguuauauguguuucacgcacauauuugcaguuggauuuucuccaacagaaaguggauucacuacuggcacauuaacaagcaccaauagguuuuuauuccaacuccgagcacugugguugaguaacaucaccucaauuuuuuauuauccuuaaagauauugcauuuucauauucuuuauuuauaaaggaucaaugcugcuguaaauacagguauuuuuaauuuuaaaauuucauuccaccaccaucagaugcaguucccuauuuuguuuaaugaagggauauauaagcuuucuaauggugucuucagaaauuuauaaaauguaaauacugauuugacuggucuuuaagauguguuuaacugugaggcuauuuaacgaauaguguggaugugauuugucauccaguauuaaguucuuagucauugauuuuuguguuuaaaaaaaaauaggaaagagggaaacugcagcuuucauuacagauuccuugauugguaagcucuccaaaugaugaguucuaguaaacucugauuuuugccucuggauaguagaucucgagcguuuaucucgggcuuuaauuugcuaaagcugugcacauauguaaaaaaaaaaaaaaaaagauuauuuuaggggagauguagguguagaauuauugcuuaugucauuucuuaagcaguuaugcucuuaaugcuuaaaagaaggcuagcauuguuugcacaaaaaguuggugauucccaccccaaauaguaauaaaauuacuucuguugaguaaacuuuuuaugucaucguaaaagcuggaaaaaucccuuuguuucuauuuauaaaaaaagugcuuuucuauauguacccuugauaacagauuuugaagaaauccuguaagaugauaaagcauuugaaugguacaguagauguaaaaaaaauucaguuuaaaagaacauuuguuuuuacauuaaauguuuauuugaaaucaaaugauuuuguacauaaaguucaauaauau

SEQ ID NO: 11 - RNA, 1879 nt, Homo sapiens
gggggaagggagacauacuuaauacugcccucuuaauccaacggaccuuacaucguguagacugccgggagggcggcgggaaaagggcaagacgggaguuggggaagggaaggagccaggaagccgcgcgggagggcgcgcgcgcgcgccccuuuuucagcaguguggcggggucgcacgcacgcccgccucggcggcugggcgcgauuugcgacaguggggggggcgguggagguggcggcggcagcggcaacuuugcggcaagcucgggccgggcuugcuugacggcgguguggcggaggccccgccccaggcggcaggaaccuggagggaggcggaggaauauguccgagagggaagugucgacugcgccggcgggaacagacaugccugcggccaagaagcagaagcugagcagugacgagaacagcaauccagaccucucuggagacgagaaugaugacgcugucaguauagaaagugguacaaacacugaacgcccugauacaccuacaaacacgccaaaugcaccuggaaggaaaaguuggggaaagggaaaauggaagucaaagaaaugcaaauauucuuucaaauguguaaauagucucaaggaagaucauaaccaaccauuguuuggaguucaguuuaacuggcacaguaaagaaggagauccauuaguguuugcaacuguaggaagcaacagaguuaccuuguaugaaugucauucacaaggagaaauccgguuguugcaaucuuacguggaugcugaugcugaugaaaacuuuuacacuugugcauggaccuaugauagcaauacgagccauccucugcuggcuguagcuggaucuagaggcauaauuaggauaauaaauccuauaacaaugcaguguauaaagcacuauguuggccauggaaaugcuaucaaugagcugaaauuccauccaagagauccaaaucuucuccugucaguaaguaaagaucaugcuuuacgauuauggaauauccagacggacacucugguggcaauauuuggaggcguagaagggcacagagaugaaguucuaagugcugauuaugaucuuuugggugaaaaaauaauguccugugguauggaucauucucuuaaacuuuggaggaucaauucaaagagaaugaugaaugcaauuaaggaaucuuaugauuauaauccaaauaaaacuaacaggccauuuauuucucagaaaauccauuuuccugauuuuucuaccagagacauacauaggaauuauguugauugugugcgaugguuaggcgauuugauacuuucuaagucuugugaaaaugccauugugugcuggaaaccuggcaagauggaagaugauauagauaaaauuaaacccagugaaucuaaugugacuauucuugggcgauuugauuacagccagugugacauuugguacaugagguuuucuauggauuucuggcaaaagaugcuugcauugggcaaucaaguuggcaaacuuuauguuugggauuuagaaguagaagauccucauaaagccaaauguacaacacugacucaucauaaauguggugcugcuauucgacaaaccaguuuuagcagggauagcagcauucuuauagcuguuugugaugaugccaguauuuggcgcugggaucgacuucgauaaaauacuuuugccuaaucaaaauuagaguguguuuguugucuguguaaaauagaauuaauguaucuugcuaguaagggcacguagagcauuuagaguugucuuucagcauucaaucaggcugagcugaauguagugauguuuacauuguuuacauucuuuguacugucuuccugcucagacucuacugcuuuuaauaaaaauuuauuuuu

SEQ ID NO: 12 - RNA, 4068 nt, Homo sapiens
guuuuacuaaagugaauuuuuuuuuguuugcuucguucgucuuuggcucuuuuuuuuuccuucccaauuucggauuuauuucaaggcgaaucuggcuuugggggaagaggaagaaaagucggauuacaagaucaaccaccaccaacaacaauaaaaaccaccaggauauuuuuuugcaaauuucugacggcuuuaaauucaugaagcaauuguccccuuuugcaaucagcauuuggaucucagaaugagcaaggaaagacccaagaggaauaucauucagaagaaauacgaugacagugaugggauuccguggucagaagaacgggugguacguaaaguccuuuauuugucccugaaggaauucaagaauucccagaagaggcagcaugcggaaggcauugcugggagccugaaaacugugaaugggcuccuugguaaugaccagucuaagggauuaggaccagcaucagaacagucagagaaugaaaaggacgaugcaucccaaguguccuccacuagcaacgauguuaguucuucagauuuugaagaagggccgucgaggaaaaggcccaggcugcaagcacaaaggaaguuugcucagucucagccgaauagucccagcacaacuccaguaaagauaguggagccauugcuacccccuccagcuacucagauaucagaccucucuaaaaggaagccuaagacagaagauuuucuuaccuuucucugccuucgagguucuccugcgcugcccaacagcaugguguauuuuggaagcucucaggaugaggaggaagucgaggaggaagaugaugagacagaagacgucaaaacagccaccaacaaugcuucaucuucaugccagucgacccccaggaaaggaaaaacccacaaacauguucacaacgggcauguuuucaaugguuccagcaggucaacacgggagaaggaaccuguucaaaaacacaaaagcaaagaggccacucccgcaaaggagaagcacagcgaucaccgggcugacagccgccgggagcaggcuucagcuaaccaccccgcagcggcccccuccacggguuccucggccaaggggcuugcugccacccaucaccaccccccucugcaucggucggcucaggacuuacggaaacagguuucuaagguaaacggagucacucgaaugucaucucugggugcagguguaaccagugccaaaaagaugcgcgaggucagaccuucaccauccaaaacugugaaguacacugccacggugacgaagggggcugucacauacaccaaagccaagagagaacuggucaaggacaccaaacccaaucaccacaagcccaguuccgcugucaaccacacaaucucagggaaaacugaaaguagcaaugcaaaaacccgcaaacaggugcuaucccucgggggggcguccaaguccacugggcccgccgucaauggccucaaggucaguggcagguugaacccaaagucaugcacuaaggaggugggggggcggcagcugcgggagggccugcagcugcgggaggggcugcggaacuccaagaggagacuggaagaggcacaccaggcggagaagccgcagucgccccccaagaagaugaaaggggcggcuggccccgccgaaggcccuggcaagaaggccccggccgagagaggucugcugaacggacacgugaagaaggaagugccggagcgcagucuggagaggaaucggccgaagcgggccacggccgggaagagcacgccaggcagacaagcacauggcaaggcggacagcgccuccugugaaaaucguucuaccucgcaaccggaguccgugcacaagccgcaggacucgggcaaggccgagaagggcggcggcaaggccgggugggcggccauggacgagauccccguccucaggcccuccgccaaggaguuccacgauccgcucaucuacaucgagucgguccgcgcucagguggagaaguucgggaugugcagggugauccccccuccggacuggcggcccgagugcaagcucaacgaugagaugcgguuugucacgcagauucagcacauccacaagcugggccggcgcuggggccccaacgugcagcggcuggccugcaucaagaagcaccucaaaucucagggcaucaccauggacgagcucccgcucauagggggcugugagcucgaccuggccugcuuuuuccggcugauuaaugagaugggcggcaugcaacaagugacugaacucaaaaaauggaacaaacuaucagacaugcugcgcauccccaaaacugcccaggaacggcuggccaagcugcaggaagccuacugccaguacauacuuucguaugacucccuguccccagaggagcaccggcggcuggagaaggaggugcugauggagaaggagauccuggagaagcgcaaggggccgcuggaaggccacacagagaacgaccaccacaaguuccacccucugccccgcuuagagcccaagaaugggcucauccacggcguggcccccaggaacggcuuccgcagcaagcucaaggaggugggccaggcccaguugaagacuggccggcggcgacucuucgcucaggaaaaagaaguggucaaggaagaggaggaggacaaaggcguccucaaugacuuccacaagugcaucuauaagggaaggucuguuucucuaacaacuuuuuaucgaacagcgaggaauaucaugagcauguguuucagcaaggagccugccccagccgaaaucgagcaagaguacuggaggcuaguggaagagaaggacugccacguggcagugcacugcggcaagguggacaccaacacucacggcaguggauucccaguaggaaaaucagaacccuuuucgaggcauggauggaaccucaccguccuccccaauaacacaggguccauccugcgucaccucggugcugugccuggagugacuauucccuggcuaaauauuggcauggucuuuucuaccucaugcuggucucgagaccaaaaucaccuuccauacauugacuacuuacacacuggugcugacugcauuugguauugcauuccugcugaggaggagaacaagcuggaagaugugguccacacccugcugcaagccaauggcaccccagggcugcagaugcuggaaagcaacgucaugaucuccccggaggugcugugcaaagaggggaucaaggugcacaggaccgugcagcagaguggccaguuugucgucugcuucccgggauccuuuguguccaaagugugcuguggguacagcgugucugaaaccgugcacuuugcuaccacccaguggacaaguaugggcuuugagaccgccaaggaaaugaagcgucgccauauagcuaagccauucuccauggagaaguuacucuaccagauugcacaagcagaagcaaaaaaagaaaacggucccacucucaguaccaucucagcccuccuggaugagcucagggauacagagcuacggcagcgcaggcagcuguucgaggcuggccuccacuccuccgcacgcuauggcagccacgauggcagcagcacgguggcggacgggaagaaaaagccucgaaaguggcugcaguuggagacgucagagaggaggugucagaucugccagcaccugugcuaccuguccauggugguacaagagaacgaaaacgucguguucugucuggagugugcucugcgccacguggagaaacagaaguccugccgagggcugaaguugauguaccgcuacgaugaggaacagauuaucagucuggucaaucagaucugcggcaaagugucugguaaaaacggcagcauugagaacugucuccauaaacccacaccaaaaagagguccccgcaagagagcgacaguggacgugccccccucccgugcugucagccuccaguucauccaaaagugcuucgagcuacaucaugaagaugcccaacgcccguggucgauuuauauauauuuuuuuguaauuauuauauucuaguuuggaguacuugcuguaggauc

SEQ ID NO: 13 - protein, 1590 aa, Homo sapiens
MSTPTDPGAMPHPGPSPGPGPSPGPILGPSPGPGPSPGSVHSMMGPSPGPPSVSHPMPTMGSTDFPQEGMHQMHKPIDGIHDKGIVEDIHCGSMKGTGMRPPHPGMGPPQSPMDQHSQGYMSPHPSPLGAPEHVSSPMSGGGPTPPQMPPSQPGALIPGDPQAMSQPNRGPSPFSPVQLHQLRAQILAYKMLARGQPLPETLQLAVQGKRTLPGLQQQQQQQQQQQQQQQQQQQQQQQPQQQPPQPQTQQQQQPALVNYNRPSGPGPELSGPSTPQKLPVPAPGGRPSPAPPAAAQPPAAAVPGPSVPQPAPGQPSPVLQLQQKQSRISPIQKPQGLDPVEILQEREYRLQARIAHRIQELENLPGSLPPDLRTKATVELKALRLLNFQRQLRQEVVACMRRDTTLETALNSKAYKRSKRQTLREARMTEKLEKQQKIEQERKRRQKHQEYLNSILQHAKDFKEYHRSVAGKIQKLSKAVATWHANTEREQKKETERIEKERMRRLMAEDEEGYRKLIDQKKDRRLAYLLQQTDEYVANLTNLVWEHKQAQAAKEKKKRRRRKKKAEENAEGGESALGPDGEPIDESSQMSDLPVKVTHTETGKVLFGPEAPKASQLDAWLEMNPGYEVAPRSDSEESDSDYEEEDEEEESSRQETEEKILLDPNSEEVSEKDAKQIIETAKQDVDDEYSMQYSARGSQSYYTVAHAISERVEKQSALLINGTLKHYQLQGLEWMVSLYNNNLNGILADEMGLGKTIQTIALITYLMEHKRLNGPYLIIVPLSTLSNWTYEFDKWAPSVVKISYKGTPAMRRSLVPQLRSGKFNVLLTTYEYIIKDKHILAKIRWKYMIVDEGHRMKNHHCKLTQVLNTHYVAPRRILLTGTPLQNKLPELWALLNFLLPTIFKSCSTFEQWFNAPFAMTGERVDLNEEETILIIRRLHKVLRPFLLRRLKKEVESQLPEKVEYVIKCDMSALQKILYRHMQAKGILLTDGSEKDKKGKGGAKTLMNTIMQLRKICNHPYMFQHIEESFAEHLGYSNGVINGAELYRASGKFELLDRILPKLRATNHRVLLFCQMTSLMTIMEDYFAFRNFLYLRLDGTTKSEDRAALLKKFNEPGSQYFIFLLSTRAGGLGLNLQAADTVVIFDSDWNPHQDLQAQDRAHRIGQQNEVRVLRLCTVNSVEEKILAAAKYKLNVDQKVIQAGMFDQKSSSHERRAFLQAILEHEEENEEEDEVPDDETLNQMIARREEEFDLFMRMDMDRRREDARNPKRKPRLMEEDELPSWIIKDDAEVERLTCEEEEEKIFGRGSRQRRDVDYSDALTEKQWLRAIEDGNLEEMEEEVRLKKRKRRRNVDKDPAKEDVEKAKKRRGRPPAEKLSPNPPKLTKQMNAIIDTVINYKDRCNVEKVPSNSQLEIEGNSSGRQLSEVFIQLPSRKELPEYYELIRKPVDFKKIKERIRNHKYRSLGDLEKDVMLLCHNAQTFNLEGSQIYEDSIVLQSVFKSARQKIAKEEESEDESNEEEEEEDEEESESEAKSVKVKIKLNKKDDKGRDKGKGKKRPNRGKAKPVVSDFDSDEEQDEREQSEGSGTDDE

SEQ ID NO: 14 - protein, 1647 aa, Homo sapiens
MSTPDPPLGGTPRPGPSPGPGPSPGAMLGPSPGPSPGSAHSMMGPSPGPPSAGHPIPTQGPGGYPQDNMHQMHKPMESMHEKGMSDDPRYNQMKGMGMRSGGHAGMGPPPSPMDQHSQGYPSPLGGSEHASSPVPASGPSSGPQMSSGPGGAPLDGADPQALGQQNRGPTPFNQNQLHQLRAQIMAYKMLARGQPLPDHLQMAVQGKRPMPGMQQQMPTLPPPSVSATGPGPGPGPGPGPGPGPAPPNYSRPHGMGGPNMPPPGPSGVPPGMPGQPPGGPPKPWPEGPMANAAAPTSTPQKLIPPQPTGRPSPAPPAVPPAASPVMPPQTQSPGQPAQPAPMVPLHQKQSRITPIQKPRGLDPVEILQEREYRLQARIAHRIQELENLPGSLAGDLRTKATIELKALRLLNFQRQLRQEVVVCMRRDTALETALNAKAYKRSKRQSLREARITEKLEKQQKIEQERKRRQKHQEYLNSILQHAKDFKEYHRSVTGKIQKLTKAVATYHANTEREQKKENERIEKERMRRLMAEDEEGYRKLIDQKKDKRLAYLLQQTDEYVANLTELVPQHKAAQVAKEKKKKKKKKKAENAEGQTPAIGPDGEPLDETSQMSDLPVKVIHVESGKILTGTDAPKAGQLEAWLEMNPGYEVAPRSDSEESGSEEEEEEEEEEQPQAAQPPTLPVEEKKKIPDPDSDDVSEVDARHIIENAKQDVDDEYGVSQALARGLQSYYAVAHAVTERVDKQSALMVNGVLKQYQIKGLEWLVSLYNNNLNGILADEMGLGKTIQTIALITYLMEHKRINGPFLIIVPLSTLSNWAYEFDKWAPSVVKVSYKGSPAARRAFVPQLRSGKFNVLLTTYEYIIKDKHILAKIRWKYMIVDEGHRMKNHHCKLTQVLNTHYVAPRRLLLTGTPLQNKLPELWALLNFLLPTIFKSCSTFEQWFNAPFAMTGEKVDLNEEETILIIRRLHKVLRPFLLRRLKKEVEAQLPEKVEYVIKCDMSALQRVLYRHMQAKGVLLTDGSEKDKKGKGGTKTLMNTIMQLRKICNHPYMFQHIEESFSEHLGFTGGIVQGLDLYRASGKFELLDRILPKLRATNHKVLLFCQMTSLMTIMEDYFAYRGFKYLRLDGTTKAEDRGMLLKTFNEPGSEYFIFLLSTRAGGLGLNLQSADTVIIFDSDWNPHQDLQAQDRAHRIGQQNEVRVLRLCTVNSVEEKILAAAKYKLNVDQKVIQAGMFDQKSSSHERRAFLQAILEHEEQDESRHCSTGSGSASFAHTAPPPAGVNPDLEEPPLKEEDEVPDDETVNQMIARHEEEFDLFMRMDLDRRREEARNPKRKPRLMEEDELPSWIIKDDAEVERLTCEEEEEKMFGRGSRHRKEVDYSDSLTEKQWLKAIEEGTLEEIEEEVRQKKSSRKRKRDSDAGSSTPTTSTRSRDKDDESKKQKKRGRPPAEKLSPNPPNLTKKMKKIVDAVIKYKDSSSGRQLSEVFIQLPSRKELPEYYELIRKPVDFKKIKERIRNHKYRSLNDLEKDVMLLCQNAQTFNLEGSLIYEDSIVLQSVFTSVRQKIEKEDDSEGEESEEEEEGEEEGSESESRSVKVKIKLGRKEKAQDRLKGGRRRPSRGSRAKPVVSDDDSEEEQEEDRSGSGSEED

SEQ ID NO: 15 - protein, 385 aa, Homo sapiens
MMMMALSKTFGQKPVKFQLEDDGEFYMIGSEVGNYLRMFRGSLYKRYPSLWRRLATVEERKKIVASSHGKKTKPNTKDHGYTTLATSVTLLKASEVEEILDGNDEKYKAVSISTEPPTYLREQKAKRNSQWVPTLSNSSHHLDAVPCSTTINRNRMGRDKKRTFPLCFDDHDPAVIHENASQPEVLVPIRLDMEIDGQKLRDAFTWNMNEKLMTPEMFSEILCDDLDLNPLTFVPAIASAIRQQIESYPTDSILEDQSDQRVIIKLNIHVGNISLVDQFEWDMSEKENSPEKFALKLCSELGLGGEFVTTIAYSIRGQLSWHQKTYAFSENPLPTVEIAIRNTGDADQWCPLLETLTDAEMEKKIRDQDRNTRRMRRLANTGPAW

SEQ ID NO: 16 - protein, 1104 aa, Homo sapiens [sequence omitted]

SEQ ID NO: 17 - protein, 1213 aa, Homo sapiens
MAVRKKDGGPNVKYYEAADTVTQFDNVRLWLGKNYKKYIQAEPPTNKSLSSLVVQLLQFQEEVFGKHVSNAPLTKLPIKCFLDFKAGGSLCHILAAAYKFKSDQGWRRYDFQNPSRMDRNVEMFMTIEKSLVQNNCLSRPNIFLCPEIEPKLLGKLKDIIKRHQGTVTEDKNNASHVVYPVPGNLEEEEWVRPVMKRDKQVLLHWGYYPDSYDTWIPASEIEASVEDAPTPEKPRKVHAKWILDTDTFNEWMNEEDYEVNDDKNPVSRRKKISAKTLTDEVNSPDSDRRDKKGGNYKKRKRSPSPSPTPEVKEEKCKKGPSTPYTKSKRGHREEEQEDLTKDMDEPSPVPNVEEVTLPKTVNTKKDSESAPVKGGTMTDLDEQEDESMETTGKDEDENSTGNKGEQTKNPDLHEDNVTEQTHHIIIPSYAAWFDYNSVHAIERRALPEFFNGKNKSKTPEIYLAYRNFMIDTYRLNPQEYLTSTACRRNLAGDVCAISRVHAFLEQWGLINYQVDAESRPTPMGPPPTSHFHVLADTPSGLVPLQPKTPQQTSASQQMLNFPDKGKEKPTDMQNFGLRTDMYTKKNAPSKSKAAASATREWTEQETLLLLEALEMYKDDWNKVSEHVGSRTQDECILHFLRLPIEDPYLEDSEASLGPLAYQPIPFSQSGNPVMSTVAFLASVVDPRVASAAAKSALEEFSKMKEEVPTALVEAHVRKVEEAAKVTGKADPAFGLESSGIAGTTSDEPERIEESGNDEARVEGQATDEKKEPKEPREGGGAIEEEAKEKTSEAPKKDEEKGKEGDSEKESEKSDGDPIVDPEKEKEPKEGQEEVLKEVVESEGERKTKVERDIGEGNLSTAAAAALAAAAVKAKHLAAVEERKIKSLVALLVETQMKKLEIKLRHFEELETIMDREREALEYQRQQLLADRQAFHMEQLKYPEMRARQQHFQQMHQQQQQPPPALPPGSQPIPPTGAAGPPAVHGLAVAPASVVPAPAGSGAPPGSLGPSEQIGQAGSTRGPQQQQPAGAPQPGAVPPGVPPPGPHGPSPFPNQQTPPSMMPGAVPGSGHPGVAGNAPLGLPFGMPPPPPPPAPSIIPFGSLADSISINLPAPPNLMGSPPSPVRPGTLPPPNLPVSMANPLHPNLPATTTMPSSLPLGPGLGSAAAQSPAIVAAVQGNLLPSASPLPDPGTPLPPDPTAPSPGTVTPVPPPQ

SEQ ID NO: 18 - protein, 2285 aa, Homo sapiens [sequence omitted]

SEQ ID NO: 19 - protein, 1835 aa, Homo sapiens [sequence omitted]

SEQ ID NO: 20 - protein, 1582 aa, Homo sapiens
MGSKRRRATSPSSSVSGDFDDGHHSVSTPGPSRKRRRLSNLPTVDPIAVCHELYNTIRDYKDEQGRLLCELFIRAPKRRNQPDYYEVVSQPIDLMKIQQKLKMEEYDDVNLLTADFQLLFNNAKSYYKPDSPEYKAACKLWDLYLRTRNEFVQKGEADDEDDDEDGQDNQGTVTEGSSPAYLKEILEQLLEAIVVATNPSGRLISELFQKLPSKVQYPDYYAIIKEPIDLKTIAQRIQNGSYKSIHAMAKDIDLLAKNAKTYNEPGSQVFKDANSIKKIFYMKKAEIEHHEMAKSSLRMRTPSNLAAARLTGPSHSKGSLGEERNPTSKYYRNKRAVQGGRLSAITMALQYGSESEEDAALAAARYEEGESEAESITSFMDVSNPFYQLYDTVRSCRNNQGQLIAEPFYHLPSKKKYPDYYQQIKMPISLQQIRTKLKNQEYETLDHLECDLNLMFENAKRYNVPNSAIYKRVLKLQQVMQAKKKELARRDDIEDGDSMISSATSDTGSAKRKSKKNIRKQRMKILFNVVLEAREPGSGRRLCDLFMVKPSKRDYPDYYKIILEPMDLKIIEHNIRNDKYAGEEGMIEDMKLMFRNARHYNEEGSQVYNDAHILEKLLKEKRKELGPLPDDDDMASPKLKLSRKSGISPKKSKYMTPMQQKLNEVYEAVKNYTDKRGRRLSAIFLRLPSRSELPDYYLTIKKPMDMEKIRSHMMANKYQDIDSMVEDFVMMFNNACTYNEPESLIYKDALVLHKVLLETRRDLEGDEDSHVPNVTLLIQELIHNLFVSVMSHQDDEGRCYSDSLAEIPAVDPNFPNKPPLTFDIIRKNVENNRYRRLDLFQEHMFEVLERARRMNRTDSEIYEDAVELQQFFIKIRDELCKNGEILLSPALSYTTKHLHNDVEKERKEKLPKEIEEDKLKREEEKREAEKSEDSSGAAGLSGLHRTYSQDCSFKNSMYHVGDYVYVEPAEANLQPHIVCIERLWEDSAEKEVFKSDYYNKVPVSKILGKCVVMFVKEYFKLCPENFRDEDVFVCESRYSAKTKSFKKIKLWTMPISSVRFVPRDVPLPVVRVASVFANADKGDDEKNTDNSEDSRAEDNFNLEKEKEDVPVEMSNGEPVCHYFEQLHYNDMWLKVGDCVFIKSHGLVRPRVGRIEKVWVRDGAAYFYGPIFIHPEETEHEPTKMFYKKEVFLSNLEETCPMTCILGKCAVLSFKDFLSCKPTEIPENDILLCESRYNESDKQMKKFKGLKRFSLSAKVVDDEIYYFRKPIVPQKEPSPLLGKKIQLLEAKFAELEGGDDDIEEMGEEDSESTPKSAKGSAKKEGSKRKINMSGYILFSSEMRAVIKAQHPDYSFGELSRLVGTEWRNLETAKKAEYEGMMGGYPPGLPPLQGPVDGLVSMGSMQPLHPGGPPPHHLPPGVPGLPGIPPPGVMNQGVAPMVGTPAPGGSPYGQQVGVLGPPRQQAPPPYPGPHPAGPPVIQQPTTPMFVAPPPKTQRLLHSEAYLKYIEGLSAESNSISKWDQTLAARRRDVHLSKEQESRLPSHWLKSKGAHTTMADALWRLRDLMLRDTLNIRQAYNLENV

SEQ ID NO: 21 - protein, 746 aa, Homo sapiens
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKILEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFVDYRYSQADALKYVGIEREMEIP

SEQ ID NO: 22 - protein, 739 aa, Homo sapiens
MAPQKHGGGGGGGSGPSAGSGGGGFGGSAAVAAATASGGKSGGGSCGGGGSYSASSSSSAAAAAGAAVLPVKKPKMEHVQADHELFLQAFEKPTQIYRFLRTRNLIAPIFLHRTLTYMSHRNSRTNIKRKTFKVDDMLSKVEKMKGEQESHSLSAHLQLTFTGFFHKNDKPSPNSENEQNSVTLEVLLVKVCHKKRKDVSCPIRQVPTGKKQVPLNPDLNQTKPGNFPSLAVSSNEFEPSNSHMVKSYSLLFRVTRPGRREFNGMINGETNENIDVNEELPARRKRNREDGEKTFVAQMTVFDKNRRLQLLDGEYEVAMQEMEECPISKKRATWETILDGKRLPPFETFSQGPTLQFTLRWTGETNDKSTAPIAKPLATRNSESLHQENKPGSVKPTQTIAVKESLTTDLQTRKEKDTPNENRQKLRIFYQFLYNNNTRQQTEARDDLHCPWCTLNCRKLYSLLKHLKLCHSRFIFNYVYHPKGARIDVSINECYDGSYAGNPQDIHRQPGFAFSRNGPVKRTPITHILVCRPKRTKASMSEFLESEDGEVEQQRTYSSGHNRLYFHSDTCLPLRPQEMEVDSEDEKDPEWLREKTITQIEEFSDVNEGEKEVMKLWNLHVMKHGFIADNQMNHACMLFVENYGQKIIKKNLCRNFMLHLVSMHDFNLISIMSIDKAVTKLREMQQKLEKGESASPANEEITEEQNGTANGFSEINSKEKALETDSVSGVSKQSKKQKL

SEQ ID NO: 23 - protein, 441 aa, Homo sapiens
MSEREVSTAPAGTDMPAAKKQKLSSDENSNPDLSGDENDDAVSIESGTNTERPDTPTNTPNAPGRKSWGKGKWKSKKCKYSFKCVNSLKEDHNQPLFGVQFNWHSKEGDPLVFATVGSNRVTLYECHSQGEIRLLQSYVDADADENFYTCAWTYDSNTSHPLLAVAGSRGIIRIINPITMQCIKHYVGHGNAINELKFHPRDPNLLLSVSKDHALRLWNIQTDTLVAIFGGVEGHRDEVLSADYDLLGEKIMSCGMDHSLKLWRINSKRMMNAIKESYDYNPNKTNRPFISQKIHFPDFSTRDIHRNYVDCVRWLGDLILSKSCENAIVCWKPGKMEDDIDKIKPSESNVTILGRFDYSQCDIWYMRFSMDFWQKMLALGNQVGKLYVWDLEVEDPHKAKCTTLTHHKCGAAIRQTSFSRDSSILIAVCDDASIWRWDRLR

SEQ ID NO: 24 - protein, 1266 aa, Homo sapiens
MSKERPKRNIIQKKYDDSDGIPWSEERVVRKVLYLSLKEFKNSQKRQHAEGIAGSLKTVNGLLGNDQSKGLGPASEQSENEKDDASQVSSTSNDVSSSDFEEGPSRKRPRLQAQRKFAQSQPNSPSTTPVKIVEPLLPPPATQISDLSKRKPKTEDFLTFLCLRGSPALPNSMVYFGSSQDEEEVEEEDDETEDVKTATNNASSSCQSTPRKGKTHKHVHNGHVFNGSSRSTREKEPVQKHKSKEATPAKEKHSDHRADSRREQASANHPAAAPSTGSSAKGLAATHHHPPLHRSAQDLRKQVSKVNGVTRMSSLGAGVTSAKKMREVRPSPSKTVKYTATVTKGAVTYTKAKRELVKDTKPNHHKPSSAVNHTISGKTESSNAKTRKQVLSLGGASKSTGPAVNGLKVSGRLNPKSCTKEVGGRQLREGLQLREGLRNSKRRLEEAHQAEKPQSPPKKMKGAAGPAEGPGKKAPAERGLLNGHVKKEVPERSLERNRPKRATAGKSTPGRQAHGKADSASCENRSTSQPESVHKPQDSGKAEKGGGKAGWAAMDEIPVLRPSAKEFHDPLIYIESVRAQVEKFGMCRVIPPPDWRPECKLNDEMRFVTQIQHIHKLGRRWGPNVQRLACIKKHLKSQGITMDELPLIGGCELDLACFFRLINEMGGMQQVTELKKWNKLSDMLRIPKTAQERLAKLQEAYCQYILSYDSLSPEEHRRLEKEVLMEKEILEKRKGPLEGHTENDHHKFHPLPRLEPKNGLIHGVAPRNGFRSKLKEVGQAQLKTGRRRLFAQEKEVVKEEEEDKGVLNDFHKCIYKGRSVSLTTFYRTARNIMSMCFSKEPAPAEIEQEYWRLVEEKDCHVAVHCGKVDTNTHGSGFPVGKSEPFSRHGWNLTVLPNNTGSILRHLGAVPGVTIPWLNIGMVFSTSCWSRDQNHLPYIDYLHTGADCIWYCIPAEEENKLEDVVHTLLQANGTPGLQMLESNVMISPEVLCKEGIKVHRTVQQSGQFVVCFPGSFVSKVCCGYSVSETVHFATTQWTSMGFETAKEMKRRHIAKPFSMEKLLYQIAQAEAKKENGPTLSTISALLDELRDTELRQRRQLFEAGLHSSARYGSHDGSSTVADGKKKPRKWLQLETSERRCQICQHLCYLSMVVQENENVVFCLECALRHVEKQKSCRGLKLMYRYDEEQIISLVNQICGKVSGKNGSIENCLHKPTPKRGPRKRATVDVPPSRAVSLQFIQKCFELHHEDAQRPWSIYIYFFVIIIF

SEQ ID NO: 25 - RNA, 23 nt, Homo sapiens
aagaccccaccaaaacguccagg

SEQ ID NO: 26 - RNA, 23 nt, Homo sapiens
uggggucuuuauccgcucagcgg

SEQ ID NO: 27 - DNA, 20 bp, Homo sapiens
gtaggcaggcctttaggcaa

SEQ ID NO: 28 - DNA, 20 bp, Homo sapiens
gccggacatcccgaacttta

SEQ ID NO: 29 - DNA, 21 bp, Homo sapiens
aaccacgtgaggcatccaggc

SEQ ID NO: 30 - DNA, 21 bp, Homo sapiens
tcgtcgagcaatcatttggtt

SEQ ID NO: 31 - DNA, 21 bp, Homo sapiens
ttcgattccacagtgatcctg

SEQ ID NO: 32 - DNA, 21 bp, Homo sapiens
ttgtaggtcgggctgtagcca

SEQ ID NO: 33 - DNA, 21 bp, Homo sapiens
tagttgaccagctcatccgac

SEQ ID NO: 34 - DNA, 20 bp, Homo sapiens
gccggcgccattctatccgc

SEQ ID NO: 35 - DNA, 20 bp, Homo sapiens
ggcatgcgagaatctcacgc

SEQ ID NO: 36 - DNA, 20 bp, Homo sapiens
aagaccccaccaaaacgtcc

SEQ ID NO: 37 - DNA, 20 bp, Homo sapiens
tggggtctttatccgctcag

SEQ ID NO: 38 - DNA, 19 bp, Homo sapiens
gacagctctactgtatgcg

SEQ ID NO: 39 - DNA, 19 bp, Homo sapiens
ctctcaccaagacgccgag

(19) United States
(12) Patent Application Publication
(10) Pub. No.: US 2021/0338684 A1
(43) Pub. Date: Nov. 4, 2021

(54) DIAGNOSTIC AND THERAPEUTIC METHODS FOR CANCER

(71) Applicant: Genentech, Inc., South San Francisco, CA (US)

(72) Inventors: Robert L. YAUCH, Redwood City, CA (US); Xiaofen YE, Foster City, CA (US); Thomas E. JANUARIO, San Francisco, CA (US)

(21) Appl. No.: 17/370,721

(22) Filed: Jul. 8, 2021

Related U.S. Application Data
(63) Continuation of application No. 16/305,708, filed on Nov. 29, 2018.
(60) Provisional application No. 62/347,436, filed on Jun. 8, 2016.

(51) Int. Cl. A61K 31/5377; A61P 35/00; A61K 45/06; C12Q 1/6886

(52) U.S. Cl. CPC A61K 31/5377; A61P 35/00; A61K 45/06; C12Q 2600/154; C12Q 2600/106; C12Q 2600/136; C12Q 2600/158; C12Q 1/6886

(57) ABSTRACT

The present invention provides diagnostic and therapeutic methods for cancer. The invention provides methods of determining whether a patient having a cancer is likely to respond to treatment comprising an inhibitor of H3K27 methylation, methods of predicting responsiveness of a patient having a cancer to treatment comprising one or more inhibitors of H3K27 methylation, methods of selecting a therapy for a patient having a cancer, and methods of treating cancer based on expression levels of biomarkers of the invention (e.g., the expression level of SMARCA2 or the occupancy level of H3K27 at a SMARCA2 promoter).

### SEQUENCE LISTING

The instant application contains a Sequence Listing which has been submitted electronically in ASCII format and is hereby incorporated by reference in its entirety. Said ASCII copy, created on Jul. 2, 2021, is named 50474-141003_Sequence_Listing_7.2.21_ST25 and is 201,393 bytes in size.

### FIELD OF THE INVENTION

The present invention is directed to diagnostic and therapeutic methods for the treatment of proliferative cell disorders (e.g., cancers) using inhibitors of H3K27 methylation. Also provided are related kits and compositions.

### BACKGROUND OF THE INVENTION

Cancer remains one of the most deadly threats to human health. Certain cancers can metastasize and grow rapidly in an uncontrolled manner, making timely detection and treatment extremely difficult. In the U.S., cancer affects nearly 1.3 million new patients each year and is the second leading cause of death after heart disease, accounting for approximately one in four deaths.

Approximately 20% of human cancers are associated with somatic mutations in subunits of the SWI/SNF complex, a chromatin remodeling complex that influences gene regulation by disrupting histone-DNA contacts. SWI/SNF complexes are made up of approximately 12 subunits, consisting of two mutually exclusive catalytic ATPase subunits, SMARCA4 (BRG1) and SMARCA2 (BRM); several additional core complex members, including SMARCB1 (SNF5, SMARCC1, and SMARCC2; and subunits that are exclusive to two varieties of the SWI/SNF complex (i.e., the ARID1A subunits of the BAF complex and the ARID2 and PBRM1 subunits of the PBAF complex). In general, the mechanisms underlying tumorigenesis caused by specific SWI/SNF mutations have not been characterized.

An antagonist of the SWI/SNF complex, the polycomb repressive group 2 (PRC2) complex, contains the histone methyltransferase EZH2, which is involved in transcriptional silencing through methylation of lysine 27 at histone 3 (H3K27). In some cases, targeting EZH2 can provide an anti-tumor benefit, although associated diagnostic biomarkers are lacking.

Thus, there remains a need to develop improved methods for diagnosing and treating patient populations best suited for treatment including one or more inhibitors of H3K27 methylation (e.g., EZH2 inhibitors).

### SUMMARY OF THE INVENTION

The present invention provides diagnostic and therapeutic methods, kits, and compositions for the treatment of proliferative cell disorders (e.g., cancers).

In one aspect, the invention features a method of identifying a patient having a cancer who may benefit from treatment comprising one or more inhibitors of histone 3 lysine 27 (H3K27) methylation, the method comprising determining an expression level of SMARCA2 in a sample obtained from the patient, wherein a decreased expression level of SMARCA2 in the sample as compared to a reference expression level identifies the patient as one who may benefit from treatment comprising one or more inhibitors of H3K27 methylation.

In another aspect, the invention features a method of optimizing therapeutic efficacy for treatment of a patient having a cancer, the method comprising determining an expression level of SMARCA2 in a sample obtained from the patient, wherein a decreased expression level of SMARCA2 in a sample as compared to a reference expression level indicates that the patient has an increased likelihood of benefiting from treatment comprising one or more inhibitors of H3K27 methylation.

In another aspect, the invention features a method of predicting responsiveness of a patient having a cancer to treatment comprising one or more inhibitors of H3K27 methylation, the method comprising determining an expression level of SMARCA2 in a sample obtained from the patient, wherein a decreased expression level of SMARCA2 in the sample as compared to a reference expression level indicates that the patient has an increased likelihood of benefiting from treatment comprising one or more inhibitors of H3K27 methylation.

In another aspect, the invention features a method of selecting a treatment for a patient having a cancer, the method comprising determining an expression level of SMARCA2 in a sample obtained from the patient, wherein a decreased expression level of SMARCA2 in the sample as compared to a reference expression level indicates that the patient has an increased likelihood of benefiting from treatment comprising one or more inhibitors of H3K27 methylation.

In some embodiments of any of the preceding aspects, the expression level of SMARCA2 in a sample obtained from a patient is decreased by at least about 10% relative to the reference level. In some embodiments, the expression level of SMARCA2 in a sample obtained from a patient is decreased by at least about 25% relative to the reference level. In some embodiments, the expression level of SMARCA2 in a sample obtained from a patient is decreased by at least about 50% relative to the reference level. In some embodiments, the expression level of SMARCA2 in a sample obtained from a patient is decreased by at least about 75% relative to the reference level. In some embodiments, the expression level of SMARCA2 in a sample obtained from a patient is decreased by at least about 90% relative to the reference level. The expression level of SMARCA2 can be a median expression level or a mean expression level. In some embodiments, the reference expression level is selected from the group consisting of (i) the expression level of SMARCA2 in a sample obtained from the patient at a previous time point; (ii) the expression level of SMARCA2 in a reference population; or (iii) a pre-assigned expression level for SMARCA2. The reference expression level of SMARCA2 can be a median expression level or a mean expression level.

In some embodiments of any of the preceding aspects, the expression level of SMARCA2 is an mRNA expression level. In some embodiments, the mRNA expression level is determined by RNA-Seq, PCR, qPCR, RT-PCR, in situ hybridization, gene expression profiling, serial analysis of gene expression, or microarray analysis. In some embodiments, the mRNA expression level is determined by RNA-Seq. In some embodiments, the mRNA expression level is determined by qPCR. In some embodiments, the expression level is a protein expression level. In some embodiments, the protein expression level is determined using a method selected from the group consisting of immunohistochemistry (IHC), immunofluorescence, mass spectrometry, flow cytometry, and Western blot. In some embodiments, the protein expression level is determined by IHC.

In some embodiments of any of the preceding methods, the expression level of SMARCA2 in a sample obtained from the patient is decreased relative to the reference level and the method further comprises administering to the patient a therapeutically effective amount of one or more inhibitors of H3K27 methylation. In some embodiments, the administering of the one or more inhibitors of H3K27 methylation is after the determining of the expression level of SMARCA2. In other embodiments, the administering of the one or more inhibitors of H3K27 methylation is before the determining of the expression level of SMARCA2.

In another aspect, the invention features a method of treating a patient having a cancer, the method comprising administering to the patient a therapeutically effective amount of one or more inhibitors of H3K27 methylation, wherein the expression level of SMARCA2 in a sample obtained from the patient has been determined to be decreased as compared to a reference expression level.

In some embodiments of any of the preceding methods, the invention further includes determining an occupancy level of H3K27 (e.g., H3K27 trimethylation (H3K27me3)) at a SMARCA2 promoter in a sample obtained from the patient. An occupancy level can be methylation (e.g., mono-methylation, di-methylation, or tri-methylation) of H3K27 (e.g., H3K27me3) at a SMARCA2 promoter in a sample obtained from the patient.

In another embodiment, the invention features a method of identifying a patient having a cancer who may benefit from treatment comprising one or more inhibitors of H3K27 methylation, the method comprising determining an occupancy level of H3K27 (e.g., H3K27me3) at a SMARCA2 promoter in a sample obtained from the patient, wherein an increased occupancy level of H3K27 (e.g., H3K27me3) at the SMARCA2 promoter as compared to a reference occupancy level identifies the patient as one who may benefit from treatment comprising one or more inhibitors of H3K27 methylation.

In another embodiment, the invention features a method of optimizing therapeutic efficacy for treatment of a patient having a cancer, the method comprising determining an occupancy level of H3K27 (e.g., H3K27me3) at a SMARCA2 promoter in a sample obtained from the patient, wherein an increased occupancy level of H3K27 (e.g., H3K27me3) at the SMARCA2 promoter as compared to a reference occupancy level indicates that the patient has an increased likelihood of benefiting from treatment comprising one or more inhibitors of H3K27 methylation.

In another embodiment, the invention features a method of predicting responsiveness of a patient having a cancer to treatment comprising one or more inhibitors of H3K27 methylation, the method comprising determining an occupancy level of H3K27 (e.g., H3K27me3) at a SMARCA2 promoter in a sample obtained from the patient, wherein an increased occupancy level of H3K27 (e.g., H3K27me3) at the SMARCA2 promoter as compared to a reference occupancy level indicates that the patient has an increased likelihood of benefiting from treatment comprising one or more inhibitors of H3K27 methylation.

In another embodiment, the invention features a method of selecting a treatment for a patient having a cancer, the method comprising determining an occupancy level of H3K27 (e.g., H3K27me3) at a SMARCA2 promoter in a sample obtained from the patient, wherein an increased occupancy level of H3K27 (e.g., H3K27me3) at the SMARCA2 promoter as compared to a reference occupancy level indicates that the patient has an increased likelihood of benefiting from treatment comprising one or more inhibitors of H3K27 methylation.

In some embodiments, the occupancy level of H3K27 (e.g., H3K27me3) in a sample obtained from a patient is increased by at least about 10% relative to the reference occupancy level. In some embodiments, the occupancy level of H3K27 (e.g., H3K27me3) in a sample obtained from a patient is increased by at least about 50% relative to the reference occupancy level. In some embodiments, the occupancy level of H3K27 (e.g., H3K27me3) in a sample obtained from a patient is increased by at least about 100% relative to the reference occupancy level. In some embodiments, the occupancy level of H3K27 (e.g., H3K27me3) in a sample obtained from a patient is increased by at least about 500% relative to the reference occupancy level. In some embodiments, the occupancy level of H3K27 (e.g., H3K27me3) in a sample obtained from a patient is increased by at least about 1,000% relative to the reference occupancy level. The occupancy level of H3K27 (e.g., H3K27me3) at the SMARCA2 promoter can be a median expression level or a mean expression level. In some embodiments, the reference occupancy level is selected from the group consisting of (i) an occupancy level of H3K27 (e.g., H3K27me3) at a SMARCA2 promoter in a sample obtained from the patient at a previous time point; (ii) an occupancy level of H3K27 (e.g., H3K27me3) at a SMARCA2 promoter in a reference population; or (iii) a pre-assigned occupancy level of H3K27 (e.g., H3K27me3) at a SMARCA2 promoter. The reference occupancy level of H3K27 (e.g., H3K27me3) at the SMARCA2 promoter can be a median expression level or a mean expression level. The reference occupancy level of H3K27 (e.g., H3K27me3) at the SMARCA2 promoter can be determined by ChIP-seq or ChIP-PCR.

In some embodiments, the occupancy level of H3K27 (e.g., H3K27me3) at the SMARCA2 promoter is increased relative to the reference occupancy level and the method further comprises administering to the patient a therapeutically effective amount of one or more inhibitors of H3K27 methylation. In some embodiments, the administering of the one or more inhibitors of H3K27 methylation is after the determining of the occupancy level of H3K27 (e.g., H3K27me3) at the SMARCA2 promoter. In some embodiments, the administering of the one or more inhibitors of H3K27 methylation is before the determining of the occupancy level of H3K27 (e.g., H3K27me3) at the SMARCA2 promoter.

In another aspect, the invention features a method of treating a patient having a cancer, the method comprising administering to the patient a therapeutically effective amount of one or more inhibitors of H3K27 methylation, wherein the occupancy level of H3K27 (e.g., H3K27me3) at the SMARCA2 promoter in a sample obtained from the patient has been determined to be increased as compared to a reference occupancy level. In some embodiments, the method further includes determining an expression level of SMARCA2 in a sample obtained from the patient.

In some embodiments of any of the preceding aspects, the method further includes identifying a mutation in one or more genes encoding a nucleosome remodeling protein. In some embodiments, the nucleosome remodeling protein is a SWI/SNF family protein. In some embodiments, the SWI/SNF family protein is BRG1, SNF5 SWI/SNF complex 155-kDa subunit, SWI/SNF complex 170-kDa subunit, BAF, zipzap protein, or BAF180. In some embodiments, the one or more genes encoding a SWI/SNF family protein are selected from the group consisting of SMARCA4, SMARCB1, SMARCC1, SMARCC2, ARID1A, ARID2, and PBRM1.

In some embodiments of any of the preceding aspects, the sample obtained from the patient is a cell sample, a tissue sample, a whole blood sample, a plasma sample, or a serum sample. In some embodiments, the sample is a tumor cell sample. In some embodiments, the sample is a tumor tissue sample.

In some embodiments of any of the preceding aspects, the cancer comprises a mutation in one or more genes encoding a SWI/SNF family protein (e.g., a cancer associated with or characterized by a mutation in one or more genes encoding a SWI/SNF family protein). In some embodiments, the one or more genes encoding a SWI/SNF family protein are selected from the group consisting of SMARCA4, SMARCB1, SMARCC1, SMARCC2, ARID1A, ARID2, and PBRM1. In some embodiments, the cancer comprises a mutation in one or more of SMARCA4, SMARCB1, or ARID1A.

In some embodiments of any of the preceding aspects, the cancer is selected from the group consisting of an ovarian cancer (e.g., a small cell carcinoma of the ovary, e.g., a small cell carcinoma of the ovary, hypercalcemic type), a lung cancer, a gastric cancer, a bladder cancer, a breast cancer, a skin cancer, a colorectal cancer, a stomach cancer, a lymphoid cancer, a cervical cancer, a peritoneal cancer, a pancreatic cancer, a glioblastoma, a liver cancer, a bladder cancer, a colon cancer, a rectal cancer, an endometrial cancer, a uterine cancer, a salivary gland cancer, a renal cancer, a prostate cancer, a vulval cancer, a thyroid cancer, an anal cancer, a penile cancer, and a head and neck cancer. In some embodiments, the cancer is an ovarian cancer. In some embodiments, the ovarian cancer is an ovarian clear cell carcinoma. In some embodiments, the ovarian cancer is a small cell carcinoma of the ovary, e.g., a small cell carcinoma of the ovary, hypercalcemic type. In some embodiments, the cancer is a lung cancer. In some embodiments, the cancer is a gastric cancer. In some embodiments, the cancer is a bladder cancer. In some embodiments, the cancer is a rhabdoid cancer. In some embodiments, the rhabdoid cancer is a renal cancer or a brain cancer. In some embodiments, the rhabdoid cancer is a malignant rhabdoid cancer. In some embodiments, the malignant rhabdoid cancer is a SMARCB1-mutant malignant rhabdoid cancer.

In some embodiments of any of the preceding aspects, the one or more inhibitors of H3K27 methylation comprise an inhibitor of H3K27 methylation. In some embodiments, the inhibitor of H3K27 methylation is an EZH2 inhibitor. In some embodiments, the EZH2 inhibitor is a small molecule. In some embodiments, the EZH2 inhibitor is selected from the group consisting of EPZ-6438, CPI-169, CPI-1205, EPZ005687, GSK-126, GSK343, and GSK503. In some embodiments, the EZH2 inhibitor is EPZ-6438. In some embodiments, the EZH2 inhibitor is CPI-169. In some embodiments, the EZH2 inhibitor is CPI-1205.

In some embodiments, the one or more inhibitors of H3K27 methylation disrupt the formation or activity of polycomb repressive complex 2 (PRC2). In some embodiments, the one or more inhibitors of H3K27 methylation comprise a SUZ12 antagonist, an EED antagonist, or a jumonji antagonist.

In some embodiments, the method includes administering to the patient a first inhibitor of H3K27 methylation and a second inhibitor of H3K27 methylation. In some embodiments, the first inhibitor of H3K27 methylation and the second inhibitor of H3K27 methylation are co-administered. In other embodiments, the first inhibitor of H3K27 methylation and the second inhibitor of H3K27 methylation are sequentially administered.

In some embodiments, the method includes administering to the patient an additional therapeutic agent. In some embodiments, the additional therapeutic agent is an anti-cancer agent. In some embodiments, the additional therapeutic agent and the one or more inhibitors of H3K27 methylation are co-administered. In some embodiments, the additional therapeutic agent and the one or more inhibitors of H3K27 methylation are sequentially administered. In some embodiments, the anti-cancer agent is selected from the group consisting of a chemotherapeutic agent, a growth inhibitory agent, a cytotoxic agent, an agent used in radiation therapy, an anti-angiogenesis agent, an apoptotic agent, an anti-tubulin agent, and an immunotherapy agent. In some embodiments, the anti-cancer agent is a chemotherapeutic agent.

In another aspect, the invention features a composition comprising one or more inhibitors of H3K27 methylation for use in a method of treating a patient suffering from a cancer, wherein a sample obtained from the patient has been determined to have a decreased expression level of SMARCA2 in a sample as compared to a reference expression level.

In another aspect, the invention features a composition comprising one or more inhibitors of H3K27 methylation for use in a method of treating a patient suffering from a cancer, wherein a sample obtained from the patient has been determined to have an increased occupancy level of H3K27 (e.g., H3K27me3) at a SMARCA2 promoter in a sample as compared to a reference occupancy level.

In another aspect, the invention features a kit for identifying a patient who may benefit from treatment comprising one or more inhibitors of H3K27 methylation, the kit comprising: (a) polypeptides or polynucleotides capable of determining an expression level of SMARCA2 in a sample; and (b) instructions for using the polypeptides or polynucleotides to identify a patient that may benefit from treatment comprising one or more inhibitors of H3K27 methylation.

In another aspect, the invention features a kit for identifying a patient who may benefit from treatment comprising one or more inhibitors of H3K27 methylation, the kit comprising: (a) reagents capable of determining an occupancy level of H3K27 (e.g., H3K27me3) at a SMARCA2 promoter in a sample; and (b) instructions for using the reagents to identify a patient that may benefit from treatment comprising one or more inhibitors of H3K27 methylation.

In some embodiments of any of the preceding aspects, the patient is a human patient.

### BRIEF DESCRIPTION OF THE DRAWINGS

FIG. 1A is a schematic plate diagram showing the experimental setup used in FIGS. 1B and 1C. The concentration of EPZ-6438 is labeled in each of the wells.

FIG. 1B is a pair of photographs of plates showing colony formation of control (i.e., non-SMARCA4-mutant cells) in response to increasing doses of EPZ-6438.

FIG. 1C is a series of photographs of plates showing colony formation of EPZ-6438-sensitive and EPZ-resistant SMARCA4-mutant cells in response to increasing doses of EPZ-6438. TOV-112D and COV434 are ovarian cancer cell lines; SNU-484 is a gastric cancer cell line; NCI-H1703, NCI-H522, NCI-H661, H1299, A549, NCI-H1568, and HCC-15 are lung cancer cell lines; and UM-UC-3 is a bladder cancer cell line. SNF5 mutant G401 cells and ARID1A mutant A2780 cells are used as controls.

FIG. 2A is a schematic plate diagram showing the experimental setup used in FIGS. 2B and 2C. The concentration of drug is labeled in each of the wells.

FIG. 2B is a series of photographs showing colony formation of EPZ-6438-sensitive cell lines, SNU-484 and TOV112D, in response to increasing doses of various EZH2 inhibitors: EPZ-6438, CPI-169, and GSK126. The histone deacetylase inhibitor, trichostatin A (TSA) was used as a positive control.

FIG. 2C is a series of photographs showing colony formation of EPZ-6438-resistant cell lines, H1299 and A549, in response to increasing doses of various EZH2 inhibitors: EPZ-6438, CPI-169, and GSK126. The histone deacetylase inhibitor, trichostatin A (TSA) was used as a positive control.

FIG. 3A is a series of photographs showing the effects of genetic deletion of EZH2 by CRISPR on protein expression and colony formation by EPZ-6438-resistant cell lines: RMG, ES-2, OVISE, H1299, and A549. Western blotting was carried out on lysates collected at an early (1 week) and a later (2 weeks) time point following infection with lentivirus guide RNAs targeting EZH2 or luciferase (gLuc).

FIG. 3B is a series of photographs showing the effects of genetic deletion of EZH2 by CRISPR on protein expression and colony formation by EPZ-6438-sensitive cell lines: TOV-21G and TOV-112D. Western blotting was carried out on lysates collected at an early (1 week) and a later (2 weeks) time point following infection with lentivirus guide RNAs targeting EZH2 or luciferase (gLuc).

FIG. 4A is a series of photomicrographs showing morphological changes of indicated cell lines following 21 days of treatment with 5 μM EPZ-6438 or DMSO control.

FIG. 4B is a series of bar graphs showing caspase 3/7 activation upon treatment of indicated cell lines with increasing doses of EPZ-6438 (0 μM, 0.74 μM, 2.2 μM, and 6.7 μM) after 7 days and 13 days of treatment. Data are presented as an average fold change (fc) in caspase 3/7 fluorescent cell counts relative to DMSO control across triplicate samples. Error bars represent standard deviation.

FIG. 4C is a series of fluorescent images of active caspase 3/7 positive cells following 7 days of treatment with the indicated concentration of EPZ-6438.

FIG. 4D is a series of photomicrographs showing staining for β-galactosidase in representative SMARCA4-mutant cell lines.

FIG. 4E is a bar graph showing EPZ-6438-mediated inhibition of DNA synthesis as measured by 5-ethynyl-2′-deoxyuridine (Edu) incorporation in NCI-H522 cells following 8 weeks of treatment, relative to an EPZ-6438 resistance cell line, NCI-H1568. Gray bars represent a DMSO control. Black bars represent EPZ-6438 treatment.

FIG. 4F is a graph showing the dose-dependent inhibition of in vivo growth of NCI-H522 xenografts following twice-daily oral administration of EPZ-6438 treatment for 23 days. Solid circles represent the vehicle control, squares represent a dosage of 30 mg/kg, triangles represent a dosage of 100 mg/kg, and open circles indicate a dosage of 450 mg/kg. Data are presented as cubic regression splines of tumor volumes over time.

FIG. 4G is a series of western blots showing H3K27 methylation as a result of target inhibition in NCI-H522 tumor xenograft tissue collected from a cohort of animals at day 7, three hours following twice-daily oral administration of the indicated doses of EPZ-6438.

FIG. 5 is a series of immunoblots showing the expression of various modified histones, as well as EZH2 and SUZ12, by EPZ-6438-sensitive cells and EPZ-6438-resistant cells. Histone 3 (H3) served as a positive control.

FIG. 6 is a series of immunoblots showing the effect of an increasing dose of EPZ-6438 on expression of modified histones (mono-, di-, and tri-methylated forms of H3K27) by EPZ-6438-sensitive cells and EPZ-6438-resistant cells following a 6-day treatment.

FIG. 7 is a supervised analysis graph of genes that are most differentially expressed between EPZ-6438 sensitive (n=6) and resistant (n=5) SMARCA4-mutant models (log 2 fold change >1, p≤0.05). Expression estimates are reported as z-scores derived from log 2 rpkm (reads per kilobase per million mapped reads).

FIG. 8 is a series of immunoblots showing the protein expression of various SWI/SNF complex members by EPZ-6438-sensitive cells and EPZ-6438-resistant cells. The ARID1A-mutant A2780 cell line served as a control for SMARCA4 immunoblotting.

FIG. 9 is a bar graph showing the expression of SMARCA2 mRNA by EPZ-6438-sensitive cells and EPZ-6438-resistant cells at baseline (black dots) and in response to EPZ-6438 treatment after 6 days (solid bars) and 10 days (open bars).

FIG. 10A is a genome viewer graph showing binding of the SMARCA2 promoter by H3K27me3 in the EPZ-6438-sensitive SNU-484 and TOV-112D cell lines, but not in the EPZ-6438-resistant H1299 cell line.

FIG. 10B is an expanded view of the SMARCA2 promoter region showing binding by H3K27me3 in TOV-112D cells relative to H1299 cells.

FIG. 11 is a graph showing results of a quantitative PCR analysis of H3K27me3 ChIP DNA enrichment at three locations in the SMARCA2 gene promoter (circles=chr9:2015841-2015938; squares=chr9:2016847-2016917; and triangles=chr9:2016214-201633) and a control region (actin promoter) across SMARCA4-mutant cancer cell lines. The y-axis represents average enrichment of the region in the H3K27me3 IP as a percentage of the level observed in the input lysate. Error bars indicate standard deviation of the mean estimated from two independent immunoprecipitations.

FIG. 12 is a bar graph showing ChIP-PCR readouts of H3K27 trimethylation (H3K27me3) at the SMARCA2 promoter (black bars) relative to control regions (gray and white bars) in the EPZ-6438-resistant H1299 cell line and in the EPZ-6438-sensitive TOV-112D cell line. A control IgG immunoprecipitation and PCR for two gene regions devoid of HeK27me3 serve as controls.

FIG. 13 is a bar graph showing ChIP-PCR readouts of H3K27me3 at the SMARCA2 promoter and two control regions in response to DMSO (white bars) or EPZ-6438 (black bars) in TOV-112D cells.

FIG. 14A is a set of photographs of plates showing colony formation in response to EPZ-6438 by wildtype cells that have undergone SMARCA2 (BRM) genetic knockout.

FIG. 14B is a set of immunoblots showing the expression of SMARCA2 protein relative to histone 3 controls by the cells of FIG. 10A.

FIG. 15A is an immunoblot showing the effect of doxycycline on expression of helicase in the insoluble nuclear fraction. Following treatment with 0.5 μg/mL doxycycline for four days, cells were fractioned for the cytosolic fraction, the soluble nuclear fraction, and the insoluble nuclear fraction. GAPDH serves as a control for the cytosolic fraction, H3 serves as a control for the insoluble nuclear fraction, and PARP serves as a control for the soluble and insoluble nuclear fractions.

FIG. 15B is an immunoblot showing the results of SMARCC1 immunoprecipitations for SMARCA2 or SMARCA4, showing that the doxycycline-induced helicase can re-associate with the core SWI/SNF complex protein.

FIG. 16A is a scatterplot showing log 2 fold expression change estimates for all genes following doxycycline (dox)-inducible expression of SMARCA2 (x-axis) and SMARCA4 (y-axis) in TOV-112D cells. The sets of genes significantly differentially expressed following induction of either helicase significantly overlap (P<2e-16, Fisher's Exact Test). Genes non-specifically impacted by dox treatment in vector control TOV-112D cells are filtered from this analysis.

FIG. 16B is a Venn diagram depicting the overlap between genes significantly differentially expressed (log 2 fc 1, p<0.05) following dox-induced expression of SMARCA4 or SMARCA2, or treatment with 1 μM EPZ-6438 (+EZH2i) in TOV-112D cells.

FIG. 17A is a series of immunoblots showing the effect of various doses of EPZ-6438 on the expression of modified histones in the EPZ-6438-sensitive cell line, G401, following the expression of a shRNA targeting SMARCA2 (shBRM) or a non-targeting control (shNTC).

FIG. 17B is a series of photographs of plates showing colony formation of G401 cells in response to increasing concentrations of EPZ-6438 in cells expressing shNTC or shBRM.

FIG. 17C is a series of immunoblots showing the effect of various doses of EPZ-6438 on the expression of H3K27me3 in the EPZ-6438-sensitive cell line, COV434, following the expression of a shRNA targeting SMARCA2 (shBRM) or a non-targeting control (shNTC).

FIG. 17D is a set of photographs of plates showing colony formation of COV434 cells in response to increasing concentrations of EPZ-6438 in cells expressing shNTC or shBRM.

FIG. 17E is a series of immunoblots showing the effect of various doses of EPZ-6438 on the expression of H3K27me3 in the EPZ-6438-sensitive cell line, SNU-484, following the expression of a shRNA targeting SMARCA2 (shBRM) or a non-targeting control (shNTC).

FIG. 17F is a set of photographs of plates showing colony formation of SNU-484 cells in response to increasing concentrations of EPZ-6438 in cells expressing shNTC or shBRM.

FIG. 18A is a series of immunoblots showing the effect of various doses of EPZ-6438 on the expression of modified histones in the EPZ-6438-sensitive cell line, TOV-112, following the expression of a shRNA targeting SMARCA2 (shBRM) or a non-targeting control (shNTC).

FIG. 18B is a series of photographs of plates showing colony formation of TOV-112D cells in response to increasing concentrations of EPZ-6438 in cells expressing shNTC or shBRM.

FIG. 18C is a graph showing a dose-dependent induction of caspase 3/7 activity in shBRM-treated TOV-112D cells relative to shNTC-treated TOV-112D cells in response to increasing concentrations of EPZ-6438.

FIG. 19A is a graph showing fold change (fc) in caspase 3/7 activity as a result of increasing concentrations of EPZ-6438 in three separate TOV-112D clones that have had SMARCA2 genetically ablated. Clones were generated from TOV-112D cells transfected with a vector expressing paired guide RNAs targeting SMARCA2. Ctrl-P indicates parental stable Cas9 cells, and gCtrl-1 and gCtrl-2 indicate clones exhibiting no SMARCA2 deletion.

FIG. 19B is a series of immunoblots showing the effect of EPZ-6438 on the clones from FIG. 19A, to confirm the ability of EPZ-6438 to induce SMARCA2 expression.

FIG. 19C is a scatterplot depicting the log 2 fold expression change estimates for all genes following treatment with 5 μM EPZ-6438 in TOV-112D cells that express a non-targeting shRNA (x-axis) or a SMARCA2-targeting shRNA (y-axis). Estimates are derived from three independent treatments per cell line.

FIG. 19D is a heatmap depicting Z-score normalized expression of EPZ-6438-induced genes that are significantly suppressed by SMARCA2 knockout or shRNA-mediated knockdown. shBRM and gBRM refer to shSMARCA2 or SMARCA2 guide RNAs, respectively.

FIG. 19E is a bar graph showing cathepsin B (CTSB) mRNA levels in TOV-112D cells expressing a stable shRNA targeting SMARCA2, and in clones engineered to genetically ablate SMARCA2 expression through CRISPR, following treatment with 5 μM EPZ-6438.

FIG. 19F is an immunoblot of TOV112D cells expressing a shRNA targeting SMARCA2 or three separate shRNAs targeting CTSB on expression of SMARCA2 and CTSB following treatment with EPZ-6438. H3K27me3 serves as a control for the EPZ-6438 treatment.

FIG. 19G is a graph showing caspase 3/7 activity in response to increasing concentrations of EPZ-6438, showing that expression of shRNAs targeting CTSB significantly suppressed the activation of caspase 3/7 upon treatment with EPZ-6438.

FIG. 20 is a series of fluorescent images showing colony formation of ARID1A-mutant cell lines relative to ARID1A-wildtype cell lines in response to EZH2 inhibitors: EPZ-6438 and CPI-169.

FIG. 21 is a series of photographs showing the effect of treatment with various doses of EPZ-6438 on clonogenic growth across a panel of ARID1A-mutant cancer cell lines, a subset of which are sensitive to EPZ-6438. The dosing scheme is identical to that shown in FIG. 1A.

FIG. 22 is series of immunoblots and photographs showing colony formation, which show that genetic ablation of EZH2 phenocopies the effect of EPZ-6438 on colony formation in ARID1A-mutant and wild-type cells. Cells stably expressing Cas9 were infected with lentivirus expressing guide RNAs targeting EZH2 (gEZH2-#4, #5) or luciferase (gLuc-#1, #2) as a negative control. Immunoblots for EZH2 and its substrate, H3K27me3, were performed on lysates collected at an early (1 week) and a late (2 week) time point following infection. Colony formation was imaged at the two week time point.

FIG. 23A is a series of bar graphs showing cell number, and corresponding photographs showing colony formation, which depict the effect of the EZH2 methyltransferase inhibitor, CPI-169, on colony formation of ARID1A-mutant ovarian cell lines. Colonies were stained using SYTO60 red fluorescent nucleic acid stain. For bar graphs, cells were counted from a parallel culture plate.

FIG. 23B is a series of bar graphs showing cell number, and corresponding photographs showing colony formation, which depict the effect of the EZH2 methyltransferase inhibitor, CPI-169, on colony formation of ARID1A-WT ovarian cell lines. Colonies were stained using SYTO60 red fluorescent nucleic acid stain. For bar graphs, cells were counted from a parallel culture plate.

FIG. 24 is a series of photographs showing the effect of EZH2 inhibition using EPZ-6438 or CPI-169 on acini formation in ARID1A-mutant cell lines, demonstrating the lack of activity in two ARID1A-mutant cell lines (OVTOKO and OVISE) that were additionally resistant to the effects of EPZ-6438 on clonogenic growth. A2780 cells serve as a positive control, demonstrating EZH2-mediated inhibition of both clonogenic growth and acini formation.

FIG. 25 is a graph showing in vivo tumor volume (mm³) over time in response to twice-daily administration of the indicated doses of EPZ-6438 for 28 days in TOV-21G xenografts. Data are presented as cubic regression splines of tumor volumes over time plotted on the natural scale.

FIG. 26 is a series of immunoblots detecting H3K27me3, which demonstrates target inhibition in TOV-21G tumor xenograft tissue collected from a cohort of animals at day 7, three hours following twice-daily oral administration of the indicated doses of EPZ-6438.

FIG. 27A is a graph showing the constitutive expression of SMARCA2 mRNA in EPZ-6438-sensitive, SNF5-mutated cells (dark circles); EPZ-6438-sensitive, SMARCA4-mutated cells (dark squares); EPZ-6438-sensitive, ARID1A-mutated cells (dark triangles); EPZ-6438-resistant, SMARCA4-mutated cells (light circles); EPZ-6438-resistant, ARID1A-mutated cells (light squares); and wildtype (WT) cells (light circles).

FIG. 27B is a graph showing the constitutive expression of SMARCA2 mRNA in EPZ-6438-sensitive cells versus EPZ-6438-resistant cells.

FIG. 28A is a graph showing the fold change of EZH2, SMARCA2, and TKTL1 expression levels by SMARCB1-mutant malignant rhabdoid tumor (MRT) cell line in response to 6 days of treatment with EPZ-6438 (5 μM) or the DNA methyltransferase inhibitor, 5-aza-2′-deoxycytidine (5-Aza; 1 μM). EZH2 mRNA is a negative control and TKTL1 mRNA is a control for 5-aza treatment.

FIG. 28B is a series of immunoblots showing the expression of SMARCA2 and H3K27me3 by SMARCB1-mutant MRT cells in response to EPZ-6438 (5 μM) or 5-Aza (1 μM).

FIG. 29 is a graph showing the fold change of SMARCA2 expression in EPZ-6438-sensitive and EPZ-6438-resistant ARID1A-mutant cell lines in response to 6 days of treatment with EPZ-6438 (5 μM; gray bars) relative to baseline expression (black dots). Hec-1A and SK-OV-3 are ARID1A-mutant cell lines that are insensitive to EPZ-6438 treatment. RMG-1 cells are ARID1A wildtype and insensitive to EPZ-6438.

### DETAILED DESCRIPTION OF THE INVENTION

### I. Introduction

The present invention provides diagnostic methods, therapeutic methods, and compositions for the treatment of proliferative cell disorders (e.g., cancer (e.g., rhabdoid cancer (e.g., malignant rhabdoid cancer, e.g., malignant rhabdoid brain cancer or malignant rhabdoid renal cancer)), ovarian cancer, lung cancer, gastric cancer, bladder cancer, breast cancer, skin cancer, colorectal cancer, stomach cancer, lymphoid cancer, cervical cancer, peritoneal cancer, pancreatic cancer, glioblastoma, liver cancer, bladder cancer, colon cancer, endometrial cancer, uterine cancer, renal cancer, prostate cancer, thyroid cancer, and head and neck cancer)). The invention is based, at least in part, on the discovery that SMARCA2 expression levels can be used as biomarkers (e.g., predictive biomarkers) in methods of predicting sensitivity to treatment including inhibitors of H3K27 methylation (e.g., EZH2 inhibitors); optimizing therapeutic efficacy for treatment including inhibitors of H3K27 methylation; selecting a therapy involving administration of inhibitors of H3K27 methylation for a patient having a cancer; and treating a patient having a cancer with a therapy including inhibitors of H3K27 methylation. In some instances, a decreased expression level (e.g., repression) of SMARCA2 may be used to predict responsiveness to treatment including inhibitors of H3K27 methylation. In other cases, an increased occupancy level of H3K27 (e.g., H3K27 trimethylation (H3K27me3)) at a SMARCA2 promoter may be used to predict responsiveness to treatment including inhibitors of H3K27 methylation. The invention also provides methods of using the expression levels or methylation status of SMARCA2 as prognostic biomarkers, because patients with low SMARCA2 expression can be expected to have a better response to inhibitors of H3K27 methylation than patients with higher SMARCA2 expression. Similarly, patients with high occupancy levels of H3K27 (e.g., H3K27me3) at a SMARCA2 promoter can be expected to have a better response to inhibitors of H3K27 methylation than patients with low occupancy levels.

### II. Definitions

It is to be understood that aspects and embodiments of the invention described herein include “comprising,” “consisting,” and “consisting essentially of” aspects and embodiments. As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) embodiments that are directed to that value or parameter per se.

A used herein, the terms “SWI/SNF complex protein” or “SWI/SNF family protein” are used interchangeably to refer to a member of the SWI/SNF (Switch/Sucrose Non-Fermentable) complex from any vertebrate source, including mammals such as primates (e.g., humans) and rodents (e.g., mice and rats), unless otherwise indicated. Exemplary SWI/SNF complex proteins are BRG1, SNF5 SWI/SNF complex 155 kDa subunit, SWI/SNF complex 170-kDa subunit, BAF, zipzap protein, and BAF180. Exemplary genes encoding a SWI/SNF family protein are SMARCA4, SMARCB1, SMARCC1, SMARCC2, ARID1A, ARID2, and PBRM1.

The term “SMARCA2,” as used herein, refers to any native SMARCA2 (SWI/SNF Related, Matrix Associated, Actin Dependent Regulator Of Chromatin, Subfamily A, Member 2) from any vertebrate source, including mammals such as primates (e.g., humans) and rodents (e.g., mice and rats), unless otherwise indicated. The term encompasses “full-length,” unprocessed SMARCA2 as well as any form of SMARCA2 that results from processing in the cell. The term also encompasses naturally occurring variants of SMARCA2, e.g., splice variants or allelic variants. The nucleic acid sequence of an exemplary human SMARCA2 is set forth in SEQ ID NO: 1. Human SMARCA2 encodes the protein, brahma homolog (BRM), an exemplary amino acid sequence of which is shown in SEQ ID NO: 13.

The term “SMARCA4,” as used herein, refers to any native SMARCA4 (SWI/SNF Related, Matrix Associated, Actin Dependent Regulator Of Chromatin, Subfamily A, Member 4) from any vertebrate source, including mammals such as primates (e.g., humans) and rodents (e.g., mice and rats), unless otherwise indicated. The term encompasses “full-length,” unprocessed SMARCA4 as well as any form of SMARCA4 that results from processing in the cell. The term also encompasses naturally occurring variants of SMARCA4, e.g., splice variants or allelic variants. The nucleic acid sequence of an exemplary human SMARCA4 is set forth in SEQ ID NO: 2. Human SMARCA4 encodes the protein, BRG1, an exemplary amino acid sequence of which is shown in SEQ ID NO: 14.

The term “SMARCB1,” as used herein, refers to any native SMARCB1 (SWI/SNF Related, Matrix Associated, Actin Dependent Regulator Of Chromatin, Subfamily B, Member 1) from any vertebrate source, including mammals such as primates (e.g., humans) and rodents (e.g., mice and rats), unless otherwise indicated. The term encompasses “full-length,” unprocessed SMARCB1 as well as any form of SMARCB1 that results from processing in the cell. The term also encompasses naturally occurring variants of SMARCB1, e.g., splice variants or allelic variants. The nucleic acid sequence of an exemplary human SMARCB1 is set forth in SEQ ID NO: 3. Human SMARCB1 encodes the protein, SNF5 (IN11), an exemplary amino acid sequence of which is shown in SEQ ID NO: 15.

The term “SMARCC1,” as used herein, refers to any native SMARCC1 (SWI/SNF Related, Matrix Associated, Actin Dependent Regulator Of Chromatin, Subfamily C, Member 1) from any vertebrate source, including mammals such as primates (e.g., humans) and rodents (e.g., mice and rats), unless otherwise indicated. The term encompasses “full-length,” unprocessed SMARCC1 as well as any form of SMARCC1 that results from processing in the cell. The term also encompasses naturally occurring variants of SMARCC1, e.g., splice variants or allelic variants. The nucleic acid sequence of an exemplary human SMARCC1 is set forth in SEQ ID NO: 4. Human SMARCC1 encodes the 155-kDa subunit of the SWI/SNF complex, an exemplary amino acid sequence of which is shown in SEQ ID NO: 16.

The term “SMARCC2,” as used herein, refers to any native SMARCC2 (SWI/SNF Related, Matrix Associated, Actin Dependent Regulator Of Chromatin, Subfamily C, Member 2) from any vertebrate source, including mammals such as primates (e.g., humans) and rodents (e.g., mice and rats), unless otherwise indicated. The term encompasses “full-length,” unprocessed SMARCC2 as well as any form of SMARCC2 that results from processing in the cell. The term also encompasses naturally occurring variants of SMARCC2, e.g., splice variants or allelic variants. The nucleic acid sequence of an exemplary human SMARCC2 is set forth in SEQ ID NO: 5. Human SMARCC2 encodes the 170-kDa subunit of the SWI/SNF complex, an exemplary amino acid sequence of which is shown in SEQ ID NO: 17.

The term “ARID1A,” as used herein, refers to any native ARID1A (AT Rich Interactive Domain 1A (SWI-Like)) from any vertebrate source, including mammals such as primates (e.g., humans) and rodents (e.g., mice and rats), unless otherwise indicated. The term encompasses “full-length,” unprocessed ARID1A as well as any form of ARID1A that results from processing in the cell. The term also encompasses naturally occurring variants of ARID1A, e.g., splice variants or allelic variants. The nucleic acid sequence of an exemplary human ARID1A is set forth in SEQ ID NO: 6. Human ARID1A encodes the protein, BAF250a, an exemplary amino acid sequence of which is shown in SEQ ID NO: 18.

The term “ARID2,” as used herein, refers to any native ARID2 (AT Rich Interactive Domain 2 (ARID, RFX-Like)) from any vertebrate source, including mammals such as primates (e.g., humans) and rodents (e.g., mice and rats), unless otherwise indicated. The term encompasses “full-length,” unprocessed ARID2 as well as any form of ARID2 that results from processing in the cell. The term also encompasses naturally occurring variants of ARID2, e.g., splice variants or allelic variants. The nucleic acid sequence of an exemplary human ARID2 is set forth in SEQ ID NO: 7. Human ARID2 encodes the zipzap protein, an exemplary amino acid sequence of which is shown in SEQ ID NO: 19.

The term “PBRM1,” as used herein, refers to any native PBRM1 (Polybromo 1) from any vertebrate source, including mammals such as primates (e.g., humans) and rodents (e.g., mice and rats), unless otherwise indicated. The term encompasses “full-length,” unprocessed PBRM1 as well as any form of PBRM1 that results from processing in the cell. The term also encompasses naturally occurring variants of PBRM1, e.g., splice variants or allelic variants. The nucleic acid sequence of an exemplary human PBRM1 is set forth in SEQ ID NO: 8. Human PBRM1 encodes the protein, BAF180, an exemplary amino acid sequence of which is shown in SEQ ID NO: 20.

A used herein, the term “PRC2,” as used herein, refers to a member of the PCR2 (polycomb repressive complex 2) from any vertebrate source, including mammals such as primates (e.g., humans) and rodents (e.g., mice and rats), unless otherwise indicated. Exemplary PRC2 proteins are EZH2, SUZ12, EED, and jumonji.

The term “EZH2,” as used herein, refers to any native EZH2 (Enhancer of zeste 2 Polycomb Repressive Complex 2) from any vertebrate source, including mammals such as primates (e.g., humans) and rodents (e.g., mice and rats), unless otherwise indicated. The term encompasses “full-length,” unprocessed EZH2 as well as any form of EZH2 that results from processing in the cell. The term also encompasses naturally occurring variants of EZH2, e.g., splice variants or allelic variants. The nucleic acid sequence of an exemplary human EZH2 is set forth in SEQ ID NO: 9. The amino acid sequence of an exemplary EZH2 protein encoded by a human EZH2 gene is shown in SEQ ID NO: 21.

The term “SUZ12,” as used herein, refers to any native SUZ12 (SUZ12 Polycomb Repressive Complex 2 Subunit) from any vertebrate source, including mammals such as primates (e.g., humans) and rodents (e.g., mice and rats), unless otherwise indicated. The term encompasses “full-length,” unprocessed SUZ12 as well as any form of SUZ12 that results from processing in the cell. The term also encompasses naturally occurring variants of SUZ12, e.g., splice variants or allelic variants. The nucleic acid sequence of an exemplary human SUZ12 is set forth in SEQ ID NO: 10. The amino acid sequence of an exemplary SUZ12 protein encoded by a human SUZ12 gene is shown in SEQ ID NO: 22.

The term “EED,” as used herein, refers to any native EED (Embryonic Ectoderm Development) from any vertebrate source, including mammals such as primates (e.g., humans) and rodents (e.g., mice and rats), unless otherwise indicated. The term encompasses “full-length,” unprocessed EED as well as any form of EED that results from processing in the cell. The term also encompasses naturally occurring variants of EED, e.g., splice variants or allelic variants. The nucleic acid sequence of an exemplary human EED is set forth in SEQ ID NO: 11. The amino acid sequence of an exemplary EED protein encoded by a human EED gene is shown in SEQ ID NO: 23.

The term “JARID2,” as used herein, refers to any native JARID2 (Jumonji, AT Rich Interactive Domain 2) from any vertebrate source, including mammals such as primates (e.g., humans) and rodents (e.g., mice and rats), unless otherwise indicated. The term encompasses “full-length,” unprocessed JARID2 as well as any form of JARID2 that results from processing in the cell. The term also encompasses naturally occurring variants of JARID2, e.g., splice variants or allelic variants. The nucleic acid sequence of an exemplary human JARID2 is set forth in SEQ ID NO: 12. Human JARID2 encodes the protein, jumonji, an exemplary amino acid sequence of which is shown in SEQ ID NO: 24.

As used herein, the term “Inhibitor of H3K27 methylation” refers to any inhibitor of H3K27 methylation that is currently known in the art or that will be identified in the future, and includes any chemical entity that, upon administration to a patient, results in inhibition of a biological activity associated with trimethylation of H3K27 in the patient. Such H3K27 inhibitors include but are not limited to low molecular weight inhibitors, antibodies or antibody fragments, antisense constructs, small inhibitory RNAs (i.e., RNA interference by dsRNA; RNAi), and ribozymes. In some embodiments, an H3K27 inhibitor is an EZH2 inhibitor.

As used herein, the terms “EZH2 inhibitor” and “EZH2 methyltransferase inhibitor” refer to any EZH2 inhibitor that is currently known in the art or that will be identified in the future, and includes any chemical entity that, upon administration to a patient, results in inhibition of a biological activity associated with EZH2 activity in the patient, including any of the downstream biological effects otherwise resulting from the binding of EZH2 to its natural ligand. Such EZH2 inhibitors include any agent that can block EZH2 methyltransferase or any of the downstream biological effects of EZH2 methyltransferase that are relevant to treating cancer in a patient. Such an inhibitor can act by binding directly to EZH2 and inhibiting its methyltransferase activity. Alternatively, such an inhibitor can act by occupying a non-EZH2 domain of the polycomb repressive complex 2 (PRC2), thereby making EZH2 inaccessible to chromatin so that its normal biological activity is prevented or reduced. Alternatively, such an inhibitor can act by modulating the association of PRC2 proteins, or enhance ubiquitination and endocytotic degradation of EZH2. EZH2 inhibitors include but are not limited to low molecular weight inhibitors, antibodies or antibody fragments, antisense constructs, small inhibitory RNAs (i.e., RNA interference by dsRNA; RNAi), and ribozymes. In one embodiment, the EZH2 inhibitor is a small organic molecule that binds specifically to the human EZH2, such as EPZ-6438, CPI-169, CPI-1205, EPZ005687, GSK-126, GSK343, and GSK503.

A “promoter,” as used herein, includes all sequences capable of driving transcription of a coding sequence in a cultured cell, e.g., a mammalian cell. Thus, promoters used in the methods of the invention include cis-acting transcriptional control elements and regulatory sequences that are involved in regulating or modulating the timing and/or rate of transcription of a gene (e.g., SMARCA2). For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) transcription.

A “patient” or “subject” herein refers to an animal (including, e.g., a mammal, such as a dog, a cat, a horse, a rabbit, a zoo animal, a cow, a pig, a sheep, a non-human primate, and a human) eligible for treatment who is experiencing, has experienced, has risk of developing, or has a family history of one or more signs, symptoms, or other indicators of a cell proliferative disease or disorder, such as a cancer. Intended to be included as a patient is any patient involved in clinical research trials not showing any clinical sign of disease, involved in epidemiological studies, or once used as controls. The patient may have been previously treated with an inhibitor of H3K27 methylation, another drug, or not previously treated. The patient may be naive to an additional drug(s) being used when the treatment is started, i.e., the patient may not have been previously treated with, for example, a therapy other than one including an inhibitor of H3K27 methylation (e.g., an EZH2 inhibitor) at “baseline” (i.e., at a set point in time before the administration of a first dose of an inhibitor of H3K27 methylation in the treatment method herein, such as the day of screening the subject before treatment is commenced). Such a “naive” patient or subject is generally considered a candidate for treatment with such additional drug(s).

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

“Polynucleotide” or “nucleic acid,” as used interchangeably herein, refers to polymers of nucleotides of any length and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro-, or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. A polynucleotide can contain one or more different types of modifications as described herein and/or multiple modifications of the same type. The preceding description applies to all polynucleotides referred to herein, including RNA and DNA.

“Oligonucleotide,” as used herein, generally refers to short, single stranded, polynucleotides that are, but not necessarily, less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “primer” refers to a single-stranded polynucleotide that is capable of hybridizing to a nucleic acid and allowing polymerization of a complementary nucleic acid, generally by providing a free 3′-OH group.

The term “small molecule” refers to any molecule with a molecular weight of about 2000 daltons or less (e.g., about 1500 daltons or less, or about 1000 daltons or less), preferably of about 750 daltons or less (e.g., between about 450-650 daltons, e.g., between about 500-600 daltons, e.g., between about 525-575 daltons).

The term “detection” includes any means of detecting, including direct and indirect detection.

The term “biomarker” as used herein refers to an indicator molecule or set of molecules (e.g., predictive, diagnostic, and/or prognostic indicator), which can be detected in a sample and includes, for example, a methylated histone (e.g., H3K27me3, e.g., an occupancy level of H3K27), SWI/SNF, or a SWI/SNF complex member or subunit (e.g., SMARCA2, e.g., an expression level of SMARCA2). The biomarker may be a predictive biomarker and serve as an indicator of the likelihood of sensitivity or benefit of a patient having a particular disease or disorder (e.g., a proliferative cell disorder (e.g., cancer)) to treatment with an inhibitor of H3K27 methylation. Biomarkers include, but are not limited to, polynucleotides (e.g., DNA and/or RNA (e.g., mRNA)), polynucleotide copy number alterations (e.g., DNA copy numbers), polypeptides, polypeptide and polynucleotide modifications (e.g., post-translational modifications), carbohydrates, and/or glycolipid-based molecular markers. In some embodiments, a biomarker is a gene.

The “amount” or “level” of a biomarker, as used herein, is a detectable level in a biological sample. These can be measured by methods known to one skilled in the art and also disclosed herein.

The term “level of expression” or “expression level” generally refers to the amount of a biomarker in a biological sample. “Expression” generally refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

The term “occupancy level,” as used herein, refers to the degree of methylation (e.g., monomethylation or, preferably, di-, or trimethylation of a histone (e.g., histone H3) at one or more histone methylation sites (e.g., lysine 27 of histone H3 (H3K27)). Occupancy level at a specific genomic region can be assessed by chromatin immunoprecipitation (ChIP) techniques, such as ChIP-seq or ChIP-PCR.

“Increased expression,” “increased expression level,” “increased levels,” “elevated expression,” “elevated expression levels,” or “elevated levels” refers to an increased expression or increased levels of a biomarker in an individual relative to a control, such as an individual or individuals who do not have the disease or disorder (e.g., cancer), an internal control (e.g., a housekeeping biomarker), or a median expression level of the biomarker in samples from a group/population of patients.

“Decreased expression,” “decreased expression level,” “decreased levels,” “reduced expression,” “reduced expression levels,” or “reduced levels” refers to a decrease expression or decreased levels of a biomarker in an individual relative to a control, such as an individual or individuals who do not have the disease or disorder (e.g., cancer), an internal control (e.g., a housekeeping biomarker), or a median expression level of the biomarker in samples from a group/population of patients. In some embodiments, reduced expression is little or no expression.

The term “housekeeping gene” refers herein to a gene or group of genes that encode proteins whose activities are essential for the maintenance of cell function and which are typically similarly present in all cell types.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology, (Stockton Press, N Y, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

“Quantitative real-time polymerase chain reaction” or “qRT-PCR” refers to a form of PCR wherein the amount of PCR product is measured at each step in a PCR reaction. This technique has been described in various publications including, for example, Cronin et al., Am. J. Pathol. 164(1):35-42 (2004) and Ma et al., Cancer Cell 5:607-616 (2004).

The term “microarray” refers to an ordered arrangement of hybridizable array elements, preferably polynucleotide probes, on a substrate.

The term “sample,” as used herein, refers to a composition that is obtained or derived from a subject (e.g., individual of interest) that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples (e.g., tumor tissue samples), primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. For instance, a “tumor sample” is a tissue sample obtained from a tumor or other cancerous tissue. The tissue sample may contain a mixed population of cell types (e.g., tumor cells and non-tumor cells, cancerous cells and non-cancerous cells). The tissue sample may contain compounds which are not naturally intermixed with the tissue in nature such as preservatives, anticoagulants, buffers, fixatives, nutrients, antibiotics, or the like.

A “reference sample,” “reference cell,” “reference tissue,” “control sample,” “control cell,” or “control tissue,” as used herein, refers to a sample, cell, tissue, standard, or level that is used for comparison purposes. In one embodiment, a reference level, reference sample, reference cell, reference tissue, control sample, control cell, or control tissue is obtained from a healthy and/or non-diseased part of the body (e.g., tissue or cells) of the same subject or individual. For example, the reference level, reference sample, reference cell, reference tissue, control sample, control cell, or control tissue may be healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference level, reference sample, reference cell, reference tissue, control sample, control cell, or control tissue is obtained from a healthy and/or non-diseased part of the body (e.g., tissues or cells) of an individual who is not the subject or individual. In even another embodiment, a reference level, reference sample, reference cell, reference tissue, control sample, control cell, or control tissue is obtained from an untreated tissue and/or cell of the body of an individual who is not the subject or individual.

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

“Individual response” or “response” can be assessed using any endpoint indicating a benefit to the individual, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues; (4) inhibition (i.e., reduction, slowing down, or complete stopping) of metastasis; (5) relief, to some extent, of one or more symptoms associated with the disease or disorder (e.g., cancer); (6) increase or extension in the length of survival, including overall survival and progression free survival; and/or (7) decreased mortality at a given point of time following treatment.

An “effective response” of a patient or a patient's “responsiveness” to treatment with a medicament and similar wording refers to the clinical or therapeutic benefit imparted to a patient at risk for, or having a, a disease or disorder, such as cancer. In one embodiment, such benefit includes any one or more of: extending survival (including overall survival and/or progression-free survival); resulting in an objective response (including a complete response or a partial response); or improving signs or symptoms of cancer. In one embodiment, at least one biomarker (e.g., the expression level of SMARCA2 or the occupancy level of H3K27 (e.g., H3K27me3) at a SMARCA2 promoter) is used to identify a patient who is predicted to have an increased likelihood of being responsive to treatment with a medicament (e.g., treatment comprising an inhibitor of H3K27 methylation), relative to a patient who does not express the at least one biomarker. In one embodiment, the at least one biomarker (e.g., the expression level of SMARCA2 or the occupancy level of H3K27 (e.g., H3K27me3) at a SMARCA2 promoter) is used to identify the patient who is predicted to have an increase likelihood of being responsive to treatment with a medicament (e.g., an inhibitor of H3K27 methylation), relative to a patient who does not express the at least one biomarker at the same level.

A “therapeutically effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), response rates (e.g., CR and PR), duration of response, and/or quality of life.

A “disorder” is any condition that would benefit from treatment including, but not limited to, chronic and acute disorders or diseases including those pathological conditions which predispose the mammal to the disorder in question.

A “mutation” is a deletion, insertion, or substitution of a nucleotide(s) relative to a reference nucleotide sequence, such as a wildtype sequence.

The phrase “identifying a mutation” refers to the act of comparing a nucleotide sequence in a sample with a reference nucleotide sequence, such as a wildtype nucleotide sequence, to identify a deletion, insertion, or substitution in the sequence.

The terms “cancer” and “cancerous” refer to or describe the physiological condition in mammals that is typically characterized by unregulated cell growth. Included in this definition are benign and malignant cancers. Examples of cancer include, but are not limited to, rhabdoid cancer; carcinoma; lymphoma; blastoma (including medulloblastoma and retinoblastoma); sarcoma (including liposarcoma and synovial cell sarcoma); neuroendocrine tumors (including carcinoid tumors, gastrinoma, and islet cell cancer); mesothelioma; schwannoma (including acoustic neuroma); meningioma; adenocarcinoma; melanoma; and leukemia or lymphoid malignancies. More particular examples of such cancers include ovarian cancer (e.g., ovarian clear cell carcinoma, or small cell carcinoma of the ovary, e.g., a small cell carcinoma of the ovary, hypercalcemic type), bladder cancer (e.g., urothelial bladder cancer (e.g., transitional cell or urothelial carcinoma, non-muscle invasive bladder cancer, muscle-invasive bladder cancer, and metastatic bladder cancer) and non-urothelial bladder cancer); squamous cell cancer (e.g., epithelial squamous cell cancer); lung cancer, including small-cell lung cancer (SCLC), non-small cell lung cancer (NSCLC), adenocarcinoma of the lung, and squamous carcinoma of the lung; cancer of the peritoneum; hepatocellular cancer; gastric or stomach cancer, including gastrointestinal cancer; pancreatic cancer; glioblastoma; cervical cancer; ovarian cancer; liver cancer; hepatoma; breast cancer (including metastatic breast cancer); colon cancer; rectal cancer; colorectal cancer; endometrial or uterine carcinoma; salivary gland carcinoma; kidney or renal cancer; prostate cancer; vulval cancer; thyroid cancer; hepatic carcinoma; anal carcinoma; penile carcinoma; Merkel cell cancer; mycoses fungoids; testicular cancer; esophageal cancer; tumors of the biliary tract; head and neck cancer; and hematological malignancies. In some embodiments, the cancer is rhabdoid cancer (e.g., malignant rhabdoid cancer, teratoid/rhabdoid cancer, pediatric rhabdoid cancer). In some embodiments, the cancer is a rhabdoid cancer of the kidney (e.g., a renal cancer or adrenal cancer), brain, or other soft tissues. In some embodiments, the ovarian cancer is a small cell carcinoma of the ovary, e.g., a small cell carcinoma of the ovary, hypercalcemic type. Any cancer can be at early stage or at late stage. By “early stage cancer” or “early stage tumor” is meant a cancer that is not invasive or metastatic or is classified as a Stage 0, 1, or 2 cancer.

The term “tumor,” as used herein, refers to all neoplastic cell growth and proliferation, whether malignant or benign, and all pre-cancerous and cancerous cells and tissues. The terms “cancer,” “cancerous,” and “tumor” are not mutually exclusive as referred to herein.

A “SUZ12 antagonist” refers to a molecule capable of binding to a SUZ12, reducing SUZ12 expression levels, or neutralizing, blocking, inhibiting, abrogating, reducing, or interfering with SUZ12 biological activities, including, but not limited to, SUZ12 signaling and SUZ12-mediated methyltransferase activity. For example, a molecule capable of neutralizing, blocking, inhibiting, abrogating, reducing, or interfering with SUZ12 biological activities can exert its effects by binding to one or more SUZ12 binding sites on a PRC2 protein (e.g., EED or jumonji). Included as SUZ12-specific antagonists useful in the methods of the invention are polypeptides that specifically bind to SUZ12, anti-SUZ12 antibodies, and antigen-binding fragments thereof. SUZ12-specific antagonists also include antagonist variants of SUZ12 polypeptides, antisense nucleobase oligomers complementary to at least a fragment of a nucleic acid molecule encoding a SUZ12 polypeptide; small RNAs complementary to at least a fragment of a nucleic acid molecule encoding a SUZ12 polypeptide; ribozymes that target SUZ12; peptibodies to SUZ12; and SUZ12 aptamers. SUZ12-specific antagonists also include nonpeptide small molecules that bind to SUZ12 and are capable of blocking, inhibiting, abrogating, reducing, or interfering with SUZ12 biological activities. In certain embodiments, the SUZ12 antagonist reduces or inhibits, by at least 10%, 20%, 30%, 40%, 50%, 60%, 70%, 80%, 90% or more, the expression level or biological activity of SUZ12.

An “EED antagonist” refers to a molecule capable of binding to a EED, reducing EED expression levels, or neutralizing, blocking, inhibiting, abrogating, reducing, or interfering with EED biological activities, including, but not limited to, EED signaling and EED-mediated methyltransferase activity. For example, a molecule capable of neutralizing, blocking, inhibiting, abrogating, reducing, or interfering with EED biological activities can exert its effects by binding to one or more EED binding sites on a PRC2 protein (e.g., SUZ12 or jumonji). Included as EED-specific antagonists useful in the methods of the invention are polypeptides that specifically bind to EED, anti-EED antibodies, and antigen-binding fragments thereof. EED-specific antagonists also include antagonist variants of EED polypeptides, antisense nucleobase oligomers complementary to at least a fragment of a nucleic acid molecule encoding a EED polypeptide; small RNAs complementary to at least a fragment of a nucleic acid molecule encoding a EED polypeptide; ribozymes that target EED; peptibodies to EED; and EED aptamers. EED-specific antagonists also include nonpeptide small molecules that bind to EED and are capable of blocking, inhibiting, abrogating, reducing, or interfering with EED biological activities. In certain embodiments, the EED antagonist reduces or inhibits, by at least 10%, 20%, 30%, 40%, 50%, 60%, 70%, 80%, 90% or more, the expression level or biological activity of EED.

A “jumonji antagonist” refers to a molecule capable of binding to a jumonji, reducing jumonji expression levels, or neutralizing, blocking, inhibiting, abrogating, reducing, or interfering with jumonji biological activities, including, but not limited to, jumonji signaling and jumonji-mediated methyltransferase activity. For example, a molecule capable of neutralizing, blocking, inhibiting, abrogating, reducing, or interfering with jumonji biological activities can exert its effects by binding to one or more jumonji binding sites on a PRC2 protein (e.g., SUZ12 or EED). Included as jumonji-specific antagonists useful in the methods of the invention are polypeptides that specifically bind to jumonji, anti-jumonji antibodies, and antigen-binding fragments thereof. Jumonji-specific antagonists also include antagonist variants of jumonji polypeptides, antisense nucleobase oligomers complementary to at least a fragment of a nucleic acid molecule encoding a jumonji polypeptide; small RNAs complementary to at least a fragment of a nucleic acid molecule encoding a jumonji polypeptide; ribozymes that target jumonji; peptibodies to jumonji; and jumonji aptamers. Jumonji-specific antagonists also include nonpeptide small molecules that bind to jumonji and are capable of blocking, inhibiting, abrogating, reducing, or interfering with jumonji biological activities. In certain embodiments, the jumonji antagonist reduces or inhibits, by at least 10%, 20%, 30%, 40%, 50%, 60%, 70%, 80%, 90% or more, the expression level or biological activity of jumonji.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable excipient” refers to an ingredient in a pharmaceutical formulation, other than an active ingredient, which is nontoxic to a subject. A pharmaceutically acceptable excipient includes, but is not limited to, a buffer, carrier, stabilizer, or preservative.

The term “pharmaceutically acceptable salt” denotes salts which are not biologically or otherwise undesirable. Pharmaceutically acceptable salts include both acid and base addition salts. The phrase “pharmaceutically acceptable” indicates that the substance or composition must be compatible chemically and/or toxicologically, with the other ingredients comprising a formulation, and/or the mammal being treated therewith.

The term “pharmaceutically acceptable acid addition salt” denotes those pharmaceutically acceptable salts formed with inorganic acids, such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids, such as formic acid, acetic acid, propionic acid, glycolic acid, gluconic acid, lactic acid, pyruvic acid, oxalic acid, malic acid, maleic acid, malonic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid “mesylate”, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines, including naturally occurring substituted amines, cyclic amines, and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, and polyamine resins.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, inhibitors of H3K27 methylation (e.g., an EZH2 inhibitor) are used to delay development of a disease or to slow the progression of a disease.

The term “anti-cancer therapy” refers to a therapy useful in treating cancer. Examples of anti-cancer therapeutic agents include, but are limited to, cytotoxic agents, chemotherapeutic agents, growth inhibitory agents, agents used in radiation therapy, anti-angiogenesis agents, apoptotic agents, anti-tubulin agents, and other agents to treat cancer, for example, anti-CD20 antibodies, platelet derived growth factor inhibitors (e.g., GLEEVEC™ (imatinib mesylate)), a COX-2 inhibitor (e.g., celecoxib), interferons, cytokines, antagonists (e.g., neutralizing antibodies) that bind to one or more of the following targets PDGFR-β, BlyS, APRIL, BCMA receptor(s), TRAIL/Apo2, other bioactive and organic chemical agents, and the like. Combinations thereof are also included in the invention.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³², and radioactive isotopes of Lu), chemotherapeutic agents, e.g., methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (see, e.g., Nicolaou et al., Angew. Chem Intl. Ed. Engl., 33: 183-186 (1994)); dynemicin, including dynemicin A; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycin, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, for example taxanes including TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® docetaxel (Rhône-Poulenc Rorer, Antony, France); chloranbucil; gemcitabine (GEMZAR®); 6-thioguanine; mercaptopurine; methotrexate; platinum or platinum-based chemotherapy agents and platinum analogs, such as cisplatin, carboplatin, oxaliplatin (ELOXATIN™), satraplatin, picoplatin, nedaplatin, triplatin, and lipoplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylornithine (DMFO); retinoids such as retinoic acid; capecitabine (XELODA®); pharmaceutically acceptable salts or acids of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovorin. Additional chemotherapeutic agents include the cytotoxic agents useful as antibody drug conjugates, such as maytansinoids (DM1, for example) and the auristatins MMAE and MMAF, for example.

Chemotherapeutic agents also include “anti-hormonal agents” or “endocrine therapeutics” that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), EVISTA® raloxifene, droloxifene, 4-hydroxytamoxifen, trioxifene, keoxifene, LY117018, onapristone, and FARESTON® toremifene; anti-progesterones; estrogen receptor down-regulators (ERDs); agents that function to suppress or shut down the ovaries, for example, leutinizing hormone-releasing hormone (LHRH) agonists such as LUPRON® and ELIGARD® leuprolide acetate, goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole. In addition, such definition of chemotherapeutic agents includes bisphosphonates such as clodronate (for example, BONEFOS® or OSTAC®), DIDROCAL® etidronate, NE-58095, ZOMETA® zoledronic acid/zoledronate, FOSAMAX® alendronate, AREDIA® pamidronate, SKELID® tiludronate, or ACTONEL® risedronate; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGFR); vaccines such as THERATOPE® vaccine and gene therapy vaccines, for example, ALLOVECTIN® vaccine, LEUVECTIN® vaccine, and VAXID® vaccine; LURTOTECAN® topoisomerase 1 inhibitor; ABARELIX® rmRH; lapatinib ditosylate (an ErbB-2 and EGFR dual tyrosine kinase small-molecule inhibitor also known as GW572016); and pharmaceutically acceptable salts or acids of any of the above.

Chemotherapeutic agents also include antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories), which is a recombinant exclusively human-sequence, full-length IgG1 λ antibody genetically modified to recognize interleukin-12 p40 protein.

Chemotherapeutic agents also include “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6.3, and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP 659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO 98/14451, WO 98/50038, WO 99/09016, and WO 99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); and dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitors such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035, 4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca). Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (ENBREL®), infliximab (REMICADE®), adalimumab (HUMIRA®), certolizumab pegol (CIMZIA®), golimumab (SIMPONI®), Interleukin 1 (IL-1) blockers such as anakinra (KINERET®), T-cell co-stimulation blockers such as abatacept (ORENCIA®), Interleukin 6 (IL-6) blockers such as tocilizumab (ACTEMERA®); Interleukin 13 (IL-13) blockers such as lebrikizumab; Interferon alpha (IFN) blockers such as rontalizumab; beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); miscellaneous investigational agents such as thioplatin, PS-341, phenylbutyrate, ET-18-OCH3, and farnesyl transferase inhibitors (L-739749, L-744832); polyphenols such as quercetin, resveratrol, piceatannol, epigallocatechine gallate, theaflavins, flavanols, procyanidins, betulinic acid; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g., celecoxib or etoricoxib), proteosome inhibitor (e.g., PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts or acids of any of the above; as well as combinations of two or more of the above.

The term “prodrug” as used herein refers to a precursor form of a pharmaceutically active substance that is less cytotoxic to tumor cells compared to the parent drug and is capable of being enzymatically activated or converted into the more active parent form. See, for example, Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

A “growth inhibitory agent” when used herein refers to a compound or composition which inhibits growth and/or proliferation of a cell (e.g., a cell whose growth is dependent on H3K27me3) either in vitro or in vivo. Thus, the growth inhibitory agent may be one that significantly reduces the percentage of cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as the anthracycline antibiotic doxorubicin ((8S-cis)-10-[(3-amino-2,3,6-trideoxy-α-L-Iyxo-hexapyranosyl)oxy]-7,8,9,10-tetrahydro-6,8,11-trihydroxy-8-(hydroxyacetyl)-1-methoxy-5,12-naphthacenedione), epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in “The Molecular Basis of Cancer,” Mendelsohn and Israel, eds., Chapter 1, entitled “Cell cycle regulation, oncogenes, and antineoplastic drugs” by Murakami et al. (WB Saunders: Philadelphia, 1995), especially p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one-time administration and typical dosages range from 10 to 200 units (Grays) per day.

As used herein, “administering” is meant a method of giving a dosage of a compound (e.g., an inhibitor or antagonist) or a pharmaceutical composition (e.g., a pharmaceutical composition including an inhibitor or antagonist) to a subject (e.g., a patient). Administering can be by any suitable means, including parenteral, intrapulmonary, and intranasal, and, if desired for local treatment, intralesional administration. Parenteral infusions include, for example, intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The term “co-administered” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

By “reduce or inhibit” is meant the ability to cause an overall decrease of 20%, 30%, 40%, 50%, 60%, 70%, 75%, 80%, 85%, 90%, 95%, or greater. Reduce or inhibit can refer, for example, to the level of activity and/or function of, e.g., EZH2 or an agonist of EZH2. Additionally, Reduce or inhibit can refer, for example, to the symptoms of the disorder being treated, the presence or size of metastases, or the size of the primary tumor.

The term “package insert” is used to refer to instructions customarily included in commercial packages of therapeutic products, that contain information about the indications, usage, dosage, administration, combination therapy, contraindications, and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a probe for specifically detecting a biomarker (e.g., an expression level of SMARCA2 or an occupancy level of H3K27 (e.g., H3K27me3) at a SMARCA2 promoter) described herein. In certain embodiments, the manufacture or kit is promoted, distributed, or sold as a unit for performing the methods described herein.

The phrase “based on” when used herein means that the information about one or more biomarkers is used to inform a diagnostic decision, a treatment decision, information provided on a package insert, or marketing/promotional guidance, etc.

### III. Methods

A. Diagnostic Methods

The present invention provides methods for identifying and/or monitoring patients having cancer (e.g., rhabdoid cancer (e.g., malignant rhabdoid cancer, e.g., malignant rhabdoid brain cancer or malignant rhabdoid renal cancer)), ovarian cancer (e.g., ovarian clear cell carcinoma, or a small cell carcinoma of the ovary, e.g., a small cell carcinoma of the ovary, hypercalcemic type), lung cancer, gastric cancer, bladder cancer, breast cancer, skin cancer, colorectal cancer, stomach cancer, lymphoid cancer, cervical cancer, peritoneal cancer, pancreatic cancer, glioblastoma, liver cancer, bladder cancer, colon cancer, endometrial cancer, uterine cancer, renal cancer, prostate cancer, thyroid cancer, and head and neck cancer)) who may benefit from treatment including one or more inhibitors of histone 3 lysine 27 (H3K27) methylation (e.g., H3K27me3). The methods include detecting one or more biomarkers in a sample (e.g., a tissue sample (e.g., a tumor tissue sample)) from a patient, wherein one or more such biomarkers is indicative of whether the patient is sensitive or responsive to a treatment including one or more inhibitors of H3K27 methylation, such as an inhibitor of H3K27 methylation, e.g., an EZH2 inhibitor, e.g., EPZ-6438. Also provided are methods for optimizing therapeutic efficacy for treatment of a patient having a cancer, wherein the treatment includes one or more inhibitors of H3K27 methylation. Further provided herein are methods for predicting responsiveness of a patient having a cancer to treatment including one or more inhibitors of H3K27 methylation. Also, provided herein are methods for selecting a therapy for a patient having a cancer. Any of the methods may further include administering to the patient a therapeutically effective amount of an inhibitor of H3K27 methylation to the patient. In addition, any of the methods may further include administering an effective amount of an additional therapeutic agent (e.g., a second therapeutic agent, e.g., a second inhibitor of H3K27 methylation or an anti-cancer agent) to the patient.

The invention provides methods for identifying a patient having a cancer who may benefit from treatment including one or more inhibitors of H3K27 methylation, optimizing therapeutic efficacy for treatment of a patient having cancer, predicting responsiveness of a patient having a cancer to treatment including one or more inhibitors of H3K27 methylation, and selecting a therapy for a patient having a cancer, based on determining an expression level of SMARCA2 in a sample obtained from the patient, wherein an decreased expression level of the SMARCA2 in the sample as compared to a reference level indicates that the patient has an increased likelihood of benefiting from treatment including one or more inhibitors of H3K27 methylation. More particularly, any of the preceding methods may be based on determining the expression level of SMARCA2 in a sample from a patient useful for monitoring whether the patient is responsive or sensitive to inhibition of H3K27 methylation (e.g., inhibition of H3K27me3).

The invention further provides methods for identifying a patient having a cancer who may benefit from treatment including one or more inhibitors of H3K27 methylation, optimizing therapeutic efficacy for treatment of a patient having cancer, predicting responsiveness of a patient having a cancer to treatment including one or more inhibitors of H3K27 methylation, and selecting a therapy for a patient having a cancer, based on determining an occupancy level of H3K27 (e.g., H3K27me3) at a SMARCA2 promoter in a sample obtained from the patient, wherein an increased occupancy level of H3K27 (e.g., as measured by detection of mono-, di-, or trimethylation at H3K27 (H3K27me3)) at the SMARCA2 promoter in the sample as compared to a reference occupancy level indicates that the patient has an increased likelihood of benefiting from treatment including one or more inhibitors of H3K27 methylation. More particularly, any of the preceding methods may be based on determining the occupancy level of H3K27 (e.g., H3K27me3) at a SMARCA2 promoter in a sample from a patient useful for monitoring whether the patient is responsive or sensitive to inhibition of H3K27 methylation (e.g., inhibition of H3K27me3).

The disclosed methods and assays provide for convenient, efficient, and potentially cost-effective means to obtain data and information useful in assessing appropriate or effective therapies for treating patients. For example, a patient can provide a tissue sample (e.g., a tumor biopsy or a blood sample) before and/or after treatment with an inhibitor of H3K27 methylation and the sample can be examined by way of various in vitro assays to determine whether the patient's cells are sensitive to inhibition of H3K27 methylation, e.g., by an inhibitor of H3K27 methylation, such as an EZH2 inhibitor (e.g., EPZ-6438).

The invention also provides methods for monitoring the sensitivity or responsiveness of a patient to an inhibitor of H3K27 methylation. The methods may be conducted in a variety of assay formats, including assays detecting genetic or protein expression levels, biochemical assays detecting appropriate activity, and/or immunoassays (e.g., immunoprecipitation, e.g., chromatin immunoprecipitation (ChIP) assay).

Determination of an expression level of SMARCA2 in patient samples can be predictive of whether a patient is sensitive to one or more of the biological effects of an inhibitor of H3K27 methylation. A lower expression level (i.e., repression) of SMARCA2 in a sample from a patient having a cancer relative to a reference level correlates with treatment efficacy of such a patient with an inhibitor of H3K27 methylation. A reference expression level can be the expression level of SMARCA2 in a sample from a group/population of patients being tested for responsiveness to an inhibitor of H3K27 methylation or the mean or median expression level of SMARCA2 in a sample from a group/population of patients having a particular cancer, e.g., a cancer not associated with a mutation in a SWI/SNF complex protein, or a sample from a healthy or noncancerous tissue.

Similarly, determination of H3K27 (e.g., H3K27me3) occupancy levels at a SMARCA2 promoter in a sample obtained from a patient can be predictive of whether a patient is or will be sensitive to the biological effects of an inhibitor of H3K27 methylation. An increased occupancy level of H3K27 (e.g., H3K27me3) at a SMARCA2 promoter in a sample from a patient having a cancer relative to a reference level correlates with treatment efficacy of such a patient with an inhibitor of H3K27 methylation. A reference occupancy level can be the occupancy level of H3K27 (e.g., H3K27me3) at a SMARCA2 promoter in a sample from a group/population of patients being tested for responsiveness to an inhibitor of H3K27 methylation or the mean or median occupancy level of H3K27 (e.g., H3K27me3) at a SMARCA2 promoter in a sample from a group/population of patients having a particular cancer, e.g., a cancer not associated with a mutation in a SWI/SNF complex protein, or a sample from a healthy or noncancerous tissue.

Assessment of either SMARCA2 expression or H3K27 occupancy at a SMARCA2 promoter, or both, can also be used to monitor a patient's response to an inhibitor of H3K27 methylation (e.g., an H3K27me3 inhibitor, e.g., an EZH2 inhibitor). A patient who has been determined to be responsive to treatment with an inhibitor of H3K27 methylation can be monitored over the course of treatment by comparing biomarkers in samples obtained prior to beginning treatment (e.g., with one or more inhibitors of H3K27 methylation) with the corresponding biomarkers in samples obtained after treatment. In some cases, increasing SMARCA2 expression levels over the course of treatment with an inhibitor of H3K27 methylation indicates that a patient is responsive to the treatment. Similarly, according to some embodiments, decreasing occupancy of H3K27 (e.g., H3K27me3) at a SMARCA2 promoter in a sample over the course of treatment with an inhibitor of H3K27 methylation indicates that a patient is responding to the treatment.

In one aspect, the invention provides a method of determining whether a patient having a cancer will respond to treatment with an inhibitor of H3K27 methylation including determining the expression level of SMARCA2 in a sample from the patient obtained (i) before an inhibitor of H3K27 methylation has been administered to the patient, (ii) after an inhibitor of H3K27 methylation has been administered to the patient, or (iii) before and after such treatment. A change (e.g., decrease) in the expression of SMARCA2 relative to a reference expression level indicates that the patient will likely respond to treatment with an inhibitor of H3K27 methylation. In some embodiments, the patient may be informed that they have an increased likelihood of responding to treatment with an inhibitor of H3K27 methylation and/or provided a recommendation that an anti-cancer therapy include one or more inhibitors of H3K27 methylation.

In another aspect, the invention provides a method of optimizing therapeutic efficacy of an anti-cancer therapy for a patient, including detecting, as a biomarker, an expression level of SMARCA2 in a sample from the patient obtained (i) before an inhibitor of H3K27 methylation has been administered to the patient, (ii) after any inhibitor of H3K27 methylation has been administered to the patient, or (iii) before and after such treatment. In some cases, a change (e.g., decrease) in the expression of SMARCA2 relative to a reference level indicates that the patient will likely respond to treatment with an inhibitor of H3K27 methylation. The patient may be informed that they have an increased likelihood of responding to treatment with an inhibitor of H3K27 methylation and/or provided a recommendation that anti-cancer therapy include an inhibitor of H3K27 methylation.

In another aspect, the invention provides a method for selecting a therapy for a patient having a cancer, including detecting, as a biomarker, the expression of SMARCA2 in a sample from the patient obtained (i) before any inhibitor of H3K27 methylation has been administered to the patient, (ii) after any inhibitor of H3K27 methylation has been administered to the patient, or (iii) before and after such treatment. In some cases, a change (e.g., decrease) in the expression of the SMARCA2 relative to a reference level indicates that the patient will likely respond to treatment with an inhibitor of H3K27 methylation. The patient may be informed that they have an increased likelihood of responding to treatment with an inhibitor of H3K27 methylation and/or provided a recommendation that an anti-cancer therapy include an inhibitor of H3K27 methylation.

In another embodiment, the present invention provides a method of monitoring the sensitivity or responsiveness of a patient to an inhibitor of H3K27 methylation. This method includes assessing an expression level of SMARCA2 in a patient sample and predicting the sensitivity or responsiveness of the patient to the inhibitor of H3K27 methylation, wherein a change (e.g., an increase or a decrease) in the expression of SMARCA2 correlates with sensitivity or responsiveness of the patient to effective treatment with the inhibitor of H3K27 methylation.

According to one embodiment of this method, a biological sample is obtained from the patient before administration of an inhibitor of H3K27 methylation and subjected to an assay to evaluate the level of expression products of SMARCA2 in the sample. If expression of SMARCA2 is decreased relative to a reference expression level, the patient is determined to be sensitive or responsive to treatment with an inhibitor of H3K27 methylation. The patient may be informed that they have an increased likelihood of being sensitive or responsive to treatment with an inhibitor of H3K27 methylation and/or provided a recommendation that anti-cancer therapy include an inhibitor of H3K27 methylation. In another embodiment of this method, a biological sample is obtained from the patient before and after administration of an inhibitor of H3K27 methylation and subjected to an assay to evaluate the level of expression products of SMARCA2 in the sample. If expression of SMARCA2 is increased after administration of an inhibitor of H3K27 methylation relative to the sample obtained prior to administration of the inhibitor of H3K27 methylation, the patient is determined to be responsive to the treatment, and the patient may be advised to continue treatment with the inhibitor of H3K27 methylation.

In a separate aspect, the invention provides a method of determining whether a patient having a cancer will respond to treatment with an inhibitor of H3K27 methylation including determining the occupancy level of H3K27 (e.g., H3K27me3) at a SMARCA2 promoter in a sample from the patient obtained (i) before any inhibitor of H3K27 methylation has been administered to the patient, (ii) after an inhibitor of H3K27 methylation has been administered to the patient, or (iii) before and after such treatment. In some embodiments, a change (e.g., decrease) in the occupancy of H3K27 (e.g., H3K27me3) at the SMARCA2 promoter relative to a reference level indicates that the patient will likely respond to treatment with an inhibitor of H3K27 methylation. The patient may be informed that they have an increased likelihood of responding to treatment with an inhibitor of H3K27 methylation and/or provided a recommendation that anti-cancer therapy include an inhibitor of H3K27 methylation.

In another aspect, the invention provides a method of optimizing therapeutic efficacy of an anti-cancer therapy for a patient, including detecting, as a biomarker, occupancy of H3K27 (e.g., H3K27me3) at a SMARCA2 promoter in a sample from the patient obtained (i) before any inhibitor of H3K27 methylation has been administered to the patient, (ii) after an inhibitor of H3K27 methylation has been administered to the patient, or (iii) before and after such treatment. In some cases, a change (e.g., decrease) in the occupancy level of H3K27 (e.g., H3K27me3) at a SMARCA2 promoter relative to a reference level indicates that the patient will likely respond to treatment with an inhibitor of H3K27 methylation. The patient may be informed that they have an increased likelihood of responding to treatment with an inhibitor of H3K27 methylation and/or provided a recommendation that anti-cancer therapy include an inhibitor of H3K27 methylation.

In another aspect, the invention provides a method for selecting a therapy for a patient having a cancer, including detecting, as a biomarker, the occupancy of H3K27 (e.g., H3K27me3) at a SMARCA2 promoter in a sample from the patient obtained (i) before any inhibitor of H3K27 methylation has been administered to the patient, (ii) after an inhibitor of H3K27 methylation has been administered to the patient, or (iii) before and after such treatment. In some cases, a change (e.g., decrease) in the occupancy level of H3K27 (e.g., H3K27me3) at a SMARCA2 promoter relative to a reference level indicates that the patient will likely respond to treatment with an inhibitor of H3K27 methylation. The patient may be informed that they have an increased likelihood of responding to treatment with an inhibitor of H3K27 methylation and/or provided a recommendation that anti-cancer therapy include an inhibitor of H3K27 methylation.

In another embodiment, the present invention provides a method of monitoring the sensitivity or responsiveness of a patient to an inhibitor of H3K27 methylation. This method including assessing the occupancy level of H3K27 (e.g., H3K27me3) at a SMARCA2 promoter in a patient sample and predicting the sensitivity or responsiveness of the patient to one or more inhibitors of H3K27 methylation, wherein a change (e.g., an increase or a decrease) in the expression of SMARCA2 correlates with sensitivity or responsiveness of the patient to effective treatment with the one or more inhibitors of H3K27 methylation.

According to one embodiment of this method, a biological sample is obtained from the patient before administration of any inhibitor of H3K27 methylation and subjected to an assay to evaluate the level of occupancy of H3K27 (e.g., H3K27me3) at a SMARCA2 promoter in the sample. In some cases, if the occupancy level of H3K27 (e.g., H3K27me3) at a SMARCA2 promoter is increased relative to a reference occupancy level, the patient is determined to be sensitive or responsive to treatment with an inhibitor of H3K27 methylation. The patient may be informed that they have an increased likelihood of being sensitive or responsive to treatment with an inhibitor of H3K27 methylation and/or provided a recommendation that anti-cancer therapy include an inhibitor of H3K27 methylation. In another embodiment of this method, a biological sample is obtained from the patient before and after administration of an inhibitor of H3K27 methylation and subjected to an assay to evaluate the level of occupancy of H3K27 (e.g., H3K27me3) at a SMARCA2 promoter in the sample. If level of occupancy of H3K27 (e.g., H3K27me3) at the SMARCA2 promoter is increased after administration of an inhibitor of H3K27 methylation relative to the sample obtained prior to administration of the inhibitor of H3K27 methylation, the patient is determined to be responsive to the treatment, and the patient may be advised to continue treatment with the inhibitor of H3K27 methylation.

In some embodiments of any of the preceding methods, the expression level of SMARCA2 in a sample (e.g., a tissue sample (e.g., a tumor tissue sample)) obtained from the patient is determined to be decreased by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 98% or more, about 99% or more, or about 100%, e.g., from about 1% to about 5%, from about 5% to about 10%, from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, from about 90% to about 100%, from about 1% to about 10%, from about 10% to about 20%, from about 20% to about 30%, from about 30% to about 40%, from about 40% to about 50%, from about 50% to about 60%, from about 60% to about 70%, from about 70% to about 80%, from about 80% to about 90%, from about 90% to about 100%, from about 1% to about 25%, from about 25% to about 50%, from about 50% to about 75%, or from about 75% to about 100%) relative to a reference expression level.

In some embodiments of any of the methods, decreased expression level refers to an overall decrease as compared to a reference sample, reference cell, reference tissue, control sample, control cell, control tissue, or internal control (e.g., housekeeping gene).

Alternatively, the expression level of SMARCA2 in a sample (e.g., a tissue sample (e.g., a tumor tissue sample)) obtained from the patient can be determined to be increased (e.g., at a time point after the beginning of administration of treatment with an inhibitor of H3K27 methylation relative to a time point prior to the beginning of administration of treatment with an inhibitor of H3K27 methylation). In some embodiments, the expression level of SMARCA2 in a sample is increased by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 100% or more, about 110% or more, about 120% or more, about 130% or more, about 140% or more, about 150% or more, about 200% or more, about 250% or more, about 300% or more, about 350% or more, about 400% or more, about 450% or more, about 500% or more, about 550% or more, about 600% or more, about 650% or more, about 700% or more, about 750% or more, about 800% or more, about 850% or more, about 900% or more, about 950% or more, about 1,000% or more, about 2,000% or more, about 5,000% or more, or about 10,000% or more, e.g., from about 1% to about 5%, from about 5% to about 10%, from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, from about 95% to about 100%, from about 100% to about 200%, from about 200% to about 300%, from about 300% to about 400%, from about 400% to about 500%, from about 500% to about 600%, from about 600% to about 700%, from about 700% to about 800%, from about 800% to about 1,000%, from about 1,000% to about 2,000%, from about 2,000% to about 5,000%, from about 5,000% to about 10,000%, from about 1% to about 10%, from about 10% to about 20%, from about 20% to about 30%, from about 30% to about 40%, from about 40% to about 50%, from about 50% to about 60%, from about 60% to about 70%, from about 70% to about 80%, from about 80% to about 90%, from about 90% to about 100%, from about 1% to about 25%, from about 25% to about 50%, from about 50% to about 75%, from about 75% to about 100%, from about 1,000% to about 5,000%, or from about 5,000% to about 10,000%) relative to a reference expression level (e.g., at a time point prior to beginning treatment with an H3K27). In some embodiments, the expression level of SMARCA1 in a sample is increased (e.g., by about 1-fold, by about 1.1-fold, about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 3-fold, about 3.5-fold, about 4-fold, about 4.5-fold, about 5-fold, about 5.5-fold, about 6-fold, about 6.5-fold, about 7-fold, about 7.5-fold, about 8-fold, about 8.5-fold, about 9-fold, about 9.5-fold, about 10-fold, about 11-fold, about 12-fold, about 13-fold, about 14-fold, about 15-fold, about 16-fold, about 17-fold, about 18-fold, about 19-fold, about 20-fold, about 30-fold, about 40-fold, about 50-fold, about 100-fold, about 500-fold, about 1,000-fold or greater, e.g., from about 1-fold to about 1.5-fold, from about 1.5-fold to about 2-fold, from about 2-fold to about 3-fold, from about 3-fold to about 4-fold, from about 4-fold to about 5-fold, from about 5-fold to about 6-fold, from about 6-fold to about 7-fold, from about 7-fold to about 8-fold, from about 9-fold to about 10-fold, from about 10-fold to about 50-fold, from about 50-fold to about 100-fold, from about 100-fold to about 500-fold, about 500-fold to about 1,000-fold, from about 1-fold to about 10-fold, from about 10-fold to about 100-fold, from about 100-fold to about 1,000-fold, or greater) relative to the reference expression level (e.g., at a time point prior to beginning treatment with an H3K27).

In some embodiments of any of the methods, elevated or increased expression level refers to an overall increase as compared to a reference sample, reference cell, reference tissue, control sample, control cell, control tissue, or internal control (e.g., housekeeping gene).

In some embodiments, the expression level of SMARCA2 is a median expression level (e.g., a median protein expression level or a median gene expression level, e.g., a mean mRNA expression level). Alternatively, the expression level of SMARCA2 can be a mean expression level (e.g., a mean protein expression level or a mean gene expression level, e.g., a mean mRNA expression level).

In some instances, the reference expression level is the expression level of SMARCA2 in a sample obtained from the patient at a previous time point. In other cases, the reference expression level is the expression level of SMARCA2 in a reference population (e.g., a healthy tissue sample from the same patient or a different subject, e.g., a healthy subject, or an average (e.g., mean or median) occupancy level of multiple individuals or patients). In some cases, the reference expression level is a pre-assigned expression level of SMARCA2. For example, a pre-assigned expression level can be statistically or subjectively derived from one or more samples that differ from the sample obtained from the patient as part of a method described herein, e.g., healthy samples, e.g., from the same or different individuals. A reference expression level can be a protein expression level or an mRNA expression level, e.g., according to the type of expression being detected in the patient's sample.

In some embodiments of any of the preceding methods, the occupancy level of H3K27 at a SMARCA2 promoter in a sample (e.g., a tissue sample (e.g., a tumor tissue sample)) obtained from the patient is increased by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 100% or more, about 110% or more, about 120% or more, about 130% or more, about 140% or more, about 150% or more, about 200% or more, about 250% or more, about 300% or more, about 350% or more, about 400% or more, about 450% or more, about 500% or more, about 550% or more, about 600% or more, about 650% or more, about 700% or more, about 750% or more, about 800% or more, about 850% or more, about 900% or more, about 950% or more, about 1,000% or more, about 2,000% or more, about 5,000% or more, or about 10,000% or more, e.g., from about 1% to about 5%, from about 5% to about 10%, from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, from about 95% to about 100%, from about 100% to about 200%, from about 200% to about 300%, from about 300% to about 400%, from about 400% to about 500%, from about 500% to about 600%, from about 600% to about 700%, from about 700% to about 800%, from about 800% to about 1,000%, from about 1,000% to about 2,000%, from about 2,000% to about 5,000%, from about 5,000% to about 10,000%, from about 1% to about 10%, from about 10% to about 20%, from about 20% to about 30%, from about 30% to about 40%, from about 40% to about 50%, from about 50% to about 60%, from about 60% to about 70%, from about 70% to about 80%, from about 80% to about 90%, from about 90% to about 100%, from about 1% to about 25%, from about 25% to about 50%, from about 50% to about 75%, from about 75% to about 100%, from about 1,000% to about 5,000%, or from about 5,000% to about 10,000%) relative to a reference occupancy level. In some embodiments, the occupancy level of H3K27 at a SMARCA2 promoter in a sample is increased (e.g., by about 1-fold, by about 1.1-fold, about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 3-fold, about 3.5-fold, about 4-fold, about 4.5-fold, about 5-fold, about 5.5-fold, about 6-fold, about 6.5-fold, about 7-fold, about 7.5-fold, about 8-fold, about 8.5-fold, about 9-fold, about 9.5-fold, about 10-fold, about 11-fold, about 12-fold, about 13-fold, about 14-fold, about 15-fold, about 16-fold, about 17-fold, about 18-fold, about 19-fold, about 20-fold, about 30-fold, about 40-fold, about 50-fold, about 100-fold, about 500-fold, about 1,000-fold or greater, e.g., from about 1-fold to about 1.5-fold, from about 1.5-fold to about 2-fold, from about 2-fold to about 3-fold, from about 3-fold to about 4-fold, from about 4-fold to about 5-fold, from about 5-fold to about 6-fold, from about 6-fold to about 7-fold, from about 7-fold to about 8-fold, from about 9-fold to about 10-fold, from about 10-fold to about 50-fold, from about 50-fold to about 100-fold, from about 100-fold to about 500-fold, about 500-fold to about 1,000-fold, from about 1-fold to about 10-fold, from about 10-fold to about 100-fold, from about 100-fold to about 1,000-fold, or greater) relative to the reference occupancy level.

In some embodiments of any of the methods, elevated or increased occupancy level refers to an overall increase as compared to a reference sample, reference cell, reference tissue, control sample, control cell, control tissue, or internal control (e.g., housekeeping gene).

Alternatively, the occupancy level of H3K27 at a SMARCA2 promoter in a sample (e.g., a tissue sample (e.g., a tumor tissue sample)) obtained from the patient can be determined to be decreased (e.g., at a time point after the beginning of administration of treatment with an inhibitor of H3K27 methylation relative to a time point prior to the beginning of administration of treatment with an inhibitor of H3K27 methylation). In some embodiments, the occupancy level of H3K27 at a SMARCA2 promoter in a sample is decreased by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 98% or more, about 99% or more, or about 100%, e.g., from about 1% to about 5%, from about 5% to about 10%, from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, from about 90% to about 100%, from about 1% to about 10%, from about 10% to about 20%, from about 20% to about 30%, from about 30% to about 40%, from about 40% to about 50%, from about 50% to about 60%, from about 60% to about 70%, from about 70% to about 80%, from about 80% to about 90%, from about 90% to about 100%, from about 1% to about 25%, from about 25% to about 50%, from about 50% to about 75%, or from about 75% to about 100%)) relative to a reference occupancy level (e.g., obtained from the patient prior to beginning treatment with an inhibitor of H3K27 methylation).

In some embodiments of any of the methods, decreased occupancy level refers to an overall decrease as compared to a reference sample, reference cell, reference tissue, control sample, control cell, control tissue, or internal control (e.g., housekeeping gene).

In some embodiments, the occupancy level of H3K27 (e.g., H3K27me3) at a SMARCA2 promoter is a median occupancy level (e.g., as measured by ChIP-seq or ChIP-PCR). Alternatively, the occupancy level of H3K27 (e.g., H3K27me3) at a SMARCA2 promoter can be a mean occupancy level (e.g., as measured by ChIP-seq or ChIP-PCR).

In some instances, the reference occupancy level is the occupancy level of H3K27 (e.g., H3K27me3) at a SMARCA2 promoter in a sample obtained from the patient at a previous time point. In other cases, the reference occupancy level is the occupancy level of H3K27 (e.g., H3K27me3) at a SMARCA2 promoter in a reference population (e.g., a healthy tissue sample from the same patient or a different subject, e.g., a healthy subject, or an average (e.g., mean or median) occupancy level of multiple individuals or patients). In some cases, the reference occupancy level is a pre-assigned occupancy level of H3K27 (e.g., H3K27me3) at a SMARCA2 promoter. For example, a pre-assigned occupancy level can be statistically or subjectively derived from one or more samples that differ from the sample obtained from the patient as part of a method described herein, e.g., healthy samples, e.g., from the same or different individuals.

In any of the preceding methods, a biomarker (e.g., repressed SMARCA2 relative to a reference level, or high occupancy of H3K27 (e.g., H3K27me3) at a SMARCA2 promoter relative to a reference level) identifies a patient suffering from a cancer having a mutation in one or more genes encoding a SWI/SNF complex protein (e.g., SMARCA4, SMARCB1, SMARCC1, SMARCC2, ARID1A, ARID2, and/or PBRM1) as having an increased likelihood of benefit from treatment with an inhibitor of H3K27 methylation. In some instances, a biomarker (e.g., repressed SMARCA2 or high occupancy of H3K27 (e.g., H3K27me3) at a SMARCA2 promoter relative to a reference level) identifies an ovarian cancer patient as having an increased likelihood of benefit from treatment with an inhibitor of H3K27 methylation. In some instances, a biomarker (e.g., repressed SMARCA2 or high occupancy of H3K27 (e.g., H3K27me3) at a SMARCA2 promoter relative to a reference level) identifies an ovarian clear cell carcinoma patient as having an increased likelihood of benefit from treatment with an inhibitor of H3K27 methylation. In some instances, a biomarker (e.g., repressed SMARCA2 or high occupancy of H3K27 (e.g., H3K27me3) at a SMARCA2 promoter relative to a reference level) identifies a lung cancer patient as having an increased likelihood of benefit from treatment with an inhibitor of H3K27 methylation. In some instances, a biomarker (e.g., repressed SMARCA2 or high occupancy of H3K27 (e.g., H3K27me3) at a SMARCA2 promoter relative to a reference level) identifies a gastric cancer patient as having an increased likelihood of benefit from treatment with an inhibitor of H3K27 methylation. In some instances, a biomarker (e.g., repressed SMARCA2 or high occupancy of H3K27 (e.g., H3K27me3) at a SMARCA2 promoter relative to a reference level) identifies a bladder cancer patient as having an increased likelihood of benefit from treatment with an inhibitor of H3K27 methylation. In some instances, a biomarker (e.g., repressed SMARCA2 or high occupancy of H3K27 (e.g., H3K27me3) at a SMARCA2 promoter relative to a reference level) identifies a rhabdoid cancer patient (e.g., a malignant rhabdoid cancer patient, e.g., a SMARCB1-mutant rhabdoid cancer patient, a renal rhabdoid cancer patient, or a brain rhabdoid cancer patient) as having an increased likelihood of benefit from treatment with an inhibitor of H3K27 methylation. In some instances, a biomarker (e.g., repressed SMARCA2 or high occupancy of H3K27 (e.g., H3K27me3) at a SMARCA2 promoter relative to a reference level) identifies a breast cancer patient as having an increased likelihood of benefit from treatment with an inhibitor of H3K27 methylation. In some instances, a biomarker (e.g., repressed SMARCA2 or high occupancy of H3K27 (e.g., H3K27me3) at a SMARCA2 promoter relative to a reference level) identifies a skin cancer patient as having an increased likelihood of benefit from treatment with an inhibitor of H3K27 methylation.

The presence and/or expression level (amount) of various biomarkers described herein in a sample can be analyzed by a number of methodologies, many of which are known in the art and understood by the skilled artisan, including, but not limited to, immunohistochemistry (“INC”), Western blot analysis, immunoprecipitation, molecular binding assays, enzyme-linked immunosorbent assay (ELISA), enzyme-linked immunofiltration assay (ELIFA), fluorescence activated cell sorting (“FACS”), MassARRAY, proteomics, quantitative blood based assays (e.g., serum ELISA), biochemical enzymatic activity assays, in situ hybridization, fluorescence in situ hybridization (FISH), Southern analysis, Northern analysis, whole genome sequencing, polymerase chain reaction (PCR) (including quantitative real time PCR (qRT-PCR) and other amplification type detection methods, such as, for example, branched DNA, SISBA, TMA and the like), RNA-Seq, microarray analysis, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used. Chromatin modifications, such as histone methylation (e.g., H3K27me3) can be detected and quantified according to known methods (e.g., chromatin immunoprecipitation (ChIP), ChIP-Seq, or ChIP-PCR).

In any of the preceding methods, the presence and/or expression level (amount) of a SMARCA2 may be a nucleic acid expression level. In some instances, the nucleic acid expression level is determined using quantitative polymerase chain reaction (qPCR), reverse transcription PCR (RT-PCR), RNA-Seq, multiplex qPCR or RT-qPCR, microarray analysis, SAGE, MassARRAY technique, or in situ hybridization (e.g., FISH). In some instances, the expression level of a biomarker (e.g., SMARCA2) is determined in tumor tissue, tumor cells, tumor infiltrating immune cells, stromal cells, or combinations thereof.

In a particular instance, the expression level of a biomarker (e.g., SMARCA2) is an mRNA expression level. Methods for the evaluation of mRNAs in cells are well known and include, for example, RNA-Seq (e.g., whole transcriptome shotgun sequencing) using next generation sequencing techniques, hybridization assays using complementary DNA probes (such as in situ hybridization using labeled riboprobes specific for the one or more genes, Northern blot and related techniques) and various nucleic acid amplification assays (such as RT-PCR using complementary primers specific for one or more of the genes, and other amplification type detection methods, such as, for example, branched DNA, SISBA, TMA and the like). In addition, such methods can include one or more steps that allow one to determine the levels of target mRNA in a biological sample (e.g., by simultaneously examining the levels a comparative control mRNA sequence of a “housekeeping” gene such as an actin family member). Optionally, the sequence of the amplified target cDNA can be determined. Optional methods include protocols that examine or detect mRNAs, such as target mRNAs, in a tissue or cell sample by microarray technologies. Using nucleic acid microarrays test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes whose expression correlates with increased or reduced clinical benefit of treatment including an inhibitor of H3K27 methylation may be arrayed on a solid support. Hybridization of a labeled probe with a particular array member indicates that the sample from which the probe was derived expresses that gene.

In any of the preceding methods, the presence and/or expression level (amount) of a biomarker (e.g., SMARCA2 or BRM1) is measured by determining protein expression levels of the biomarker. In certain instances, the method comprises contacting the biological sample with antibodies that specifically bind to a biomarker described herein under conditions permissive for binding of the biomarker, and detecting whether a complex is formed between the antibodies and biomarker. Such a method may be an in vitro or in vivo method. Any method of measuring protein expression levels known in the art may be used. For example, in some instances, a protein expression level of a biomarker (e.g., SMARCA2 or BRM1) is determined using a method selected from the group consisting of flow cytometry (e.g., fluorescence-activated cell sorting (FACS™)), Western blot, ELISA, ELIFA, immunoprecipitation, immunohistochemistry (INC), immunofluorescence, radioimmunoassay, dot blotting, immunodetection methods, HPLC, surface plasmon resonance, optical spectroscopy, mass spectrometry, and HPLC. In some instances, the protein expression level of the biomarker (e.g., SMARCA2 or BRM1) is determined in tumor cells (e.g., from a biopsy).

In certain embodiments, the presence and/or expression level/amount of a biomarker protein (e.g., PD-L1) in a sample is examined using IHC and staining protocols. IHC staining of tissue sections has been shown to be a reliable method of determining or detecting the presence of proteins in a sample. In some embodiments of any of the methods, assays and/or kits, the biomarker is BMR1.

IHC may be performed in combination with additional techniques such as morphological staining and/or in situ hybridization (e.g., FISH). Two general methods of IHC are available; direct and indirect assays. According to the first assay, binding of antibody to the target antigen is determined directly. This direct assay uses a labeled reagent, such as a fluorescent tag or an enzyme-labeled primary antibody, which can be visualized without further antibody interaction. In a typical indirect assay, unconjugated primary antibody binds to the antigen and then a labeled secondary antibody binds to the primary antibody. Where the secondary antibody is conjugated to an enzymatic label, a chromogenic or fluorogenic substrate is added to provide visualization of the antigen. Signal amplification occurs because several secondary antibodies may react with different epitopes on the primary antibody.

In some embodiments, the presence of a biomarker (e.g., BRM1) is detected by IHC in >0% of the sample, in at least 1% of the sample, in at least 5% of the sample, in at least 10% of the sample, in at least 15% of the sample, in at least 15% of the sample, in at least 20% of the sample, in at least 25% of the sample, in at least 30% of the sample, in at least 35% of the sample, in at least 40% of the sample, in at least 45% of the sample, in at least 50% of the sample, in at least 55% of the sample, in at least 60% of the sample, in at least 65% of the sample, in at least 70% of the sample, in at least 75% of the sample, in at least 80% of the sample, in at least 85% of the sample, in at least 90% of the sample, in at least 95% of the sample, or more. Samples may be scored using known methods, for example, by a pathologist or automated image analysis.

In some embodiments, a method of the invention includes identifying a mutation in one or more genes encoding a nucleosome remodeling protein (e.g., a SWI/SNF family protein or a SWI/SNF complex protein, e.g., a gene encoding BRG1, SNF5, INI1, or BAF, e.g., SMARCA4, SMARCB1, SMARCC1, SMARCC2, ARID1A, ARID2, and PBRM1). In some embodiments, a mutation in one or more genes encoding a nucleosome remodeling protein identifies a patient as having a greater likelihood of having a decreased (repressed) expression level of SMARCA2 and/or an increased (elevated) occupancy level of H3K27 (e.g., H3K27me3) at a SMARCA2 promoter in a sample.

A mutation can be identified according to methods known in the art and described herein. In some embodiments, a mutation (e.g., a mutation in one or more genes encoding a nucleosome remodeling protein, e.g., a SWI/SNF family protein or a SWI/SNF complex protein, e.g., BRG1, SNF5 (IN11), SWI/SNF complex 155-kDa subunit, SWI/SNF complex-170 kDa subunit, or BAF, zipzap protein, or BAF180, or a protein encoded by any one of SMARCA4, SMARCB1, SMARCC1, SMARCC2, ARID1A, ARID2, and PBRM1) is identified by determining a nucleic acid sequence (e.g., a DNA sequence or an RNA sequence) in a sample obtained from a patient and comparing the sequence to a reference sequence (e.g., a wildtype sequence).

In certain instances, a reference level, reference sample, reference cell, reference tissue, control sample, control cell, or control tissue is a single sample or a combination of multiple samples from the same subject or individual that are obtained at one or more different time points than when the test sample is obtained. For example, a reference level, reference sample, reference cell, reference tissue, control sample, control cell, or control tissue is obtained at an earlier time point from the same subject or individual than when the test sample is obtained. Such reference level, reference sample, reference cell, reference tissue, control sample, control cell, or control tissue may be useful if the reference sample is obtained during initial diagnosis of cancer and the test sample is later obtained when the cancer becomes metastatic.

In certain embodiments, a reference level, reference sample, reference cell, reference tissue, control sample, control cell, or control tissue is a combination of multiple samples from one or more healthy individuals who are not the patient. In certain embodiments, a reference level, reference sample, reference cell, reference tissue, control sample, control cell, or control tissue is a combination of multiple samples from one or more individuals with a disease or disorder (e.g., cancer) who are not the patient or individual. In certain embodiments, a reference level, reference sample, reference cell, reference tissue, control sample, control cell, or control tissue is pooled RNA samples from normal tissues or pooled plasma or serum samples from one or more individuals who are not the patient. In certain embodiments, a reference level, reference sample, reference cell, reference tissue, control sample, control cell, or control tissue is pooled RNA samples from tumor tissues or pooled plasma or serum samples from one or more individuals with a disease or disorder (e.g., cancer) who are not the patient. In certain embodiments, the reference level is the median level of expression of a biomarker across a set of samples (e.g., a set of tissue samples (e.g., a set of tumor tissue samples)). In certain embodiments, the reference level is the median level of expression of a biomarker across a population of patients having a particular disease or disorder (e.g., a proliferative cell disorder (e.g., a cancer)).

In some embodiments, the sample obtained from the patient is collected after the beginning of an anti-cancer therapy, e.g., therapy for the treatment of cancer or the management or amelioration of a symptom thereof. Therefore, in some embodiments, the sample is collected after the administration of chemotherapeutics or the start of a chemotherapy regimen.

In some embodiments of any of the previous methods, the provides methods for identifying a patient having a cancer who may benefit from treatment including one or more inhibitors of H3K27 methylation, optimizing therapeutic efficacy for treatment of a patient having cancer, predicting responsiveness of a patient having a cancer to treatment including one or more inhibitors of H3K27 methylation, and selecting a therapy for a patient having a cancer, based on determining an expression level of SMARCA2 or an occupancy level of H3K27 (e.g., H3K27me3) at a SMARCA2 promoter, wherein the sample also includes a mutation in one or more genes encoding a nucleosome remodeling protein. Therefore, the methods of the invention further provide a method of identifying a mutation in one or more genes encoding a nucleosome remodeling protein (e.g., a SWI/SNF family protein, e.g., BRG1, SNF5 (IN11), SWI complex 155-kDa subunit, SWI complex 170-kDa subunit, BAF, zipzap protein, or BAF180). Genes that encode a nucleosome remodeling protein include, but are not limited to, SMARCA4, SMARCB1, SMARCC1, SMARCC2, ARID1A, ARID2, and PBRM1. In some cases, a cancer having a mutation in one or more genes encoding a nucleosome remodeling protein (e.g., one or more genes encoding a SWI/SNF family protein, e.g., SMARCA4, SMARCB1, SMARCC1, SMARCC2, ARID1A, ARID2, or PBRM1) identifies a patient who is more likely to have a decreased expression level of SMARCA2 and/or an increased occupancy level of H3K27 (e.g., H3K27me3) at a SMARCA2 promoter.

B. Treatment with Inhibitors of H3K27 Methylation

The present invention provides methods for treating a patient having a cancer (e.g., rhabdoid cancer (e.g., malignant rhabdoid cancer (e.g., malignant rhabdoid brain cancer or malignant rhabdoid renal cancer), ovarian cancer (e.g., ovarian clear cell carcinoma, or a small cell carcinoma of the ovary, e.g., a small cell carcinoma of the ovary, hypercalcemic type), lung cancer, gastric cancer, bladder cancer, breast cancer, skin cancer, colorectal cancer, stomach cancer, lymphoid cancer, cervical cancer, peritoneal cancer, pancreatic cancer, glioblastoma, liver cancer, bladder cancer, colon cancer, endometrial cancer, uterine cancer, renal cancer, prostate cancer, thyroid cancer, and head and neck cancer). In some instances, the methods of the invention include administering to the patient an inhibitor of H3K27 methylation. Any of the inhibitors of H3K27 methylation described herein or known in the art may be used in connection with any of the methods of the invention.

In some instances, the methods involve determining the expression level of SMARCA2 in a sample obtained from a patient and administering a therapy including one or more inhibitors of H3K27 methylation to the patient based a decreased expression level of SMARCA2 in the sample as compared to a reference level. In some instances, administering an inhibitor of H3K27 methylation is after the expression level of SMARCA2 has been determined to be decreased relative to a reference level. In some instances, a patient currently being treated with an inhibitor of H3K27 methylation may continue to receive treatment including an inhibitor of H3K27 methylation following a determination that the expression level of SMARCA2 is decreased relative to a reference level.

In some instances, the methods involve determining the occupancy level of H3K27 (e.g., H3K27 mono-, di-, or trimethylation; e.g., H3K27me3) at a SMARCA2 promoter in a sample obtained from a patient and administering a therapy including one or more inhibitors of H3K27 methylation to the patient based an increased occupancy level of H3K27 (e.g., H3K27me3) at the SMARCA2 promoter in the sample as compared to a reference level. In some instances, administering an inhibitor of H3K27 methylation is after the occupancy level of H3K27 (e.g., H3K27me3) at the SMARCA2 promoter has been determined to be increased relative to a reference level. In some instances, a patient currently being treated with an inhibitor of H3K27 methylation may continue to receive treatment including an inhibitor of H3K27 methylation following a determination that the occupancy level of H3K27 (e.g., H3K27me3) at the SMARCA2 promoter is increased relative to a reference level.

In any of the preceding methods, one or more inhibitors of H3K27 methylation may be administered when the expression level of SMARCA2 in a sample (e.g., a tissue sample (e.g., a tumor tissue sample)) obtained from the patient has been determined to be decreased by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 98% or more, about 99% or more, or about 100%) relative to a reference expression level.

In some embodiments, the expression level of SMARCA2 is a median expression level (e.g., a median protein expression level or a median gene expression level, e.g., a mean mRNA expression level). Alternatively, the expression level of SMARCA2 can be a mean expression level (e.g., a mean protein expression level or a mean gene expression level, e.g., a mean mRNA expression level).

In some instances, the reference expression level is the expression level of SMARCA2 in a sample obtained from the patient at a previous time point. In other cases, the reference expression level is the expression level of SMARCA2 in a reference population (e.g., a healthy tissue sample from the same patient or a different subject, e.g., a healthy subject, or an average (e.g., mean or median) occupancy level of multiple individuals or patients). In some cases, the reference expression level is a pre-assigned expression level of SMARCA2. For example, a pre-assigned expression level can be statistically or subjectively derived from one or more samples that differ from the sample obtained from the patient as part of a method described herein, e.g., healthy samples, e.g., from the same or different individuals. A reference expression level can be a protein expression level or an mRNA expression level, e.g., according to the type of expression being detected in the patient's sample.

In some embodiments of any of the preceding methods, one or more inhibitors of H3K27 methylation may be administered when the occupancy level of H3K27 (e.g., H3K27me3) at a SMARCA2 promoter in a sample (e.g., a tissue sample (e.g., a tumor tissue sample)) obtained from the patient has been determined to be increased by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 100% or more, about 110% or more, about 120% or more, about 130% or more, about 140% or more, about 150% or more, about 200% or more, about 250% or more, about 300% or more, about 350% or more, about 400% or more, about 450% or more, about 500% or more, about 550% or more, about 600% or more, about 650% or more, about 700% or more, about 750% or more, about 800% or more, about 850% or more, about 900% or more, about 950% or more, about 1,000% or more, about 2,000% or more, about 5,000% or more, or about 10,000% or more) relative to a reference occupancy level.

In some embodiments, the occupancy level of H3K27 (e.g., H3K27me3) at a SMARCA2 promoter is a median occupancy level (e.g., as measured by ChIP-seq or ChIP-PCR). Alternatively, the occupancy level of H3K27 (e.g., H3K27me3) at a SMARCA2 promoter can be a mean occupancy level (e.g., as measured by ChIP-seq or ChIP-PCR).

In some instances, the reference occupancy level is the occupancy level of H3K27 (e.g., H3K27me3) at a SMARCA2 promoter in a sample obtained from the patient at a previous time point. In other cases, the reference occupancy level is the occupancy level of H3K27 (e.g., H3K27me3) at a SMARCA2 promoter in a reference population (e.g., a healthy tissue sample from the same patient or a different subject, e.g., a healthy subject, or an average (e.g., mean or median) occupancy level of multiple individuals or patients). In some cases, the reference occupancy level is a pre-assigned occupancy level of H3K27 (e.g., H3K27me3) at a SMARCA2 promoter. For example, a pre-assigned occupancy level can be statistically or subjectively derived from one or more samples that differ from the sample obtained from the patient as part of a method described herein, e.g., healthy samples, e.g., from the same or different individuals.

In certain embodiments, the method includes administering to a patient suffering from a cancer having a mutation in one or more genes encoding a SWI/SNF complex protein (e.g., SMARCA4, SMARCB1, SMARCC1, SMARCC2, ARID1A, ARID2, and/or PBRM1) an inhibitor of H3K27 methylation (e.g., an H3K27 inhibitor, e.g., an EZH2 inhibitor, e.g., EPZ-6438, CPI-169, EPZ005687, GSK-126, GSK343, or GSK503) when a decreased level of expression of SMARCA2 relative to a reference expression level identifies the patient as having an increased likelihood of benefit from treatment with an inhibitor of H3K27 methylation. In some instances, the method includes administering to an ovarian cancer patient an inhibitor of H3K27 methylation (e.g., an EZH2 inhibitor) when a decreased level of expression of SMARCA2 relative to a reference expression level identifies the patient as having an increased likelihood of benefit from treatment with an inhibitor of H3K27 methylation. In some instances, the method includes administering to an ovarian clear cell carcinoma patient an inhibitor of H3K27 methylation (e.g., an EZH2 inhibitor) when a decreased level of expression of SMARCA2 relative to a reference expression level identifies the patient as having an increased likelihood of benefit from treatment with an inhibitor of H3K27 methylation. In some instances, the method includes administering to a lung cancer patient an inhibitor of H3K27 methylation (e.g., an EZH2 inhibitor) when a decreased level of expression of SMARCA2 relative to a reference expression level identifies the patient as having an increased likelihood of benefit from treatment with an inhibitor of H3K27 methylation. In some instances, the method includes administering to a gastric cancer patient an inhibitor of H3K27 methylation (e.g., an EZH2 inhibitor) when a decreased level of expression of SMARCA2 relative to a reference expression level identifies the patient as having an increased likelihood of benefit from treatment with an inhibitor of H3K27 methylation. In some instances, the method includes administering to a bladder cancer patient an inhibitor of H3K27 methylation (e.g., an EZH2 inhibitor) when a decreased level of expression of SMARCA2 relative to a reference expression level identifies the patient as having an increased likelihood of benefit from treatment with an inhibitor of H3K27 methylation. In some instances, the method includes administering to a rhabdoid cancer patient (e.g., a malignant rhabdoid cancer patient, e.g., a SMARCB1-mutant rhabdoid cancer patient) an inhibitor of H3K27 methylation (e.g., an EZH2 inhibitor) when a decreased level of expression of SMARCA2 relative to a reference expression level identifies the patient as having an increased likelihood of benefit from treatment with an inhibitor of H3K27 methylation. In some instances, the method includes administering to a breast cancer patient an inhibitor of H3K27 methylation (e.g., an EZH2 inhibitor) when a decreased level of expression of SMARCA2 relative to a reference expression level identifies the patient as having an increased likelihood of benefit from treatment with an inhibitor of H3K27 methylation. In some instances, the method includes administering to a skin cancer patient an inhibitor of H3K27 methylation (e.g., an EZH2 inhibitor) when a decreased level of expression of SMARCA2 relative to a reference expression level identifies the patient as having an increased likelihood of benefit from treatment with an inhibitor of H3K27 methylation.

In any of the above methods, administration of one or more inhibitors of H3K27 methylation can have the therapeutic effect (i.e., benefit) of a cellular or biological response, a complete response, a partial response, a stable disease (without progression or relapse), or a response with a later relapse of the patient from or as a result of the treatment with the inhibitor of H3K27 methylation. For example, an effective response can be reduced tumor size (volume), increased progression-free survival (PFS), and/or increased overall survival (OS) in a patient diagnosed as (i) expressing a decreased level of SMARCA2 compared to a reference level or (ii) having an increased occupancy level of H3K27 (e.g., H3K27me3) at a SMARCA2 promoter compared to a reference level. In some instances, administration of an inhibitor of H3K27 methylation has a therapeutic effect of a reduction in tumor size (volume) by 1% or more (e.g., 2%, 3%, 4%, 5%, 6%, 7%, 8%, 9% 10%, 20%, 30%, 40%, 50%, 60%, 70%, 80%, or 90% or more). The decreased expression of SMARCA2 and/or increased occupancy level of H3K27 (e.g., H3K27me3) at a SMARCA2 promoter predicts such therapeutic efficacy. In some instances, administration of an inhibitor of H3K27 methylation has the therapeutic effect of increasing progression-free survival (PFS) by 1 day or more (e.g., by 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, or 1 year or more).

### Inhibitors of H3K27 Methylation for Use in the Methods of the Invention

Provided herein are methods for treating or delaying the progression of a proliferative cell disorder (e.g., cancer (e.g., rhabdoid cancer (e.g., malignant rhabdoid cancer (e.g., malignant rhabdoid brain cancer or malignant rhabdoid renal cancer)), ovarian cancer (e.g., ovarian clear cell carcinoma, or a small cell carcinoma of the ovary, e.g., a small cell carcinoma of the ovary, hypercalcemic type), lung cancer, gastric cancer, bladder cancer, breast cancer, skin cancer, colorectal cancer, stomach cancer, lymphoid cancer, cervical cancer, peritoneal cancer, pancreatic cancer, glioblastoma, liver cancer, bladder cancer, colon cancer, endometrial cancer, uterine cancer, renal cancer, prostate cancer, thyroid cancer, and head and neck cancer)) in a patient comprising administering to the patient a therapeutically effective amount of one or more inhibitors of H3K27 methylation.

In some embodiments, an inhibitor of H3K27 methylation may inhibit the activity of one or more proteins involved in the methylation (e.g., monomethylation, dimethylation, or trimethylation) of H3K27. In some embodiments, the inhibitor of H3K27 methylation is an agent that disrupts the formation or activity of a polycomb repressive complex 2 (PRC2). For example, an inhibitor of H3K27 methylation may disrupt the formation or activity of PRC2 by antagonizing or reducing, blocking, or inhibiting expression of one or more of SUZ12, EED, RBAP, and/or JARID2. In some embodiments, the inhibitor of H3K27 methylation may be a small molecule (e.g., a small molecule H3K27me3 inhibitor, e.g., an EZH2 inhibitor). In some embodiments, the inhibitor of H3K27 methylation may be a protein (e.g., a peptide). In some embodiments, the inhibitor of H3K27 methylation may be an antibody, an antigen binding fragment thereof, an immunoadhesin, a fusion protein, an oligopeptide, or an aptamer.

In some embodiments, an inhibitor of H3K27 methylation is an EZH2 inhibitor. An EZH2 inhibitor is a molecule that decreases, blocks, inhibits, abrogates, or interferes with the methyltransferase activity of EZH2. In some embodiments, an EZH2 inhibitor is a small molecule. Examples of small molecule inhibitors of EZH2 include, but are not limited to, EPZ-6438, CPI-169, EPZ005687, GSK-126, GSK343, GSK503, and pharmaceutically acceptable salts thereof. EZH2 inhibitors may inhibit only EZH2 or may inhibit EZH2 and one or more additional targets. In some embodiments, EZH2 inhibitors preferentially inhibit EZH2 in comparison with EZH1.

### Dosage and Administration

Once a patient responsive or sensitive to treatment with an inhibitor of H3K27 methylation has been identified, treatment with the inhibitor of H3K27 methylation, alone or in combination with other therapeutic agents, can be carried out. Such treatment may result in, for example, a reduction in tumor size or an increase in progression-free survival (PFS) and/or overall survival (OS). Moreover, treatment with the combination of an inhibitor of H3K27 methylation and at least one additional therapeutic agent preferably results in an additive, more preferably synergistic (or greater than additive), therapeutic benefit to the patient. Preferably, in this combination method the timing between at least one administration of the inhibitor of H3K27 methylation and at least one additional therapeutic agent is about one month or less, and more preferably, about two weeks or less.

It will be appreciated by those of skill in the art that the exact manner of administering a therapeutically effective amount of an inhibitor of H3K27 methylation to a patient following diagnosis of their likely responsiveness to the inhibitor of H3K27 methylation will be at the discretion of the attending physician. The mode of administration, including dosage, combination with other agents, timing and frequency of administration, and the like, may be affected by the diagnosis of a patient's likely responsiveness to such inhibitor of H3K27 methylation, as well as the patient's condition and history. Thus, even patients having cancers who are predicted to be relatively insensitive to an inhibitor of H3K27 methylation may still benefit from treatment therewith, particularly in combination with other agents, including agents that may alter a patient's responsiveness to the antagonist.

A composition comprising an inhibitor of H3K27 methylation will be formulated, dosed, and administered in a fashion consistent with good medical practice. Factors for consideration in this context include the particular type of cancer being treated (e.g., rhabdoid cancer (e.g., malignant rhabdoid cancer (e.g., malignant rhabdoid brain cancer or malignant rhabdoid renal cancer)), ovarian cancer (e.g., ovarian clear cell carcinoma, or a small cell carcinoma of the ovary, e.g., a small cell carcinoma of the ovary, hypercalcemic type), lung cancer, gastric cancer, bladder cancer, breast cancer, skin cancer, colorectal cancer, stomach cancer, lymphoid cancer, cervical cancer, peritoneal cancer, pancreatic cancer, glioblastoma, liver cancer, bladder cancer, colon cancer, endometrial cancer, uterine cancer, renal cancer, prostate cancer, thyroid cancer, and head and neck cancer), the particular mammal being treated (e.g., human), the clinical condition of the individual patient, the cause of the cancer, the site of delivery of the agent, possible side-effects, the type of inhibitor, the method of administration, the scheduling of administration, and other factors known to medical practitioners. The effective amount of the inhibitor of H3K27 methylation to be administered will be governed by such considerations.

A physician having ordinary skill in the art can readily determine and prescribe the effective amount of the pharmaceutical composition required, depending on such factors as the particular antagonist type. For example, the physician could start with doses of such an inhibitor of H3K27 methylation, employed in the pharmaceutical composition at levels lower than that required in order to achieve the desired therapeutic effect and gradually increase the dosage until the desired effect is achieved. The effectiveness of a given dose or treatment regimen of the antagonist can be determined, for example, by assessing signs and symptoms in the patient using standard measures of efficacy.

In certain examples, the inhibitor of H3K27 methylation may be the only agent administered to the subject (i.e., as a monotherapy).

In certain examples, the patient is treated with the same inhibitor of H3K27 methylation at least twice. Thus, the initial and second inhibitor of H3K27 methylation exposures may be with the same inhibitor or, alternatively, all inhibitor of H3K27 methylation exposures are with the same inhibitor of H3K27 methylation, i.e., treatment for the first two exposures, and preferably all exposures, is with one type of inhibitor of H3K27 methylation.

Treatment with inhibitors of H3K27 methylation, or pharmaceutically acceptable salts thereof, can be carried out according to standard methods.

If multiple exposures of an inhibitor of H3K27 methylation are provided, each exposure may be provided using the same or a different administration means. In one embodiment, each exposure is given by oral administration. In one embodiment, each exposure is by intravenous administration. In another embodiment, each exposure is given by subcutaneous administration. In yet another embodiment, the exposures are given by both intravenous and subcutaneous administration.

The duration of therapy can be continued for as long as medically indicated or until a desired therapeutic effect (e.g., those described herein) is achieved. In certain embodiments, the therapy is continued for 1 month, 2 months, 4 months, 6 months, 8 months, 10 months, 1 year, 2 years, 3 years, 4 years, 5 years, or fora period of years up to the lifetime of the subject.

As noted above, however, these suggested amounts of inhibitors of H3K27 methylation are subject to a great deal of therapeutic discretion. The key factor in selecting an appropriate dose and scheduling is the result obtained, as indicated above. In some embodiments, the inhibitor of H3K27 methylation is administered as close to the first sign, diagnosis, appearance, or occurrence of the proliferative cell disorder (e.g., cancer) as possible.

Routes of Administration

Inhibitors of H3K27 methylation and any additional therapeutic agents may be formulated, dosed, and administered in a fashion consistent with good medical practice. Factors for consideration in this context include the particular disorder being treated (e.g., cancer), the particular mammal being treated, the clinical condition of the individual patient, the cause of the disorder, the site of delivery of the agent, the method of administration, the scheduling of administration, and other factors known to medical practitioners. The inhibitor of H3K27 methylation need not be, but is optionally formulated with and/or administered concurrently with, one or more agents currently used to prevent or treat the disorder (e.g., cancer).

For the prevention or treatment of a cancer, the appropriate dosage of an inhibitor of H3K27 methylation described herein (when used alone or in combination with one or more other additional therapeutic agents) will depend on the type of disease to be treated, the severity and course of the disease, whether the inhibitor of H3K27 methylation is administered for preventive or therapeutic purposes, previous therapy, the patient's clinical history and response to the inhibitor of H3K27 methylation, and the discretion of the attending physician. The inhibitor of H3K27 methylation is suitably administered to the patient at one time or over a series of treatments. For repeated administrations over several days or longer, depending on the condition, the treatment would generally be sustained until a desired suppression of disease symptoms occurs. Such doses may be administered intermittently, e.g., every week or every three weeks (e.g., such that the patient receives, for example, from about two to about twenty, or e.g., about six doses of the inhibitor of H3K27 methylation). An initial higher loading dose, followed by one or more lower doses may be administered. However, other dosage regimens may be useful. The progress of this therapy is easily monitored by conventional techniques and assays.

The inhibitor of H3K27 methylation can be administered by any suitable means, including orally, parenteral, topical, subcutaneous, intraperitoneal, intrapulmonary, intranasal, and/or intralesional administration. Parenteral infusions include intramuscular, intravenous, intraarterial, intraperitoneal, or subcutaneous administration. Intrathecal administration is also contemplated. In addition, the inhibitor of H3K27 methylation may suitably be administered by pulse infusion, e.g., with declining doses of the inhibitor of H3K27 methylation. Optionally, the dosing is given by oral administration.

If multiple exposures of an inhibitor of H3K27 methylation are provided, each exposure may be provided using the same or a different administration means. In one embodiment, each exposure is by oral administration. For example, one or more inhibitors of H3K27 methylation, such as EPZ-6438, CPI-169, EPZ005687, GSK-126, GSK343, and/or GSK503, can provided in tablet form. For example, one or more inhibitors of H3K27 methylation, such as EPZ-6438, CPI-169, EPZ005687, GSK-126, GSK343, and/or GSK503, can be administered twice a day. In another embodiment, each exposure is given intravenously (i.v.). In another embodiment, each exposure is given by subcutaneous (s.c.) administration. In yet another embodiment, the exposures are given by both i.v. and s.c. administration.

Combination Therapy

Any of the preceding methods may include administration of more than one therapeutic agent. In some cases, the invention provides a method of treating an individual by administering a first inhibitor of H3K27 methylation and a second (e.g., different) inhibitor of H3K27 methylation. In other cases, the invention provides a method of treating an individual by administering one or more inhibitors of H3K27 methylation in combination with an additional (e.g., different) therapeutic agent (e.g., an anti-cancer agent).

In some instances, the method includes administering an anti-cancer agent, such as a chemotherapeutic agent, a growth-inhibitory agent, a biotherapy, an immunotherapy, or a radiation therapy agent. In addition, cytotoxic agents, anti-angiogenic, and anti-proliferative agents can be used in combination with the inhibitor of H3K27 methylation. In some instances, the inhibitor of H3K27 methylation is used in combination with an anti-cancer therapy, such as surgery.

The combination therapy may provide “synergy” and prove “synergistic,” i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially. In general, during alternation therapy, an effective dosage of each active ingredient is administered sequentially (i.e., serially), whereas in combination therapy, effective dosages of two or more active ingredients are administered together.

As described above, the therapeutic methods may include administering a combination of two or more (e.g., three or more) inhibitors of H3K27 methylation (e.g., EZH2 inhibitors, e.g., EPZ-6438, CPI-169, EPZ005687, GSK-126, GSK343, and/or GSK503). In some instances, an inhibitor of H3K27 methylation, e.g., an H3K27me3 inhibitor (e.g., EZH2 inhibitors, e.g., EPZ-6438, CPI-169, EPZ005687, GSK-126, GSK343, or GSK503) is administered in combination with an agent that disrupts the formation or activity of PCR2. In some instances, an inhibitor of H3K27 methylation, e.g., an H3K27me3 inhibitor (e.g., EZH2 inhibitors, e.g., EPZ-6438, CPI-169, EPZ005687, GSK-126, GSK343, or GSK503) is administered in combination with SUZ12 antagonist. In some instances, an inhibitor of H3K27 methylation, e.g., an H3K27me3 inhibitor (e.g., EZH2 inhibitors, e.g., EPZ-6438, CPI-169, EPZ005687, GSK-126, GSK343, or GSK503) is administered in combination with EED antagonist. In some instances, an inhibitor of H3K27 methylation, e.g., an H3K27me3 inhibitor (e.g., EZH2 inhibitors, e.g., EPZ-6438, CPI-169, EPZ005687, GSK-126, GSK343, or GSK503) is administered in combination with RBAP antagonist. In some instances, an inhibitor of H3K27 methylation, e.g., an H3K27me3 inhibitor (e.g., EZH2 inhibitors, e.g., EPZ-6438, CPI-169, EPZ005687, GSK-126, GSK343, or GSK503) is administered in combination with JARID2 antagonist. In some instances, an inhibitor of H3K27 methylation, e.g., an H3K27me3 inhibitor (e.g., EZH2 inhibitors, e.g., EPZ-6438, CPI-169, EPZ005687, GSK-126, GSK343, or GSK503) is administered in combination with an agent that reduces the expression of SUZ12. In some instances, an inhibitor of H3K27 methylation, e.g., an H3K27me3 inhibitor (e.g., EZH2 inhibitors, e.g., EPZ-6438, CPI-169, EPZ005687, GSK-126, GSK343, or GSK503) is administered with an agent that reduces the expression of EED. In some instances, an inhibitor of H3K27 methylation, e.g., an H3K27me3 inhibitor (e.g., EZH2 inhibitors, e.g., EPZ-6438, CPI-169, EPZ005687, GSK-126, GSK343, or GSK503) is administered with an agent that reduces the expression of jumonji.

The methods may also involve administering to the patient an effective amount of an inhibitor of H3K27 methylation in combination with a chemotherapeutic agent, such as docetaxel, doxorubicin, and cyclophosphamide.

In other instances, the method includes administering an inhibitor of H3K27 methylation in combination with an immunotherapeutic, such as a therapeutic antibody. In one embodiment, the therapeutic antibody is an antibody that binds a cancer cell surface marker or tumor associated-antigen (TAA). In one embodiment, the therapeutic antibody is an anti-HER2 antibody, trastuzumab (e.g., HERCEPTIN®). In one embodiment, the therapeutic antibody is an anti-HER2 antibody, pertuzumab (OMNITARG™). In another embodiment, the therapeutic antibody either a naked antibody or an antibody-drug conjugate (ADC).

Without wishing to be bound to theory, it is thought that enhancing T-cell stimulation, by promoting an activating co-stimulatory molecule or by inhibiting a negative co-stimulatory molecule, may promote tumor cell death thereby treating or delaying progression of cancer. Therefore, in some instances, an inhibitor of H3K27 methylation may be administered in conjunction with an agonist directed against an activating co-stimulatory molecule. In some instances, an activating co-stimulatory molecule may include CD40, CD226, CD28, OX40, GITR, CD137, CD27, HVEM, or CD127. In some instances, the agonist directed against an activating co-stimulatory molecule is an agonist antibody that binds to CD40, CD226, CD28, OX40, GITR, CD137, CD27, HVEM, or CD127. In some instances, an inhibitor of H3K27 methylation may be administered in conjunction with an antagonist directed against an inhibitory co-stimulatory molecule. In some instances, an inhibitory co-stimulatory molecule may include CTLA-4 (also known as CD152), TIM-3, BTLA, VISTA, LAG-3, B7-H3, B7-H4, IDO, TIGIT, MICA/B, or arginase. In some instances, the antagonist directed against an inhibitory co-stimulatory molecule is an antagonist antibody that binds to CTLA-4, TIM-3, BTLA, VISTA, LAG-3, B7-H3, B7-H4, IDO, TIGIT, MICA/B, or arginase.

In some instances, an inhibitor of H3K27 methylation may be administered in conjunction with an antagonist directed against CTLA-4 (also known as CD152), e.g., a blocking antibody. In some instances, an inhibitor of H3K27 methylation may be administered in conjunction with ipilimumab (also known as MDX-010, MDX-101, or YERVOY®). In some instances, an inhibitor of H3K27 methylation may be administered in conjunction with tremelimumab (also known as ticilimumab or CP-675,206). In some instances, an inhibitor of H3K27 methylation may be administered in conjunction with an antagonist directed against B7-H3 (also known as CD276), e.g., a blocking antibody. In some instances, an inhibitor of H3K27 methylation may be administered in conjunction with MGA271. In some instances, an inhibitor of H3K27 methylation may be administered in conjunction with an antagonist directed against a TGF-β, e.g., metelimumab (also known as CAT-192), fresolimumab (also known as GC1008), or LY2157299.

In some instances, an inhibitor of H3K27 methylation may be administered in conjunction with a treatment including adoptive transfer of a T cell (e.g., a cytotoxic T cell or cytotoxic lymphocyte (CTL)) expressing a chimeric antigen receptor (CAR). In some instances, an inhibitor of H3K27 methylation may be administered in conjunction with a treatment including adoptive transfer of a T cell including a dominant-negative TGF-β receptor, e.g., a dominant-negative TGF-β type II receptor. In some instances, an inhibitor of H3K27 methylation may be administered in conjunction with a treatment including a HERCREEM protocol (see, e.g., ClinicalTrials.gov Identifier NCT00889954).

In some instances, an inhibitor of H3K27 methylation may be administered in conjunction with an agonist directed against CD137 (also known as TNFRSF9, 4-1BB, or ILA), e.g., an activating antibody. In some instances, an inhibitor of H3K27 methylation may be administered in conjunction with urelumab (also known as BMS-663513). In some instances, an inhibitor of H3K27 methylation may be administered in conjunction with an agonist directed against CD40, e.g., an activating antibody. In some instances, an inhibitor of H3K27 methylation may be administered in conjunction with CP-870893. In some instances, an inhibitor of H3K27 methylation may be administered in conjunction with an agonist directed against OX40 (also known as CD134), e.g., an activating antibody. In some instances, an inhibitor of H3K27 methylation may be administered in conjunction with an anti-OX40 antibody (e.g., AgonOX). In some instances, an inhibitor of H3K27 methylation may be administered in conjunction with an agonist directed against CD27, e.g., an activating antibody. In some instances, an inhibitor of H3K27 methylation may be administered in conjunction with CDX-1127. In some instances, an inhibitor of H3K27 methylation may be administered in conjunction with an antagonist directed against indoleamine-2,3-dioxygenase (IDO). In some instances, with the IDO antagonist is 1-methyl-D-tryptophan (also known as 1-D-MT). In some instances, an inhibitor of H3K27 methylation may be administered in conjugation with a PD-1 axis binding antagonist. In some instances, the PD-1 axis binding antagonist is a PD-L1 antibody.

In some instances, an inhibitor of H3K27 methylation may be administered in conjunction with an antibody-drug conjugate. In some instances, the antibody-drug conjugate comprises mertansine or monomethyl auristatin E (MMAE). In some instances, an inhibitor of H3K27 methylation may be administered in conjunction with an anti-NaPi2b antibody-MMAE conjugate (also known as DNIB0600A or RG7599). In some instances, an inhibitor of H3K27 methylation may be administered in conjunction with trastuzumab emtansine (also known as T-DM1, ado-trastuzumab emtansine, or KADCYLA®, Genentech). In some instances, an inhibitor of H3K27 methylation may be administered in conjunction with DMUC5754A. In some instances, an inhibitor of H3K27 methylation may be administered in conjunction with an antibody-drug conjugate targeting the endothelin B receptor (EDNBR), e.g., an antibody directed against EDNBR conjugated with MMAE.

In some instances, an inhibitor of H3K27 methylation may be administered in conjunction with an anti-angiogenesis agent. In some instances, an inhibitor of H3K27 methylation may be administered in conjunction with an antibody directed against a VEGF, e.g., VEGF-A. In some instances, an inhibitor of H3K27 methylation may be administered in conjunction with bevacizumab (also known as AVASTIN®, Genentech). In some instances, an inhibitor of H3K27 methylation may be administered in conjunction with an antibody directed against angiopoietin 2 (also known as Ang2). In some instances, an inhibitor of H3K27 methylation may be administered in conjunction with MED13617. In some instances, an inhibitor of H3K27 methylation may be administered in conjunction with an antineoplastic agent. In some instances, an inhibitor of H3K27 methylation may be administered in conjunction with an agent targeting CSF-1R (also known as M-CSFR or CD115). In some instances, an inhibitor of H3K27 methylation may be administered in conjunction with anti-CSF-1R (also known as IMC-CS4). In some instances, an inhibitor of H3K27 methylation may be administered in conjunction with an interferon, for example interferon alpha or interferon gamma. In some instances, an inhibitor of H3K27 methylation may be administered in conjunction with Roferon-A (also known as recombinant Interferon alpha-2a). In some instances, an inhibitor of H3K27 methylation may be administered in conjunction with GM-CSF (also known as recombinant human granulocyte macrophage colony stimulating factor, rhu GM-CSF, sargramostim, or LEUKINE®). In some instances, an inhibitor of H3K27 methylation may be administered in conjunction with IL-2 (also known as aldesleukin or PROLEUKIN®). In some instances, an inhibitor of H3K27 methylation may be administered in conjunction with IL-12. In some instances, an inhibitor of H3K27 methylation may be administered in conjunction with an antibody targeting CD20. In some instances, the antibody targeting CD20 is obinutuzumab (also known as GA101 or GAZYVA®) or rituximab. In some instances, an inhibitor of H3K27 methylation may be administered in conjunction with an antibody targeting GITR. In some instances, the antibody targeting GITR is TRX518.

In some instances, an inhibitor of H3K27 methylation may be administered in conjunction with a cancer vaccine. In some instances, the cancer vaccine is a peptide cancer vaccine, which in some instances is a personalized peptide vaccine. In some instances the peptide cancer vaccine is a multivalent long peptide, a multi-peptide, a peptide cocktail, a hybrid peptide, or a peptide-pulsed dendritic cell vaccine (see, e.g., Yamada et al., Cancer Sci. 104:14-21, 2013). In some instances, an inhibitor of H3K27 methylation may be administered in conjunction with an adjuvant. In some instances, an inhibitor of H3K27 methylation may be administered in conjunction with a treatment including a TLR agonist, e.g., Poly-ICLC (also known as HILTONOL®), LPS, MPL, or CpG ODN. In some instances, an inhibitor of H3K27 methylation may be administered in conjunction with tumor necrosis factor (TNF) alpha. In some instances, an inhibitor of H3K27 methylation may be administered in conjunction with IL-1, e.g., IL-1β. In some instances, an inhibitor of H3K27 methylation may be administered in conjunction with HMGB1. In some instances, an inhibitor of H3K27 methylation may be administered in conjunction with an IL-10 antagonist. In some instances, an inhibitor of H3K27 methylation may be administered in conjunction with an IL-4 antagonist. In some instances, an inhibitor of H3K27 methylation may be administered in conjunction with an IL-13 antagonist. In some instances, an inhibitor of H3K27 methylation may be administered in conjunction with an HVEM antagonist. In some instances, an inhibitor of H3K27 methylation may be administered in conjunction with an ICOS agonist, e.g., by administration of ICOS-L, or an agonistic antibody directed against ICOS. In some instances, an inhibitor of H3K27 methylation may be administered in conjunction with a treatment targeting CX3CL1. In some instances, an inhibitor of H3K27 methylation may be administered in conjunction with a treatment targeting CXCL9. In some instances, an inhibitor of H3K27 methylation may be administered in conjunction with a treatment targeting CXCL10. In some instances, an inhibitor of H3K27 methylation may be administered in conjunction with a treatment targeting CCLS. In some instances, an inhibitor of H3K27 methylation may be administered in conjunction with an LFA-1 or ICAM1 agonist. In some instances, an inhibitor of H3K27 methylation may be administered in conjunction with a Selectin agonist.

In general, for the prevention or treatment of disease, the appropriate dosage of the additional therapeutic agent will depend on the type of disease to be treated, the type of antibody, the severity and course of the disease, whether the one or more inhibitors of H3K27 methylation and/or additional agent are administered for preventive or therapeutic purposes, previous therapy, the patient's clinical history and response to the inhibitor of H3K27 methylation and additional agent, and the discretion of the attending physician. The inhibitor of H3K27 methylation and additional agent are suitably administered to the patient at one time or over a series of treatments. The inhibitor of H3K27 methylation is typically administered as set forth above. Depending on the type and severity of the disease, about 20 mg/m² to 600 mg/m² of the additional agent is an initial candidate dosage for administration to the patient, whether, for example, by one or more separate administrations, or by continuous infusion. One typical daily dosage might range from about or about 20 mg/m², 85 mg/m², 90 mg/m², 125 mg/m², 200 mg/m², 400 mg/m², 500 mg/m² or more, depending on the factors mentioned above. For repeated administrations over several days or longer, depending on the condition, the treatment is sustained until a desired suppression of disease symptoms occurs. Thus, one or more doses of about 20 mg/m², 85 mg/m², 90 mg/m², 125 mg/m², 200 mg/m², 400 mg/m², 500 mg/m², 600 mg/m² (or any combination thereof) may be administered to the patient. Such doses may be administered intermittently, e.g., every week or every two, three weeks, four, five, or six (e.g., such that the patient receives from about two to about twenty, e.g., about six doses of the additional agent). An initial higher loading dose, followed by one or more lower doses may be administered. However, other dosage regimens may be useful. The progress of this therapy is easily monitored by conventional techniques and assays.

In one embodiment, the subject has never been previously administered any drug(s) to treat cancer. In another embodiment, the subject or patient have been previously administered one or more medicaments(s) to treat cancer. In a further embodiment, the subject or patient was not responsive to one or more of the medicaments that had been previously administered. Such drugs to which the subject may be non-responsive include, for example, anti-neoplastic agents, chemotherapeutic agents, cytotoxic agents, and/or growth inhibitory agents.

### IV. Compositions

In one aspect, the invention is based, in part, on the discovery that combinations including inhibitors of H3K27 methylation (e.g., H3K27me3 inhibitors, e.g., EZH2 inhibitors) are useful for treating patients suffering from cancer, wherein the cancer is associated with a decreased expression of SMARCA2 and/or an increased occupancy of H3K27 (e.g., H3K27me3) at a SMARCA2 promoter, relative to a reference level.

In certain embodiments, provided is a composition comprising one or more inhibitors of H3K27 methylation (e.g., an H3K27me3 inhibitor, e.g., an EZH2 inhibitor, e.g., EPZ-6438, CPI-169, EPZ005687, GSK-126, GSK343, or GSK503) for use in a method of treating a patient suffering from a cancer (e.g., rhabdoid cancer (e.g., malignant rhabdoid cancer (e.g., malignant rhabdoid brain cancer or malignant rhabdoid renal cancer)), ovarian cancer (e.g., ovarian clear cell carcinoma, or a small cell carcinoma of the ovary, e.g., a small cell carcinoma of the ovary, hypercalcemic type), lung cancer, gastric cancer, bladder cancer, breast cancer, skin cancer, colorectal cancer, stomach cancer, lymphoid cancer, cervical cancer, peritoneal cancer, pancreatic cancer, glioblastoma, liver cancer, bladder cancer, colon cancer, endometrial cancer, uterine cancer, renal cancer, prostate cancer, thyroid cancer, and head and neck cancer), wherein a sample obtained from the patient has been determined to have a decreased expression level of SMARCA2 in a sample as compared to a reference expression level.

In other embodiments, provided is a composition comprising one or more inhibitors of H3K27 methylation (e.g., an H3K27me3 inhibitor, e.g., an EZH2 inhibitor, e.g., EPZ-6438, CPI-169, EPZ005687, GSK-126, GSK343, or GSK503) for use in a method of treating a patient suffering from a cancer (e.g., rhabdoid cancer (e.g., malignant rhabdoid cancer, e.g., malignant rhabdoid brain cancer or malignant rhabdoid renal cancer), ovarian cancer (e.g., ovarian clear cell carcinoma, or a small cell carcinoma of the ovary, e.g., a small cell carcinoma of the ovary, hypercalcemic type), lung cancer, gastric cancer, bladder cancer, breast cancer, skin cancer, colorectal cancer, stomach cancer, lymphoid cancer, cervical cancer, peritoneal cancer, pancreatic cancer, glioblastoma, liver cancer, bladder cancer, colon cancer, endometrial cancer, uterine cancer, renal cancer, prostate cancer, thyroid cancer, and head and neck cancer), wherein a sample obtained from the patient has been determined to have an increased occupancy level of H3K27 (e.g., H3K27me3) at a SMARCA2 promoter in a sample as compared to a reference occupancy level.

### V. Diagnostic Kits

Provided herein are diagnostic kits including one or more reagents (e.g., polypeptides or polynucleotides) for determining the presence of a biomarker (e.g., SMARCA2 repression) in a sample from an individual or patient with a disease or disorder (e.g., a proliferative cell disorder (e.g., cancer ((e.g., rhabdoid cancer (e.g., malignant rhabdoid cancer, e.g., malignant rhabdoid brain cancer or malignant rhabdoid renal cancer), ovarian cancer (e.g., ovarian clear cell carcinoma, or a small cell carcinoma of the ovary, e.g., a small cell carcinoma of the ovary, hypercalcemic type), lung cancer, gastric cancer, bladder cancer, breast cancer, skin cancer, colorectal cancer, stomach cancer, lymphoid cancer, cervical cancer, peritoneal cancer, pancreatic cancer, glioblastoma, liver cancer, bladder cancer, colon cancer, endometrial cancer, uterine cancer, renal cancer, prostate cancer, thyroid cancer, and head and neck cancer))). In some instances, a decreased level of expression of the biomarker in the sample identifies a patient with a higher likelihood of benefiting from treatment with an inhibitor of H3K27 methylation. In some instances, the decreased presence of the biomarker in the sample, relative to a reference level, indicates a higher likelihood of efficacy when the individual is treated with an inhibitor of H3K27 methylation. Optionally, the kit may further include instructions to use the kit to identify a patient with a higher likelihood of benefiting from treatment with an inhibitor of H3K27 methylation. In another instance, the kit may further include instructions to use the kit to select a medicament (e.g., a medicament including an inhibitor of H3K27 methylation, e.g., an EZH2 inhibitor, e.g., EZP-6438) for treating the disease or disorder (e.g., cancer) if the individual expresses a decreased level of the biomarker in the sample, relative to a reference expression level.

In another embodiment, diagnostic kits may include one or more reagents (e.g., reagents capable of determining the occupancy level of H3K27 (e.g., H3K27me3) at a SMARCA2 promoter in a sample, e.g., ChIP-seq or ChIP-PCR reagents) for determining the presence of a biomarker (e.g., H3K27 at a SMARCA2 promoter) in a sample from an individual or patient with a disease or disorder (e.g., a proliferative cell disorder (e.g., cancer ((e.g., rhabdoid cancer (e.g., malignant rhabdoid cancer, e.g., malignant rhabdoid brain cancer or malignant rhabdoid renal cancer), ovarian cancer (e.g., ovarian clear cell carcinoma, or a small cell carcinoma of the ovary, e.g., a small cell carcinoma of the ovary, hypercalcemic type), lung cancer, gastric cancer, bladder cancer, breast cancer, skin cancer, colorectal cancer, stomach cancer, lymphoid cancer, cervical cancer, peritoneal cancer, pancreatic cancer, glioblastoma, liver cancer, bladder cancer, colon cancer, endometrial cancer, uterine cancer, renal cancer, prostate cancer, thyroid cancer, and head and neck cancer))). In some instances, the presence or level of occupancy of the biomarker in the sample identifies a patient with a higher likelihood of benefiting from treatment with an inhibitor of H3K27 methylation. In some instances, an increased level of occupancy of the biomarker in the sample, relative to a reference level of occupancy, indicates a higher likelihood of efficacy when the individual is treated with an inhibitor of H3K27 methylation. Optionally, the kit may further include instructions to use the kit to identify a patient with a higher likelihood of benefiting from treatment with an inhibitor of H3K27 methylation. In another instance, the kit may further include instructions to use the kit to select a medicament (e.g., a medicament including an inhibitor of H3K27 methylation, e.g., an EZH2 inhibitor, e.g., EZP-6438) for treating the disease or disorder (e.g., cancer) if the individual expresses an increased level of occupancy of the biomarker in the sample, relative to a reference level.

Any embodiment of a kit described herein may further include one or more reagents (e.g., polypeptides or polynucleotides) for identifying a mutation in one or more genes encoding a nucleosome remodeling protein (e.g., a SWI/SNF complex protein, e.g., a gene encoding BRG1, SNF5, INI1, or BAF, e.g., SMARCA4, SMARCB1, SMARCC1, SMARCC2, ARID1A, ARID2, and PBRM1) in a sample from an individual or patient with a disease or disorder (e.g., a proliferative cell disorder (e.g., cancer ((e.g., rhabdoid cancer (e.g., malignant rhabdoid cancer, e.g., malignant rhabdoid brain cancer or malignant rhabdoid renal cancer), ovarian cancer (e.g., ovarian clear cell carcinoma, or a small cell carcinoma of the ovary, e.g., a small cell carcinoma of the ovary, hypercalcemic type), lung cancer, gastric cancer, bladder cancer, breast cancer, skin cancer, colorectal cancer, stomach cancer, lymphoid cancer, cervical cancer, peritoneal cancer, pancreatic cancer, glioblastoma, liver cancer, bladder cancer, colon cancer, endometrial cancer, uterine cancer, renal cancer, prostate cancer, thyroid cancer, and head and neck cancer))). In some instances, the presence of a mutation in one or more genes encoding a nucleosome remodeling protein identifies a patient with a higher likelihood of benefiting from treatment with an inhibitor of H3K27 methylation. In some instances, the presence of a mutation in one or more genes encoding a nucleosome remodeling protein identifies a patient with a higher likelihood of having a repressed expression level of SMARCA2 (e.g., a decreased expression level relative to a reference expression level) or an increased occupancy of H3K27 (e.g., H3K27me3) at a SMARCA2 promoter, relative to a reference occupancy level. In some embodiments, the kit may further include instructions to use the kit to test for SMARCA2 repression and/or H3K27 occupancy at a SMARCA2 promoter in a sample if the sample has a mutation in one or more genes encoding a nucleosome remodeling protein (e.g., a SWI/SNF complex protein, e.g., a gene encoding BRG1, SNF5, INI1, or BAF, e.g., SMARCA4, SMARCB1, SMARCC1, SMARCC2, ARID1A, ARID2, and PBRM1). Optionally, the kit may further include instructions to use the kit to identify a patient with a higher likelihood of benefiting from treatment with an inhibitor of H3K27 methylation. In another instance, the kit may further include instructions to use the kit to select a medicament (e.g., a medicament including an inhibitor of H3K27 methylation, e.g., an EZH2 inhibitor, e.g., EZP-6438) for treating the disease or disorder (e.g., cancer) according to the results of the one or more tests.

### EXAMPLES

The following examples are provided to illustrate, but not to limit the presently claimed invention.

### Example 1. Materials and Methods

### Cell Lines and Culture

All cells were maintained in RPM11640 supplemented with 10% Fetal Bovine Serum (FBS) and GlutaMAX under 5% CO₂ at 37° C. Stable Cas9 expressing lines were generated through infection with lentivirus expressing Cas9 (pLenti6.3) followed by selection with blasticidin. For generation of EZH2-knockout cell lines, guide RNAs targeting EZH2 (targeting sequences: gEZH2-#4, AAGACCCCACCAAAACGTCCAGG (SEQ ID NO: 25); gEZH2-#5, TGGGGTCTTTATCCGCTCAGCGG (SEQ ID NO: 26)) and controls (gLuc-#1, gLuc-#2) were cloned into the pLKO.1 vector. Lentiviral packaging 293T cells were plated 48 hours prior to transfection with a 1:2.3:0.2 molar ratio DNA mix of 5 ug of pLKO.1-puro gRNA plasmid, delta8.9 and VSVG. Transfections were carried out with lipofectamine 2000 (2 p1/μg DNA, Thermo Fisher). Virus was harvested 72 hour post-transfection. Target cells were infected with a 1/10 dilution of the media collected from the 293T cells. Infected target cells were selected with a toxic concentration of puromycin after 72 hours post-transfection.

### Clonogenic Assay

1,800-5,000 cells were plated in each 6-well plate, according to the doubling time. 24 hours after plating, the medium was removed and replaced with medium containing EPZ-6438 at different concentrations. Fresh medium with EPZ-6438 was replaced every 3 to 4 days until control cells reached confluence to stop culture. For studies evaluating the effect of EZH2 knockout, cells were plated 7 days following puromycin selection. The medium was removed, and cells were washed with PBS and stained with 0.5% crystal violet for 20 minutes at room temperature. Dye was removed, cell monolayers were washed with water, and the plate was washed and photographed.

### Evaluation of Apoptosis and Senescence

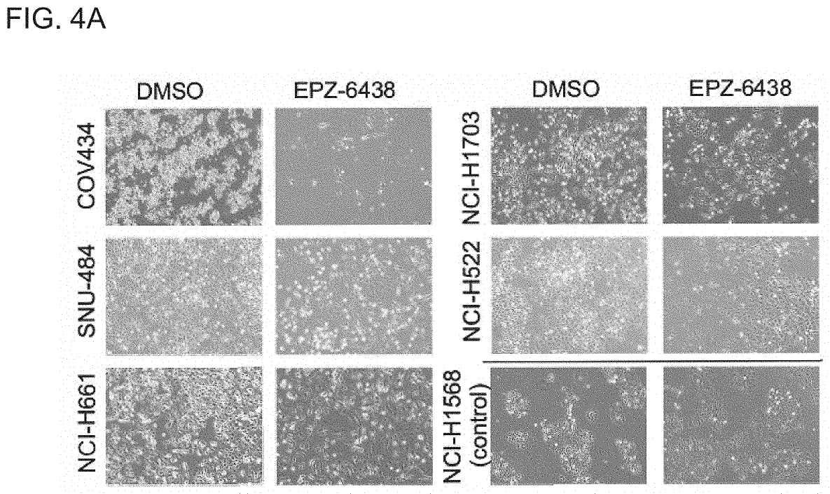

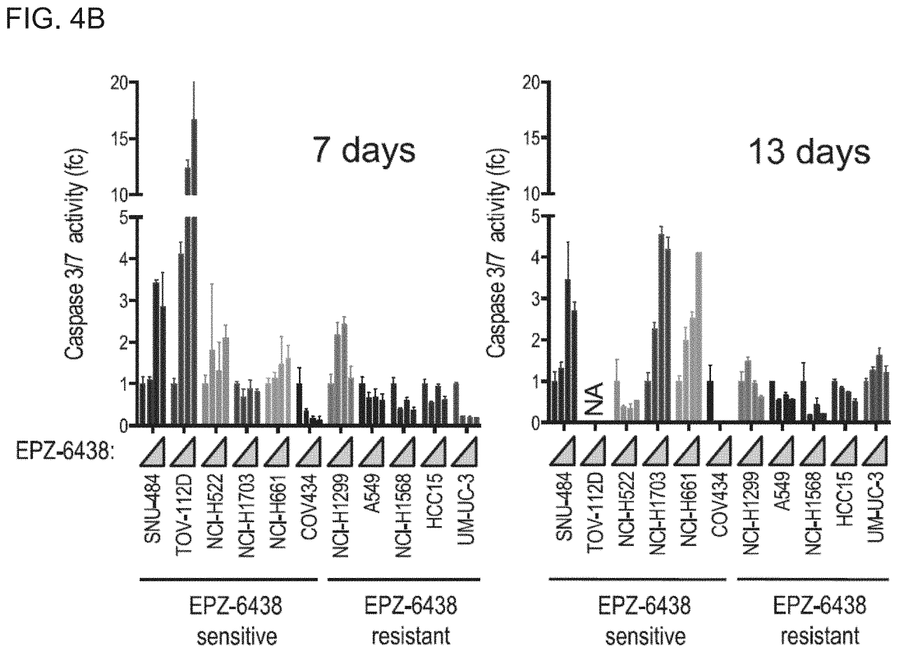

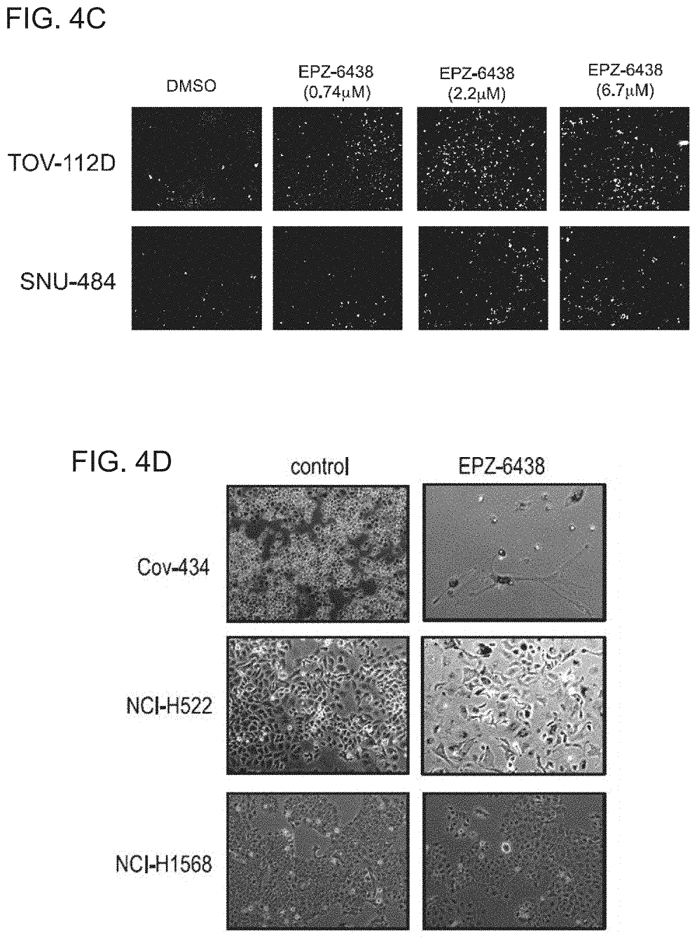

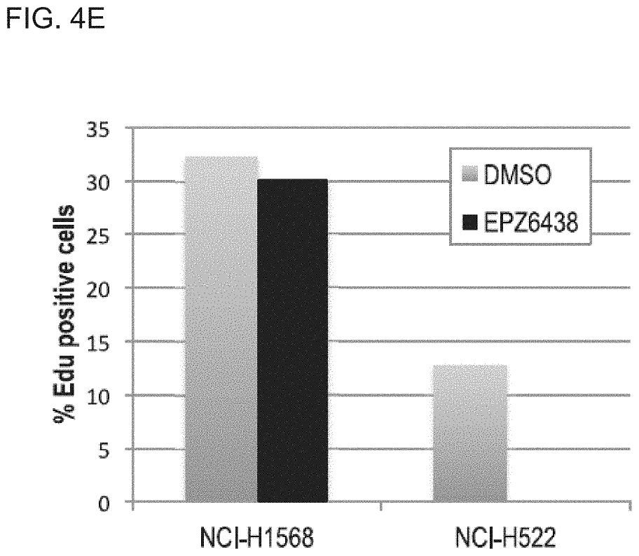

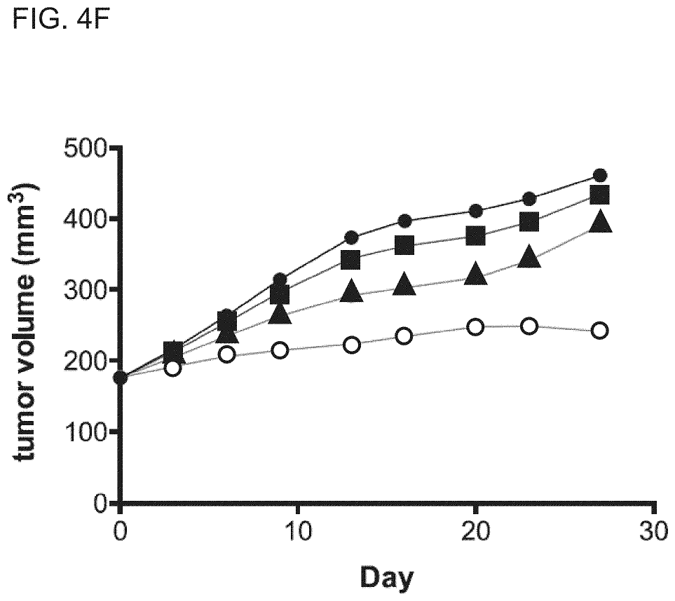

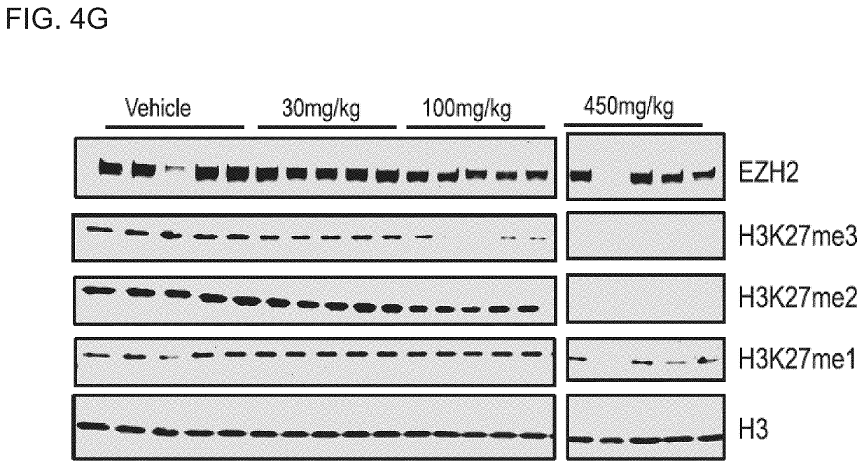

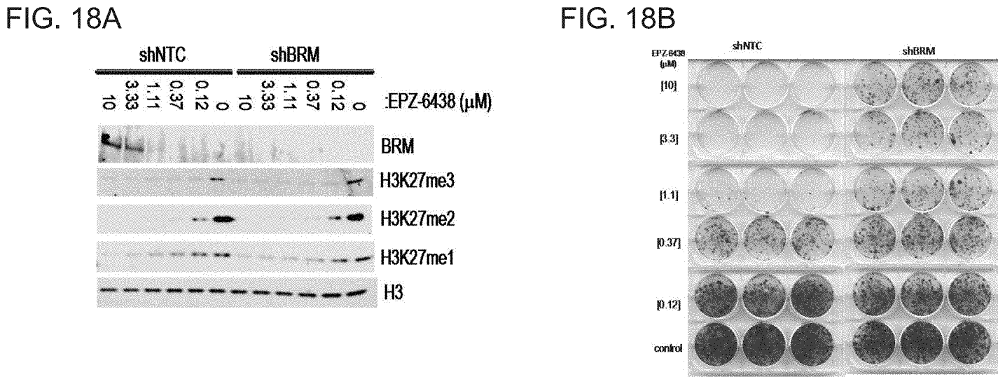

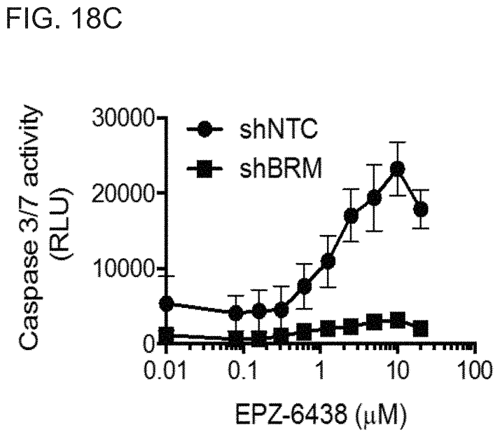

Apoptosis was monitored through (a) live cell imaging analysis using the Incucyte Caspase-3/7 Apoptosis Assay (Essen Biosciences, Cat. No. 4440) or through a static time point assessment using the Caspase-Glo 3/7 Assay (Promega, G8090). For Incucyte-based assays (FIG. 4B), 300-600 cells (based on doubling time) were plated in 96-well plates, and at 24 hours, media was replaced with EPZ-6438-containing media at the indicated concentrations and Caspase 3/7 reagent (Essen Bioscience). Fresh media containing EPZ-6438 and Caspase 3/7 reagent was replaced every 3 to 4 days. Phase contrast and fluorescent images were collected every 3 hours, and the number of fluorescent objects were counted and analyzed according to the Incucyte protocol. Data are presented as Caspase 3/7 fluorescent counts normalized to DMSO control at the indicated time points. For determination of apoptosis by Caspase-Glo (FIG. 18C, 19G), TOV112D cells were plated at 500 cells per well in a 96 well plate and treated with the indicated concentrations of EPZ-6438 for 6 days. Caspase 3/7 activity was measured according to the manufacturer's instructions, and results were normalized to signal in DMSO control wells, when indicated. To evaluate senescence induction, cells were stained for β-galactosidase activity using the Senescence Cells Histochemical Staining Kit (Sigma), according to the manufacturers instructions.

### Subcellular Fractionation.

To determine the relative subcellular distribution of proteins, 3×10⁶ cells were resuspended in 200 μl of Buffer A containing 10 mM HEPES, [pH 7.9], 10 mM KCl, 1.5 mM MgCl₂, 0.34 M sucrose, 10% glycerol, 1 mM DTT, and protease phosphatase inhibitors. Triton X-100 from a 10% stock was added to a final concentration of 0.1% and immediately mixed. The lysate was incubated on ice for 5 minutes and then spun at 1300 g for 4 minutes at 4° C. The supernatant containing the cytosolic fraction was carefully removed, and the nuclei pellet was washed once with Buffer A without TritonX-100 and then spun down at 1300 g for 4 minutes at 4° C. The pellet was resuspended in Buffer B (3 mM EDTA, 0.2 mM EGTA, 1 mM DTT, protease phosphatase inhibitors) and incubated on ice for 30 minutes prior to centrifugation at 1700 g for 4 minutes at 4° C. The supernatant containing the soluble nuclear protein was removed and the chromatin pellet was further washed with 200 μl Buffer B and centrifuged at 1700 g for 4 minutes at 4° C. The pellet was resuspended in Buffer C (50 mM Tris-HCl, [pH 7.4], 0.5 M NaCl, 1% TritonX-100 and 0.1% SDS) and sonicated for 30 rounds of 20 seconds on and 30 seconds off prior to analysis by SDS-PAGE and Western blotting.

### Western Blot

For studies evaluating EPZ-6438 effects, cells were treated with various doses of EPZ-6438 for 6 days. On day 3, fresh medium containing EPZ-6438 was introduced. Cell pellets were lysed in RIPA buffer containing 1M NaCl and homogenized for 3 minutes at speed 10 (NextAdvance, Bullet Blender® 24). 12 μg or 18 μg protein was dissolved in 4-12% bis-Tris or 3-8% Tris-acetate gel and transferred to polyvinylidend difluoride (PVDF) membranes. Membranes were incubated overnight with primary antibodies as indicated in Table 1, below. IRDYE® secondary antibodies were used for detection by an Odyssey Imager (LI-COR).

TABLE 1
Antibodies used for protein detection

| Protein | Antibody Clone |
|---|---|
| EZH2 | CST 5246 |
| SUZ12 | active motif 39357 |
| H3K27me3 | CST 9733 |
| H3K27me2 | CST 9728 |
| H3K27me1 | active motif 61015 |
| H3K27ac | active motif 39685 |
| H3K36me2 | active motif 61019 |
| Total H3 | CST 3638 |
| BRG1 (SMARCA4) | sc-17796 |
| BRM (SMARCA2) | CST 11966 |
| SMARCA4 | sc-17796 |
| SMARCA2 | CST 11966 |
| SMARCC1 (155) | sc-9746 |
| SMARCC2 (BAF170) | A301-039A |
| SMARCB1 (SNF5) | CST8745 |
| SMARCE1 (BAF57) | A300-810A |
| SMARCD1 (BAF60A) | A301-595A |
| ARID1A | CST12354 |
| ACTL6A (BAF53a) | A301-391 |
| ARID2 | A302-230A |
| PBRM1 | A301-591A |
| Actin | CST 4970 |

### Immunoprecipitation

For coimmunoprecipitation, nuclear pellets of 8×10⁶ cells were lysed in 100 μl nuclear lysis buffer (50 mM Hepes (pH 7.8), 3 mM MgCl2, 25% glycerol, 0.5% Nonidet P-40, 0.42 M NaCl, 300 mM NaCl, 1 mM DTT, 0.1 mM PMSF, DNase 5 U/μl, Benzonase 5 U/μl, and protease and phosphatase inhibitors). The suspension was incubated at 37° C. for 10 minutes and the nuclease reaction was stopped with 2 μl of 0.5 M EDTA. The nuclear fraction was collected after centrifugation (14000 g) for 10 min. Lysate was precleared using 30 μL Oynabeads Protein G (Life Technologies) for 60 min at 4° C. with gentle rotation. A fraction (10%) of the lysate was taken as an input control. The remaining lysate was incubated with 5 μg of primary anti-SMARCC1 IgG overnight prior to the addition of 50 μL Oynabeads Protein G and incubation for an additional 2 hours at 4° C. with gentle rotation. Immunopreciptiations were washed twice using low-salt coimmunoprecipitation wash buffer (20 mM Tris-HCl, pH 8.0, 150 mM NaCl, 1.5 mM MgCl₂, 0.5% Nonidet P-40, 0.2 mM EDTA) prior to the addition of 30 μL NuPAGE LDS sample buffer containing DTT (Bio-Rad) and heated at 95° C. for 5 minutes. Supernatants were immunoblotted for the indicated proteins.

### RNA Interference, CRISPR Gene Editing, and Inducible-Orf Expression.

Individual shRNAs targeting SMARCA2, CTSB, or controls were designed using the DSIR algorithm and cloned into a modified pLKO lentiviral vector using the miR-3G hairpin expression context, as described in Watanabe et al., RNA Biol. 13(1):25-33 (2016). The following shRNA sequences were used: shNTC (5′-AACCACGTGAGGCATCCAGGC-3′; SEQ ID NO: 29), shSMARCA2 (5′-TCGTCGAGCAATCATTTGGTT-3′; SEQ ID NO: 30), shCTSB-1 (5′-TTCGATTCCACAGTGATCCTG-3′; SEQ ID NO: 31), shCTSB-2 (5′-TTGTAGGTCGGGCTGTAGCCA-3′; SEQ ID NO: 32), and shCTSB-3 (5′-TAGTTGACCAGCTCATCCGAC-3′; SEQ ID NO: 33).

Guide RNAs targeting EZH2 or LacZ controls were designed using the MIT algorithm (Crispr Design Tool. Zhang Lab, M I T, 2015. Web) and cloned into pLKO lentiviral vectors for stable lentiviral infection. The following guide RNA sequences were used: gluc-1 (5′-GCCGGCGCCATTCTATCCGC-3′; SEQ ID NO: 34), gluc-2 (5′-GGCATGCGAGAATCTCACGC-3′; SEQ ID NO: 35), gEZH2-4 (5′-AAGACCCCACCAAAACGTCC-3′; SEQ ID NO: 36), and gEZH2-5 (5′-TGGGGTCTTTATCCGCTCAG-3′; SEQ ID NO: 37). Paired guide RNAs (5′-GACAGCTCTACTGTATGCG-3′; SEQ ID NO: 38 and 5′-CTCTCACCAAGACGCCGAG-3′; SEQ ID NO: 39) targeting SMARCA2 were cloned into the pUC57_AIO_U6H1_EF1_Cas9_eGFP vector context for transfection to co-express the guide RNAs, Cas9 and an eGFP reporter. Sequence-verified Cas9 was cloned into the lentiviral vector pLenti6.3 for stable expression of Cas9 in cancer cell lines. SMARCA2 (NM_003070.4) and SMARCA4 (NM_003072.3) open reading frames were cloned into the doxycycline inducible vector, pinducer20.

To generate lentiviral particles, 293T cells were transfected with delta8.9 packaging plasmid, VSVG-envelope plasmid and respective pLKO vectors using Lipofectamine 2000 (Invitrogen). Media containing lentiviral particles was collected 48 hours after transfection, filtered through a 0.45-picometer filter, and used to transduce the respective cancer cell lines in the presence of 8 mg/ml polybrene. A spin-infection protocol was applied using 6-well plates at 1800 rpm for 45 minutes (Allegen X-12R Centrifuge, Beckman Coulter), followed by incubation at 37° C. for three days prior to addition of puromycin (1-1.5 μg/ml) or G418 (500 μg/ml). For generation of TOV112D SMARCA2 knock-out clones, cells were transfected with the pUC57_AIO_U6H1_EF1_Cas9_eGFP vector using Lipofectamine 2000 (Invitrogen). Three days following transfection, cells were GFP-sorted and single cell-cloned. Clones were sequenced to confirm SMARCA2 gene disruption.

### RNA-Seq

Total RNA was extracted using Qiagen RNeasy Plus Mini kit, according to the manufacturers protocol. Quality control of samples was performed to determine RNA quantity and quality prior to their processing by RNA-seq. The concentration of RNA samples was determined using NanoDrop 8000 (Thermo Scientific) and the integrity of RNA was determined by Fragment Analyzer (Advanced Analytical Technologies). 0.5 μg of total RNA was used as an input material for library preparation using TruSeq RNA Sample Preparation Kit v2 (Illumina). Size of the libraries was confirmed using 2200 TapeStation and High Sensitivity D1K screen tape (Agilent Technologies), and their concentration was determined by qPCR based method using Library quantification kit (KAPA). The libraries were multiplexed and sequenced on Illumina HiSeq2500 (Illumina) to generate 30M of single end 50 base pair reads.

The fastq sequence files for all RNA-seq samples were filtered for read quality (keeping reads where at least 70% of the cycles had Phred scores 23) and ribosomal RNA contamination. The remaining reads were then aligned to the human reference genome (GRCh38) using the GSNAP alignment tool, as described in Wu and Nacu, Bioinformatics. 26(7):873-881 (2010). Alignments were produced using the following GSNAP parameters: “-M 2 -n 10 -B 2 -i 1 -N 1 -w 200000 -E 1 -pairmax-ma=200000 -clip-overlap”. These steps, and the downstream processing of the resulting alignments to obtain read counts per gene (over coding exons of RefSeq gene models), were implemented in the Bioconductor package, HTSeqGenie (v 4.2.0). Only uniquely mapped reads were used for downstream analysis. All experiments were performed and sequenced in triplicate, with the exception of the panel of untreated SMARCA4-mutant, EPZ-6438-sensitive, and EPZ-6438-resistant cell lines, in which untreated lines were sequenced as singletons.

### Gene Expression Level Estimation and Identification of Differently Expressed Genes

For the following analyses, only genes for which expression levels were reliably estimated in multiple samples were considered (more than fifteen aligned reads observed in at least four samples). Gene expression estimates were generated using the voom/limma analytical framework (version 3.28.17), adjusting the observed library sizes with the calcNormFactors( ) function, as described in Law et al., Genome Biol. 15(2):R29 (2014).

For each gene, differential expression was quantified in the framework of a precision-weighted linear model using the expression estimates and weights returned by voom. This approach was used to identify genes with significantly different expression levels between cell lines sensitive and resistant to EPZ-6438; between primary or shRNA-expressing TOV-112 cells that have or have not been treated with EPZ-6438; and between TOV-112 cells that do or do not express SMARCA2 or SMARCA4 constructs.

When estimating the effect of the shRNA knockdown, nonspecific effects of the shRNA construct were controlled for by fitting the following linear model to each gene:

yᵢⱼₖ=β+ηᵢ+ϕⱼ+ηϕᵢⱼ+ϵᵢⱼₖ

In this model all coefficients are fixed effects. Let yᵢⱼₖ represent the observed expression level of a gene expressing shRNA construct i in treatment condition j and experimental replicate k. β represents the intercept, i is a fixed effect capturing the shRNA that is expressed (shSMARCA2 or non-targeting control), ø is a fixed effect capturing the effect of EPZ-6438, ηϕ is an interaction effect capturing the effect of the drug in the cells where the shRNA hairpin specifically targets SMARCA2, and E represents the residual error, assumed to be normally distributed with variance σ². To determine the effect of the shRNA knockdown, the following hypotheses were compared:

H₀:β≠0,ηᵢ≠0,ϕⱼ≠0,ηϕᵢⱼ=0

H₁:β≠0,ηᵢ≠0,ϕⱼ≠0,ηϕᵢⱼ=0

Significance was assigned to each gene's observed expression differences on the basis of the moderated t-statistics generated after empirical Bayes variance shrinkage to generate p-values. These p-values were then corrected for multiple testing using the Benjamini-Hochberg approach, as described in Benjamini and Hochberg, Genome Biol. 15(2):R29 (2014). Genes with a corrected p-value less than 0.05 and a log 2 change in expression level greater than 1 were defined as differentially expressed.

### Taqman Gene Expression Assay

Cells were treated with 5 μM EPZ-6438 for 6 days or 10 days. Fresh media containing 5 μM EPZ-6438 were replaced every 3-4 days. Cells were harvested at day 6 or day 10. RNA was prepared by RNeasy Plus mini kits (QIAGEN). Gene expression level was detected by SMARCA2 probe (Hs01030846_m1) and Taqman One-Step RT-PCR Master Mix Reagents kit (ThermoFisher Scientific). Analysis was performed using 7900HT SDS (ThermoFisher Scientific). Expression levels are presented relative to the housekeeping gene, GAPDH (2^{-ΔCt}).

### ChIP-Seq

Cells were fixed with 1% formaldehyde for 10 minutes at room temperature. Chromatin was isolated by the addition of a standard lysis buffer containing 600 mM NaCl. DNA was sheared by sonication to 300 to 500-bp size fragments. Chromatin was immunoprecipitated with anti-H3K27me3 antibody (Millipore 07-449) in the presence of 0.4 μg H2Av antibody (Active motif 39715) and 750 ng of sonicated Drosophila chromatin. Illumina sequencing libraries were prepared from the ChIP and input DNAs. The resulting DNA libraries were quantified and sequenced as 150-bp paired-end reads using Illumina's HiSeq 2500. Fragments had average lengths of about 500 bp.

### ChIP-PCR

For each sample, 10×10⁶ cells were harvested and washed with 1×PBS. Cells were fixed and sheared following the instructions provided with the truChlP Chromatin Shearing Reagent Kit (Covaris). Cells were fixed for 5 minutes with 1% formaldehyde and then quenched with quenching buffer for 5 minutes. Cells were then washed with cold 2×PBS. Nuclei were isolated and sheared using the Covaris AFA Focused-ultrasonicator for 20 minutes. The IP was conducted with 500 μg sheared chromatin and 10 μg anti-H3K27me3 (Active Motif cat #39155) or anti-Rabbit IgG. The Magna ChIP kit (Millipore) was used for IP. For each IP, a 50 μl mixture of Dynabeads protein A and G (50/50 mix) was incubated with primary antibody for 3 hours. The beads were added to 500 μg of sheared chromatin. The beads and antibodies were incubated overnight at 4° C. The beads were then washed with the following wash buffers: low salt, high salt, LiCI wash buffer, and TE Buffer. DNA was extracted from the beads in ChIP elution buffer with protease K at 64° C. with shaking overnight. DNA was then purified using the QIAquick PCR Purification Kit (Qiagen). DNA was eluted with 30 μl of water. 1.0 μl of eluted DNA was used for each SYBR green PCR reaction. SMARCA2 was amplified using the following primers: forward, GTAGGCAGGCCTTTAGGCAA (SEQ ID NO: 27); reverse, GCCGGACATCCCGAACTTTA (SEQ ID NO: 28). Negative control primers to amplify regions devoid of H3K27me3 were purchased from Active Motif (Catalog No: 71001, 71002). The following PCR conditions were run: 50° C. for 2 minutes, 95° C. for 10 minutes, 40 cycles of 95° C. for 15 seconds and 57° C. for 1 minute.

### Methylcellulose Colony Formation

Wells of a 24 well plated were coated with 70 μl Matrigel Matrix (Corning) and allowed to congeal at room temperature. Cells (n=5,000) were plated on top of the basement matrix in 400 μl RPMI+10% FBS containing 2% Matrigel matrix. Cells were treated with the respective compounds in 400 μl media to replenish the old media. Colonies were imaged on a Zeiss Axio Observer A1 microscope 10 days after the start of experiment.

### Xenograft Studies

TOV-21G and NCI-H522 cells (American Type Culture Collection, Manassas, Va.) were cultured in vitro and harvested in HBSS:Matrigel (BD Biosciences; Franklin Lakes, N.J.) (1:1, v:v) for subcutaneous inoculation into female mice. TOV-21G cells were inoculated into Fox Chase SCIO® Beige mice (Charles River Laboratories, San Diego, Calif.). NCI-H522 cells were inoculated into BALB/c Nude mice (Vital River Laboratories, Beijing, China). Mice bearing established tumors were separated into groups of equally sized tumors (n=5, minimum) to receive escalating doses of EPZ-6438. EPZ-6438 was formulated once weekly in 0.5% sodium carboxymethylcellulose and 0.1% tween-80 at concentrations needed for target doses in a volume of 0.2 ml. All formulations were stored at 4° C., brought to room temperature, and mixed by vortex before oral administration by gavage twice daily from Day 1 until the end of the study. Tumor volumes were calculated from perpendicular length and width caliper measurements using the formula:

Tumor Volume (mm³)=0.5×(Length×Width²).

Plasma and tumor samples for pharmacodynamic analysis were collected from tumor-bearing mice on day 7, 3 hours following the last dose. Tumor tissue was lysed in RIPA buffer containing 1M NaCl and homogenized for 3 minutes at speed 10 (NEXTADVANCE, BULLET BLENDER® 24) prior to Western blotting.

A mixed modeling approach was used to analyze the repeated measurement of tumor volumes from the same animals over time. Cubic regression splines were used to fit a nonlinear profile to the time courses of log 2-transformed tumor volumes in each group. Fitting was done via a linear mixed-effects model, using the package “nlme” (version 3.1-97) in R version 2.13.0 (R Development Core Team 2008; R Foundation for Statistical Computing; Vienna, Austria). Fitted tumor volumes were plotted in the natural scale in Prism (version 5.0b for Mac) (GraphPad Software; La Jolla, Calif.).

### Example 2. Identification of EZP-6438 Resistant SMARCA4-Mutant Cell Lines Sensitive to EZH2 Inhibition

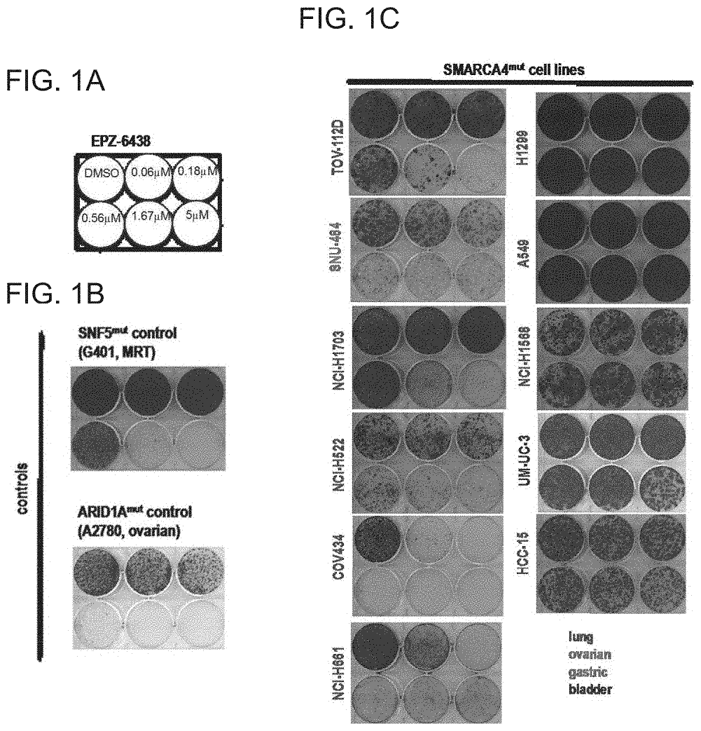

The EZH2-targeting histone methyltransferase inhibitor, EPZ-6438, was used as an inhibitor of H3K27 methylation to test the effects of H3K27me3 inhibition on colony formation across a panel of 11 SMARCA4-mutant cancer cell lines derived from different tumor types: ovarian cancer cells (TOV-112D and COV434), gastric cancer cells (SNU-484), lung cancer cells (NCI-H1703, NCI-H522, NCI-H661, H1299, A549, NCI-H1568, and HCC-15), and bladder cancer cells (UM-UC-3). A dose-dependent inhibition in colony formation was observed in a subset of these SMARCA4-mutant cells, which was independent of tissue derivation (FIGS. 1A and 1C). In addition, the degree of growth inhibition upon EPZ-6438 treatment was similar to that observed in models characterized by mutations in SMARCB1/SNF5 (G401) or ARID1A (A2780) (FIG. 1B). No activity was observed in a panel (n=8) of SWI/SNF wild-type models.

### Example 3. Assessment of EZH2 Inhibition Specificity

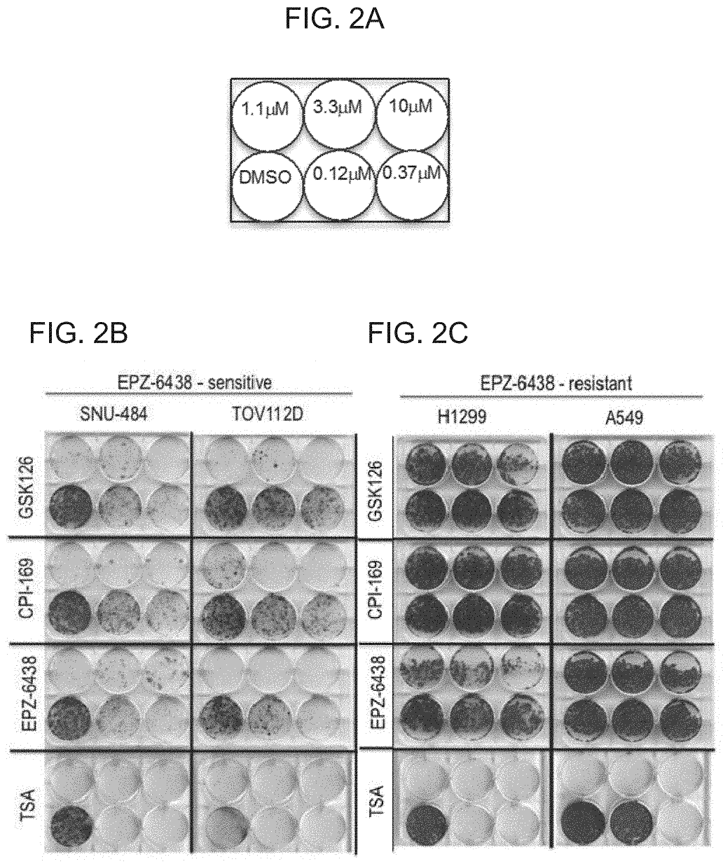

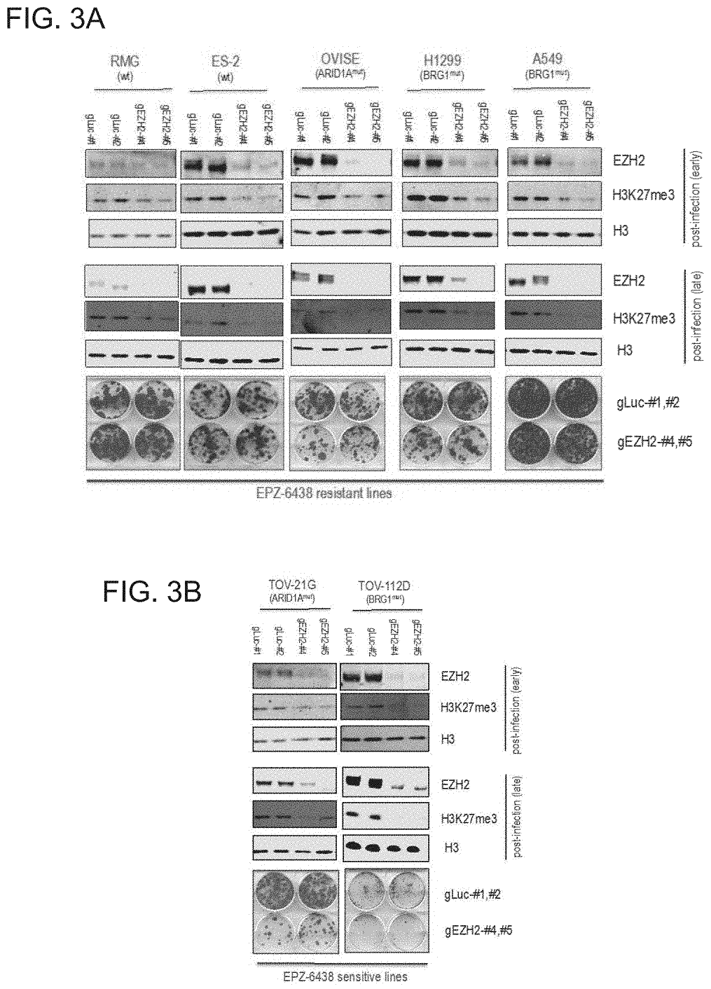

To determine if the effects of EPZ-6438 were specific to EZH2 inhibition, two additional EZH2 methyltransferase inhibitors, GSK-126 and CPI-169, were tested for effects on colony formation. As was observed with EPZ-6438, GSK-126 and CPI-169 inhibited colony formation in SMARCA4-mutant cells that were sensitive to EPZ-6438 in a dose-dependent manner, but had no effect on SMARCA4-mutant cells that were resistant to EPZ-6438 (FIGS. 2A-2C). In addition, genetic deletion of EZH2 through CRISPR resulted in an inhibition of colony formation in SMARCA4-mutant cells sensitive to EPZ-6438 (TOV-112D), but it had no effect on colony formation in EPZ-6438-resistant, SMARCA4-mutant cells (H1299 and A549; FIGS. 3A and 3B). Taken together, these data show that the effect of EPZ-6438 on colony formation in SMARCA4-mutant cells is on-target and dependent upon EZH2.

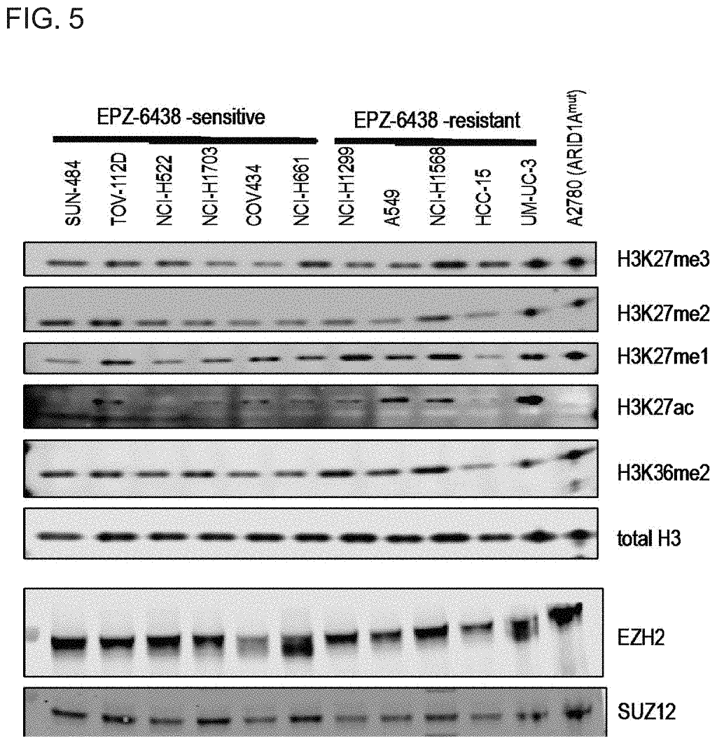

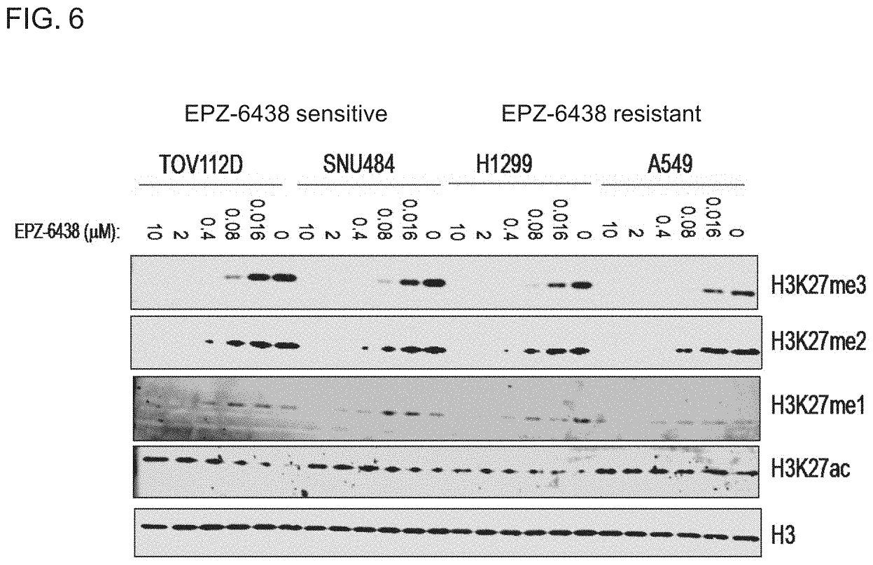

To determine if the differential sensitivity of SMARCA4-mutant cancer cells to EPZ-6438 is related to differential global PRC2 activity, levels of H3K27 methylation were examined. No apparent differences were observed in mono-, di-, or tri-methylated H3K27 amongst EPZ-6438-sensitive and EPZ-6438-resistant cell lines, nor were any differences in expression levels of the PRC2 components EZH2 or SUZ12, observed (FIG. 5). Furthermore, EPZ-6438 inhibited mono-, di-, and tri-methylated H3K27 to a similar extent amongst EPZ-6438-sensitive and EPZ-6438-resistant cell lines in a dose-dependent manner, indicating that the differential cellular activity was not due to differences in the ability of EPZ-6438 to inhibit EZH2 (FIG. 6).

EZH2 inhibition led to a heterogeneous phenotypic response. In contrast to resistant models, EPZ-6438-sensitive models consistently acquired pronounced morphologic changes after 21 days of treatment, characterized by cell flattening and enlargement (FIG. 4A). A strong apoptotic response was observed in TOV-112D cells following seven days of EPZ-6438 treatment, whereas several other models showed evidence for subpopulations of apoptotic cells following prolonged exposure with EPZ-6438 (FIGS. 4B and 4C). Increases in senescence-associated β-galactosidase expression were observed in some SMARCA4-mutant EPZ-6438-sensitive models. This was most notable in the COV434 and NCI-HS22 cell lines that lacked evidence for apoptosis (FIG. 4D). Additionally, subpopulations of β-galactosidase positive cells (e.g., NCI-H661 cells) exhibited evidence for apoptosis at later time points. The kinetics of senescence induction varied. For example, the COV434 model exhibiting homogenous expression of β-galactosidase by seven days of treatment with EPZ-6438, whereas homogenous β-galactosidase expression was not observed until a few weeks of EPZ-6438 treatment in NCI-HS22 cells, despite these cells remaining in a non-proliferative state, based on Edu incorporation (FIG. 4E). Treatment of SCID mice bearing NCI-HS22 cells grown as xenografts resulted in a dose-dependent inhibition of tumor growth following twice daily (BID) administration of EPZ-6438 (FIG. 4F), in which the strongest tumor growth inhibition (72% TGI) and reduction of H3K27me3 and H3K27me2 occurred in response to the 450 mg/kg BID dose (FIG. 4G).

### Example 4. Identification of SMARCA2 Repression as a Biomarker for Inhibitor of H3K27 Methylation Sensitivity

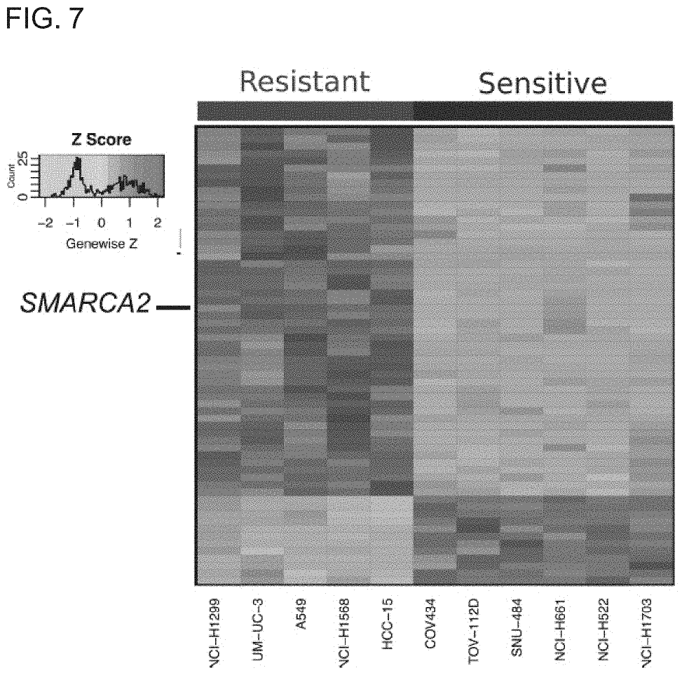

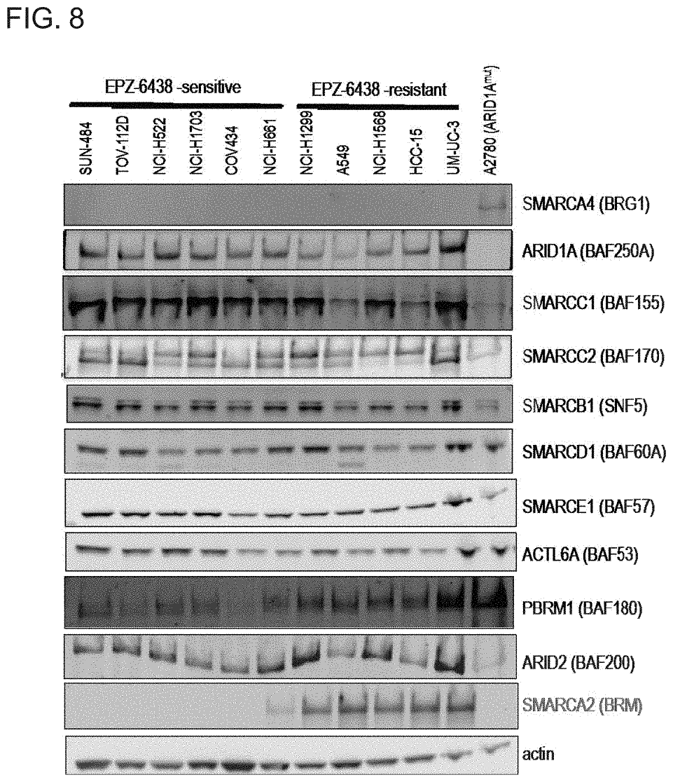

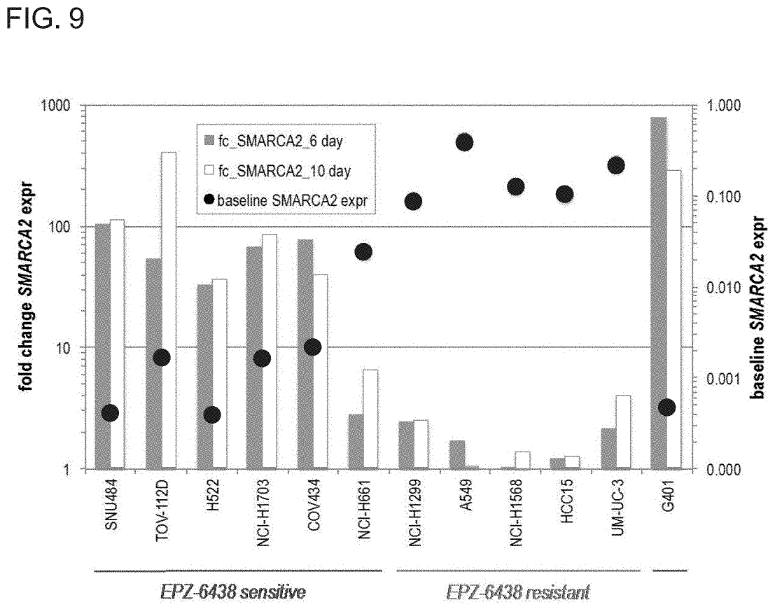

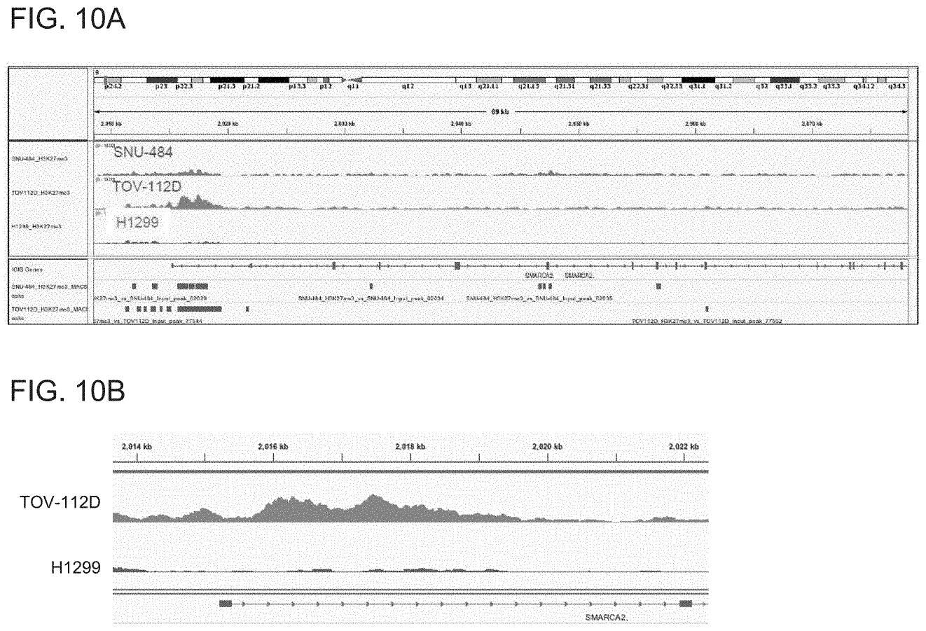

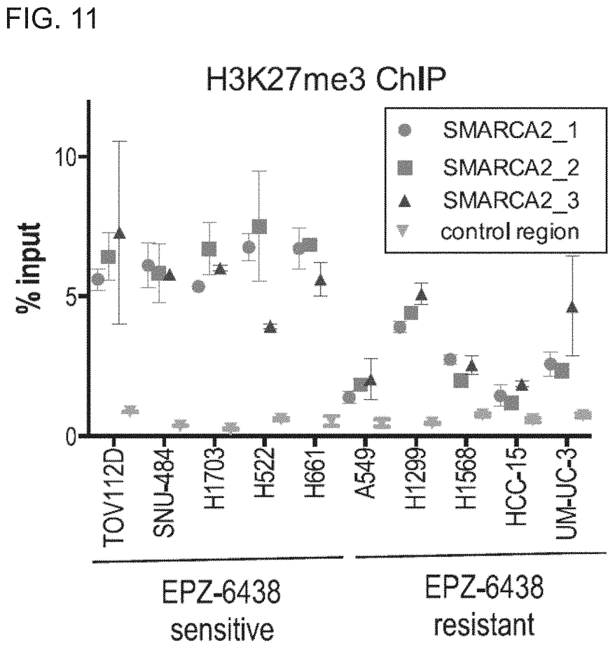

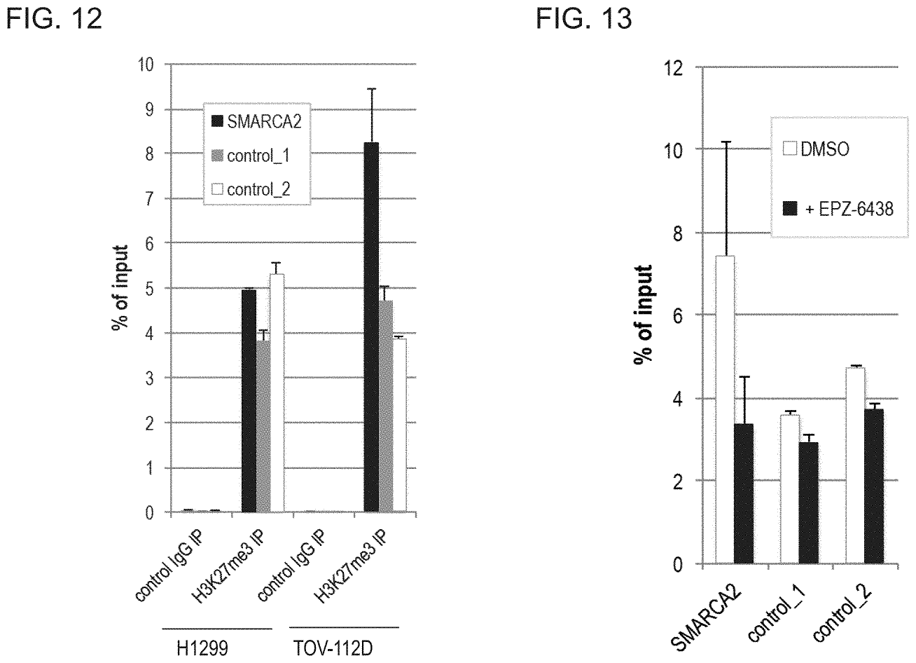

To elucidate differences underlying EPZ-6438 sensitivity, gene expression profiling was carried out across the 11 SMARCA4-mutant models. A supervised analysis of the most differentially expressed genes revealed that EPZ-6438-sensitive models exhibited a greater number of commonly repressed genes (FIG. 7). Among the genes that were upregulated, expression levels of the paralog SWI/SNF helicase, SMARCA2, were reduced in all SMARCA4-mutant models that were sensitive to EZH2 inhibition. To confirm these results, protein expression levels of several core SWI/SNF complex members were examined by western blot amongst the panel of SMARCA4-mutant cancer cell lines. Whereas most SWI/SNF components were expressed to an equal extent amongst the EPZ-6438-sensitive and EPZ-6438-resistant cell lines, a striking association of SMARCA2 repression with EPZ-6438 sensitivity was observed (FIG. 8). This repression of SMARCA2 was additionally observed at the level of the SMARCA2 mRNA transcript by quantitative RT-PCR (FIG. 9). Analysis of associated genomic data did not reveal copy number loss or mutations in SMARCA2 associated with the loss of SMARCA2 in this subset of cells. In addition, treatment with the DNA methyltransferase inhibitor, 5-aza-2′-deoxycytidine (5-aza), did not impact SMARCA2 mRNA levels, indicating that DNA methylation was not a cause for the repression of SMARCA2. To determine if SMARCA2 may be under EZH2-mediated suppression, cells were treated with EPZ-6438 prior to examining SMARCA2 mRNA levels by quantitative RT-PCR. Inhibition of EZH2 resulted in a strong induction of SMARCA2 transcript and protein in EPZ-6438-sensitive, but not EPZ-6438-resistant, cell lines (FIG. 9). To determine if SMARCA2 was directly suppressed by EZH2, H3K27me3 ChIP-seq was carried out in an EPZ-6438-sensitive model (TOV-112D) and an EPZ-6438-resistant (H1299) model. ChIP-seq analysis revealed that the SMARCA2 promoter was bound by H3K27me3 in EPZ-6438-sensitive TOV-112D cells, but not in EPZ-6438-resistant H1299 cells (FIGS. 10A and 10B). H3K27me3 occupancy was confirmed by ChIP-PCR across a full panel of SMARCA4-mutant cell lines at three targeted locations within the SMARCA2 promoter via PCR (FIG. 11). EPZ-6438 treatment resulted in a significant decrease in the association of H3K27me3 with the SMARCA2 gene promoter in TOV-112D cells (FIGS. 12 and 13). Taken together, these data indicate that EZH2 mediated the direct repression of SMARCA2.

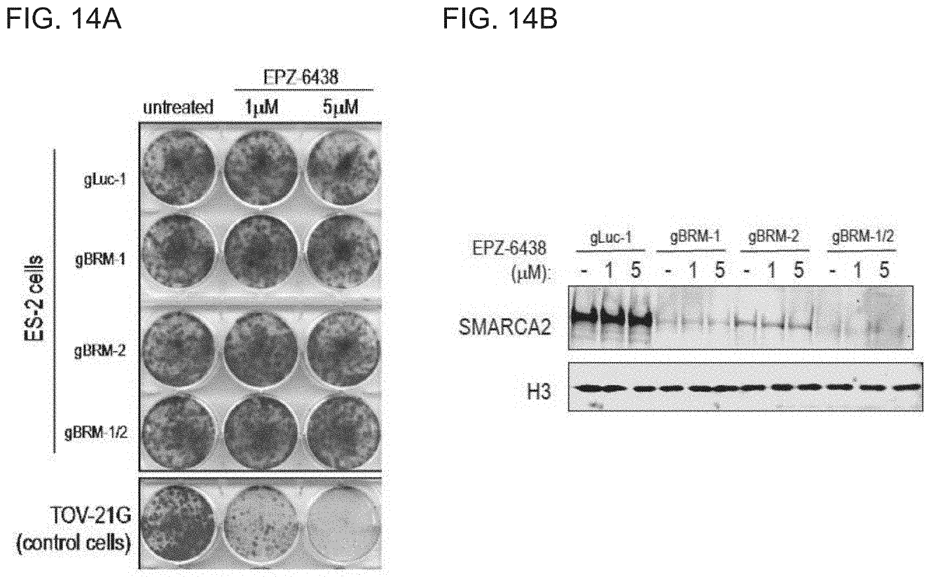

To test whether basally repressed SMARCA2 causes EZH2 inhibitor sensitivity, SMARCA2 (BRM1) was deleted in a wildtype model. Forced knockout of SMARCA2 did not lead to EZH2 inhibitor sensitivity, indicating that low expression of SMARCA2 was not the cause of EZH2 inhibitor sensitivity in wildtype cells (FIGS. 14A and 14B).

### Example 5. Assessment of the Ability of SMARCA2 to Compensate for SMARCA4 Transcription

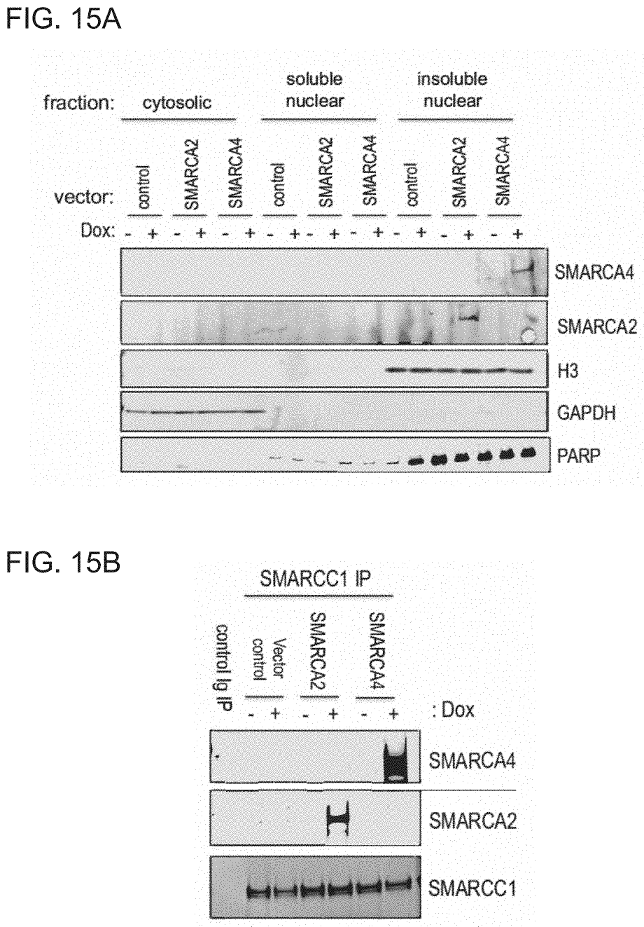

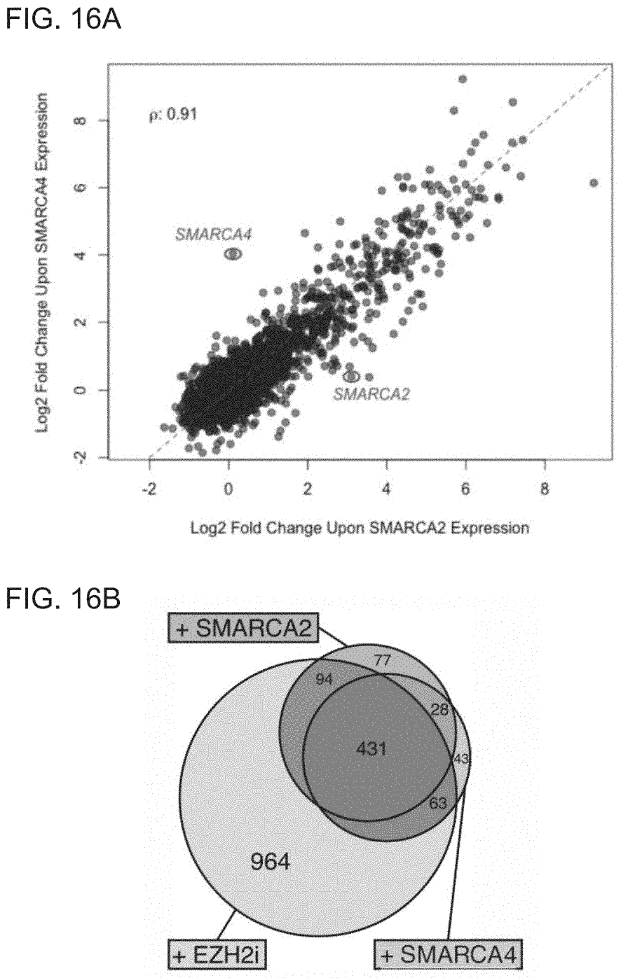

To address whether SMARCA2 could compensate for the transcriptional effects of SMARCA4 in this cellular context, TOV-112D cells were engineered to express either a doxycycline (dox)-inducible SMARCA2 or SMARCA4 construct. Doxycycline treatment of these cells resulted in the induction of SMARCA2 or SMARCA4 protein, localizing to the insoluble nuclear fraction and re-associating with the core SWI/SNF complex protein, SMARCC1 (FIGS. 15A and 15B). Analysis of gene expression changes following the dox-induced expression of SMARCA2 and SMARCA4 revealed a statistically-significant overlap in genes regulated by these helicases (FIG. 16A; P<2e-16, Fishers Exact Test). The induction of SMARCA2 and SMARCA4 resulted in the upregulation of gene expression, with over 70% of the most strongly induced genes shared between SMARCA2 and SMARCA4 (log 2 fold change 2). These genes significantly overlapped with genes that were derepressed upon EZH2 inhibitor treatment (FIG. 16B; P<2e-16, Fishers Exact Test).

### Example 6. Assessment of the Relationship Between SMARCA2 and EZH2 in EPZ-6438-Sensitive Cells

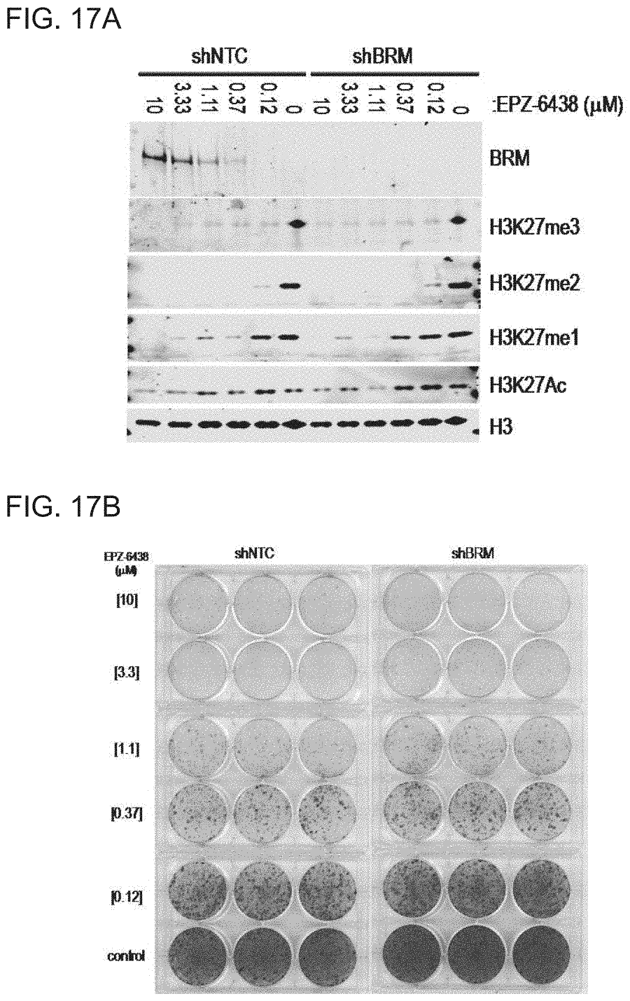

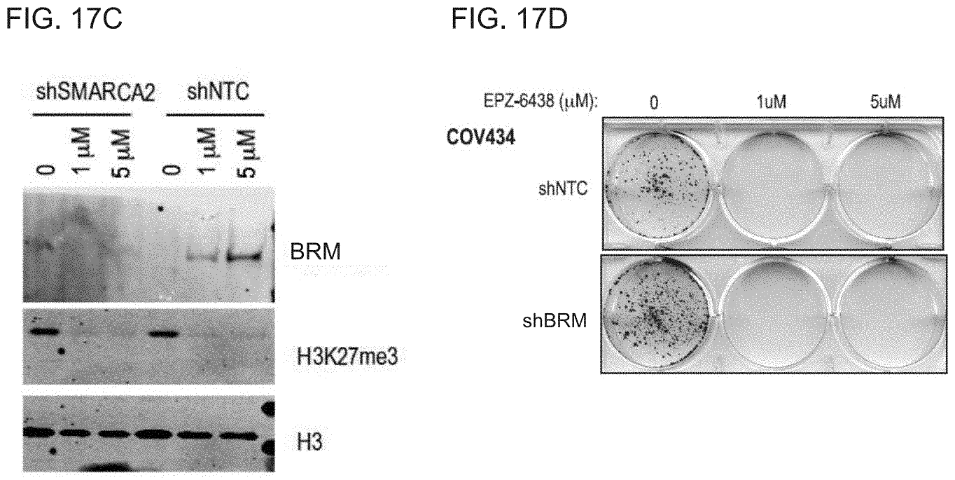

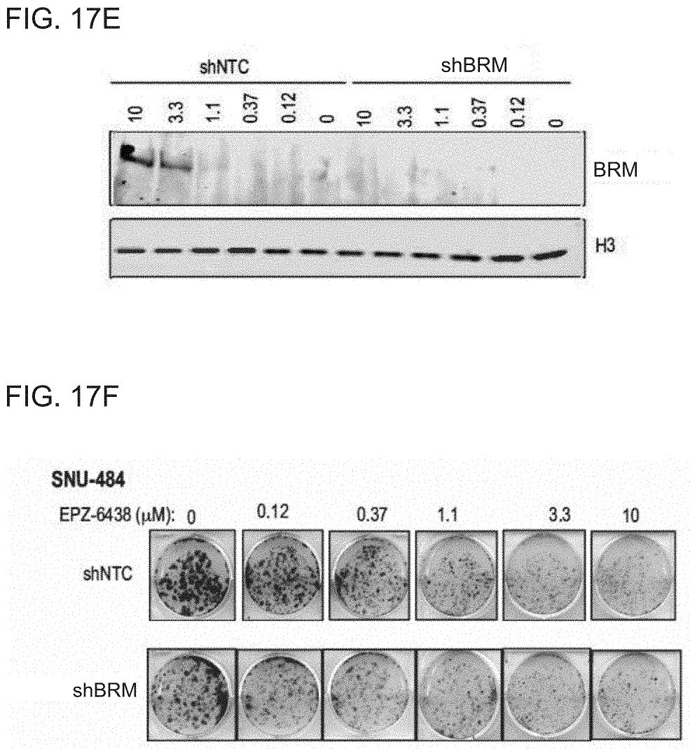

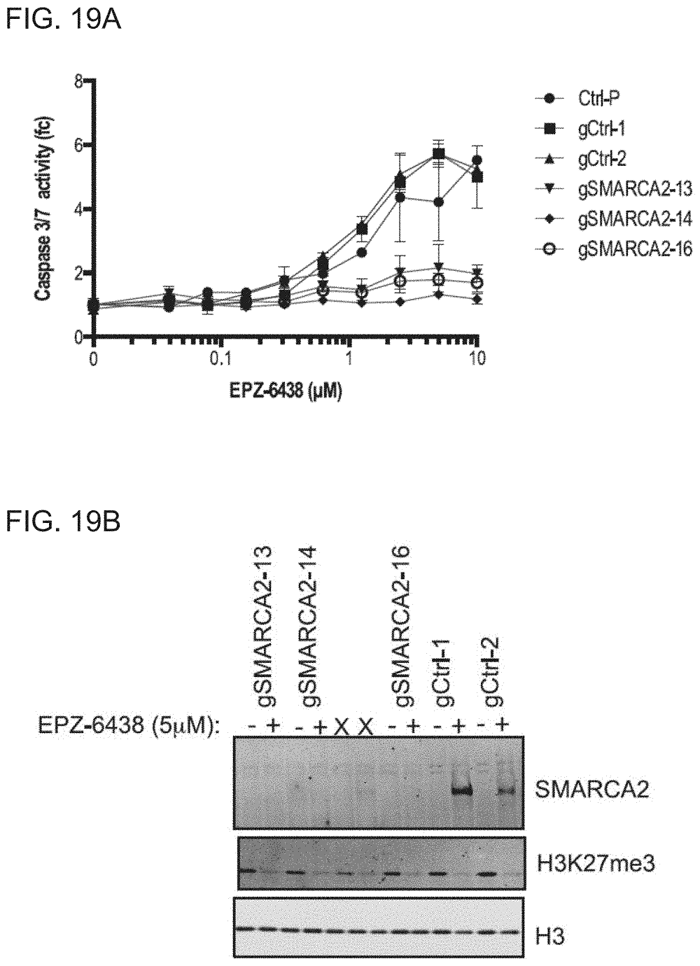

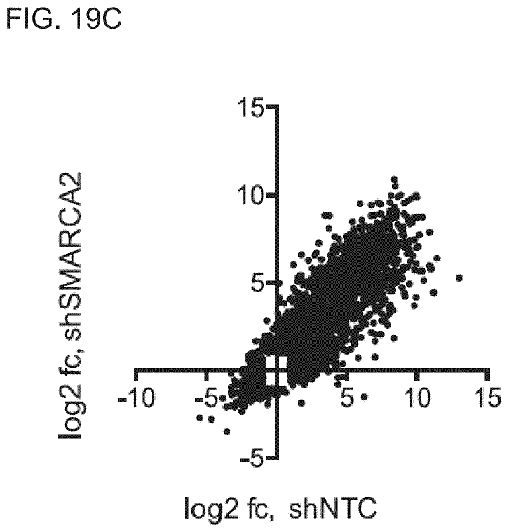

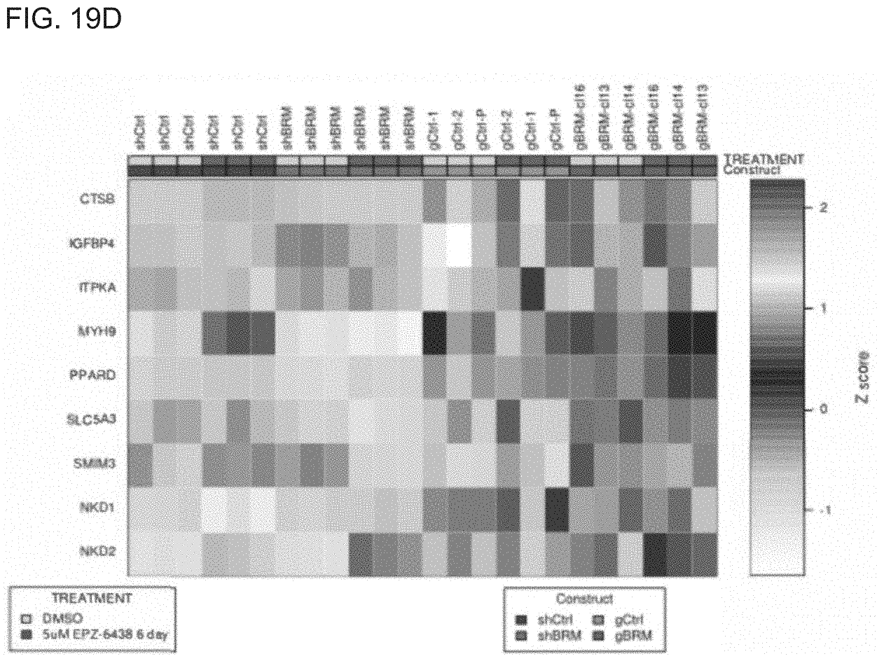

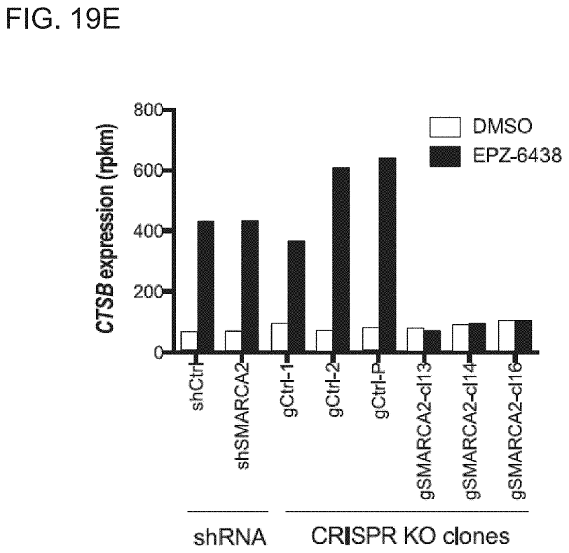

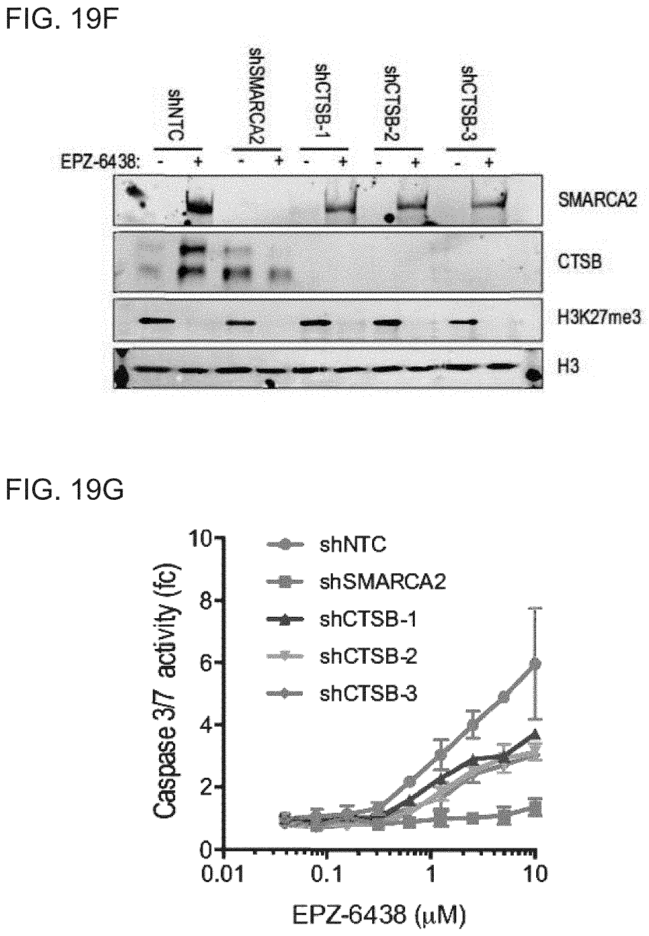

To determine if the derepression of SMARCA2 upon EZH2 inhibition was necessary for mediating the phenotypic effects of EPZ-6438 in sensitive models, shRNA targeting SMARCA2 was expressed in cells to specifically prevent induction of SMARCA2. As shown in FIGS. 17A-17F, shBRM, but not a non-targeting control (shNTC), abrogated the dose-dependent induction of SMARCA2 (BRM) in COV434 cells, SNU-484 cells, and G401 cells, but had no effect on the ability of EPZ-6438 to inhibit H3K27 methylation. Importantly, SMARCA2 shRNA did not affect the ability of EPZ-6438 to inhibit colony formation. A similar result was obtained in the SMARCA4-mutant cell line, NCI-H661, suggesting that the depression of SMARCA2 alone was not generally required for the growth defect upon EZH2 inhibition. However, in the SMARCA4-mutant model, TOV-112D, which undergoes apoptosis in response to EZH2 inhibition, expression of shBRM prevented the dose-dependent inhibition of colony formation (FIGS. 18A and 18B), as well as the dose-dependent induction of apoptosis in these cells (FIG. 18C). TOV-112D cells represented the only model tested that exhibited an apoptotic response to EPZ-6438, suggesting that the derepression of SMARCA2 may be necessary for this specific phenotypic response to EZH2 inhibition. This finding was confirmed in TOV-112D cells engineered to ablate the SMARCA2 gene by CRISPR-mediated genome editing (FIGS. 19A and 19B). To elucidate the mechanism(s) by which the EPZ-6438-mediated derepression of SMARCA2 contributes to apoptosis, gene expression changes regulated by EZH2 inhibition were evaluated in the presence or absence of shBRM expression, as well as in SMARCA2 KO clones. EZH2 inhibition resulted in a strong upregulation of gene expression in control cells, but blocking the induction of SMARCA2 had little effect on the overall number or magnitude of EPZ-6438-regulated genes, globally (FIGS. 19C and 19D). A small number of genes that were specifically impacted by both shSMARCA2 and SMARCA2 gene ablation were identified, including cathepsin B (CTSB). CTSB transcript and protein were strongly upregulated in control cells upon EZH2 inhibition, and this upregulation was blocked by targeting SMARCA2 (FIGS. 19E and 19F). To determine if CTSB can contribute to apoptosis in response to EZH2 inhibition in TOV-112D cells, three separate shRNAs targeting CTSB were expressed. Expression of shCTSB significantly suppressed the activation of caspase 3/7 in response to EPZ-6438 (FIG. 19G). As opposed to blocking the induction of SMARCA2 directly, blocking CTSB induction did not completely abrogate caspase 3/7 activation, suggesting that CTSB can contribute to apoptosis in response to EZH2 inhibition, but may not be fully sufficient for mediating apoptosis.

### Example 7. Assessment of the Role of Other SWI/SNF Complex Mutations in Inhibitor of H3K27 Methylation Sensitivity

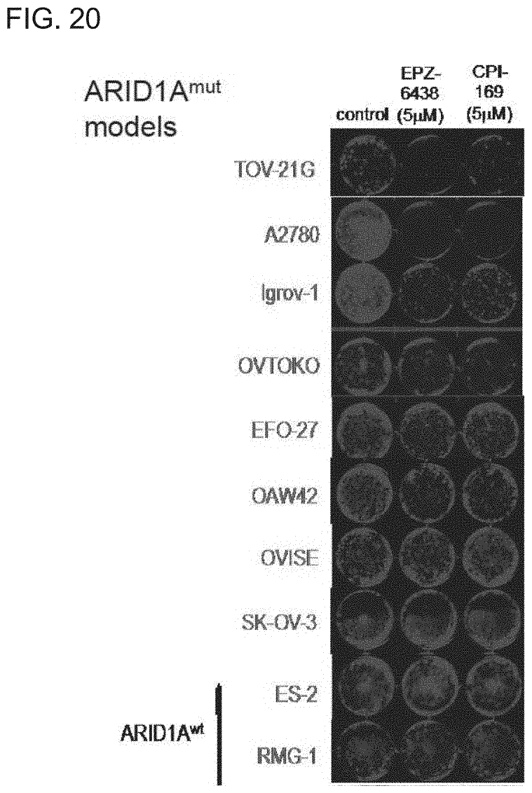

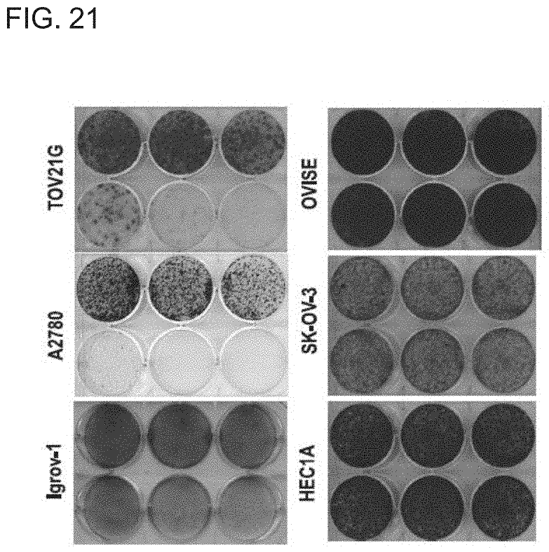

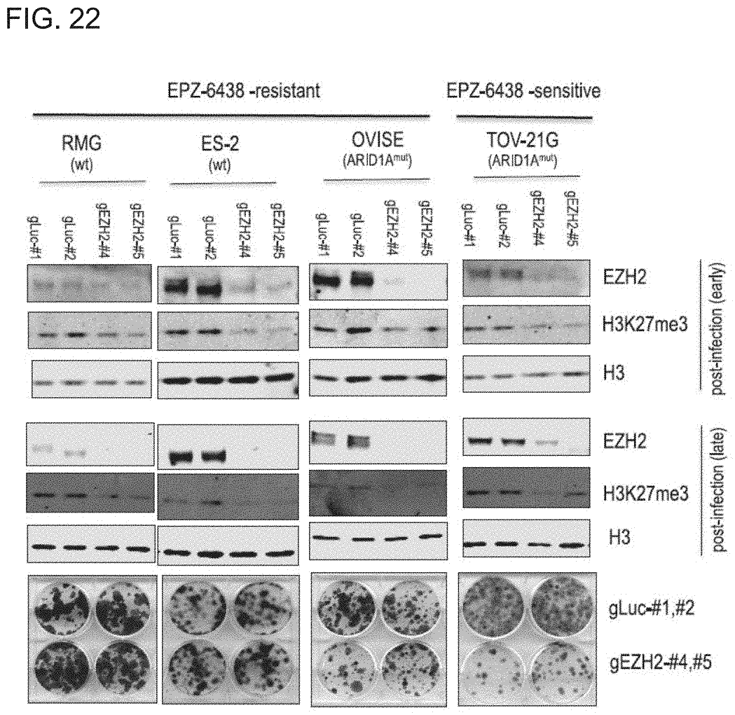

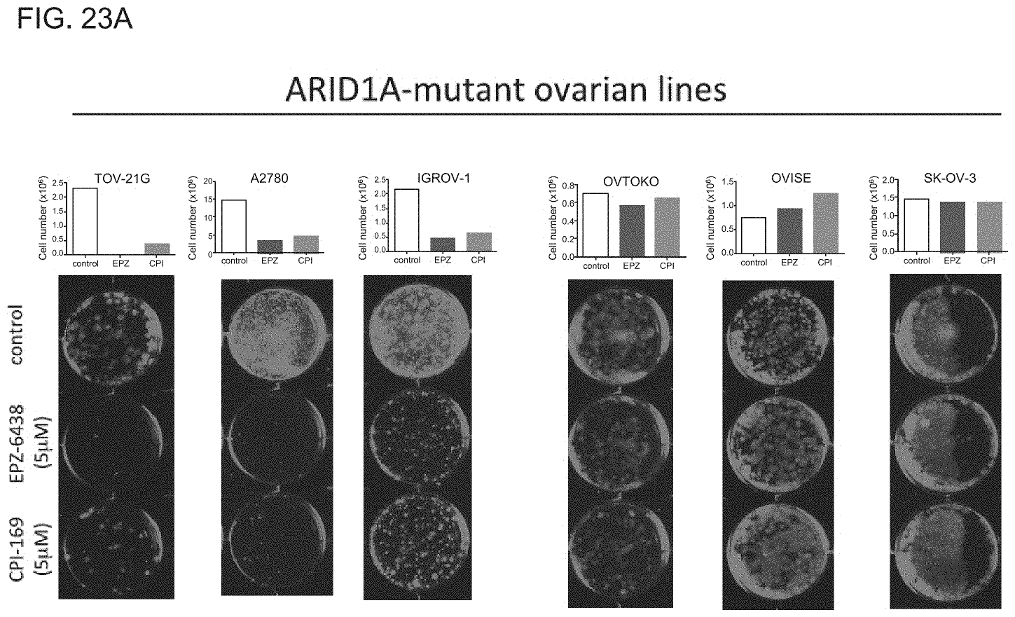

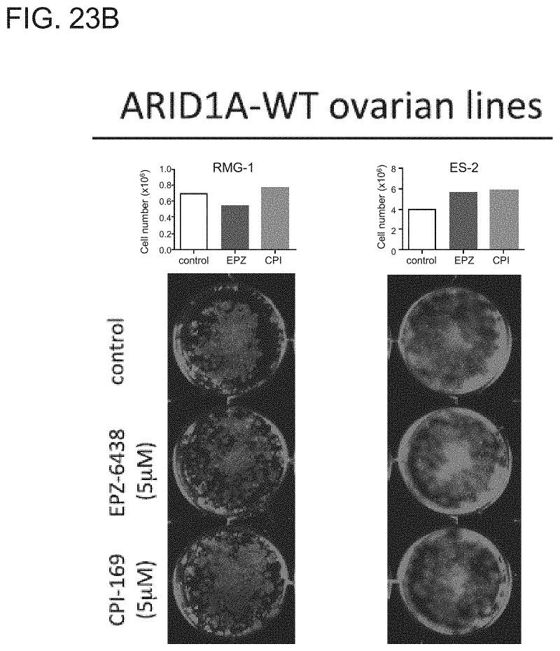

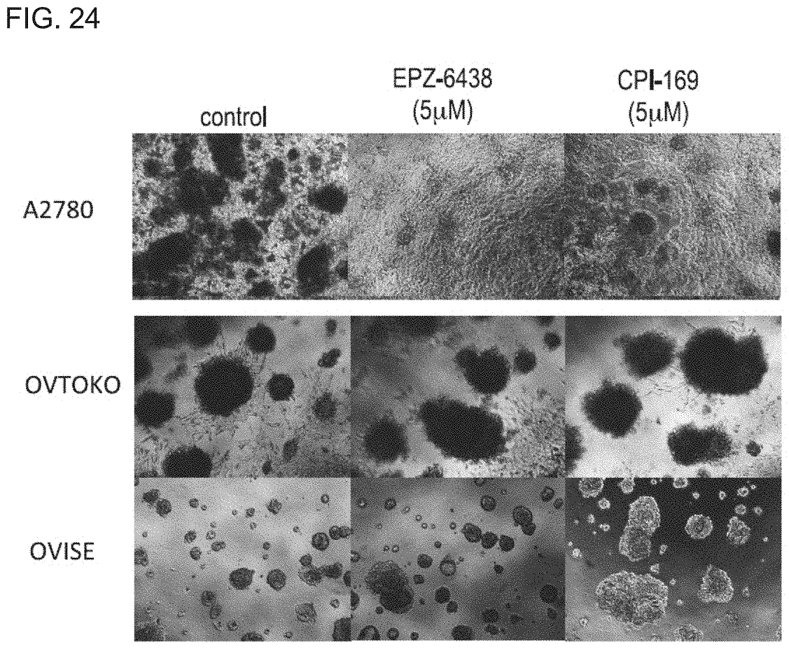

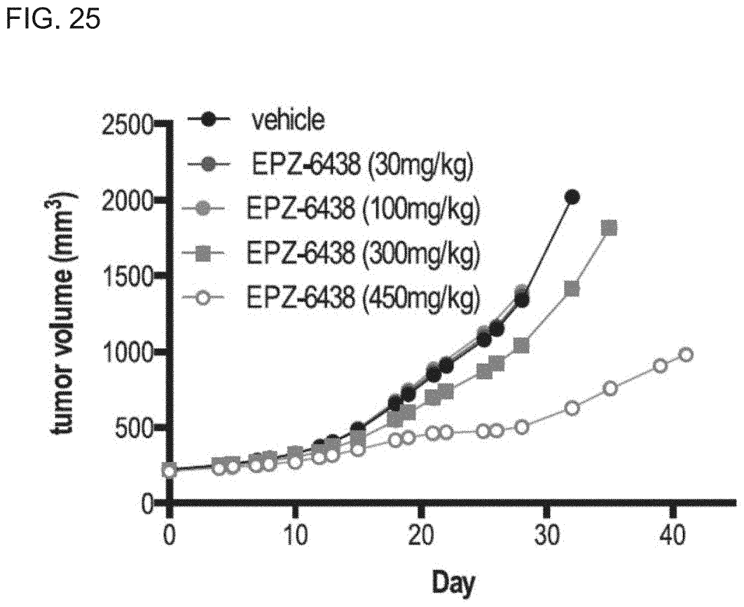

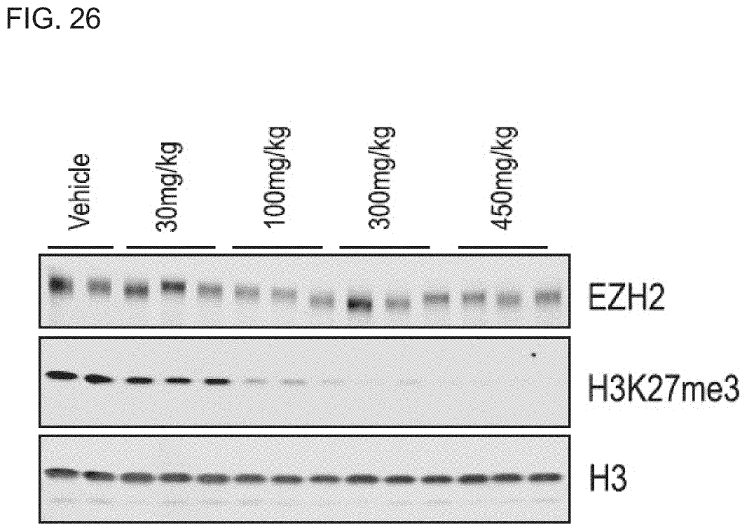

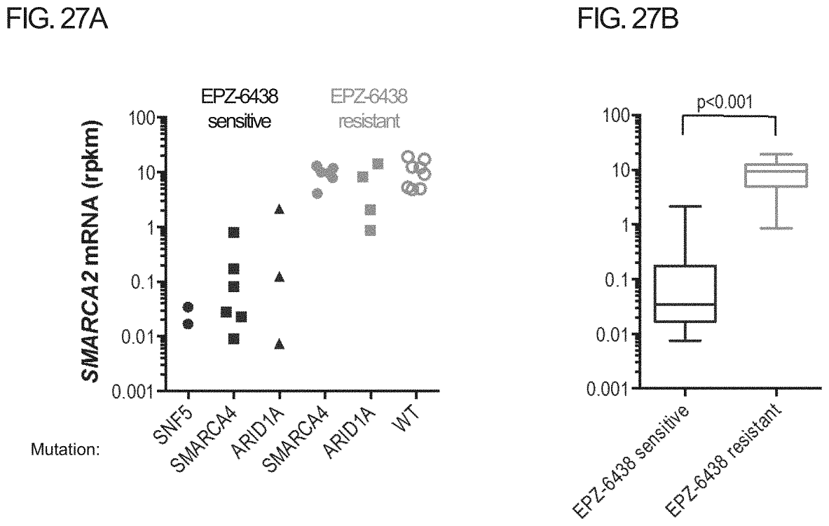

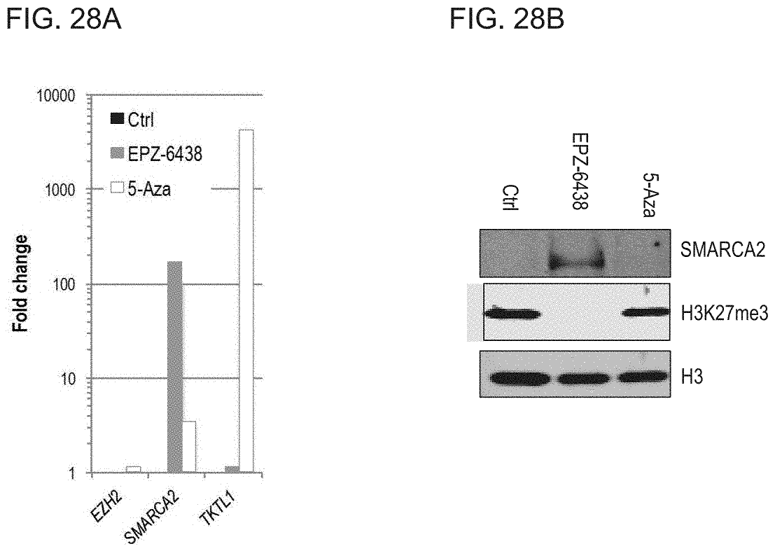

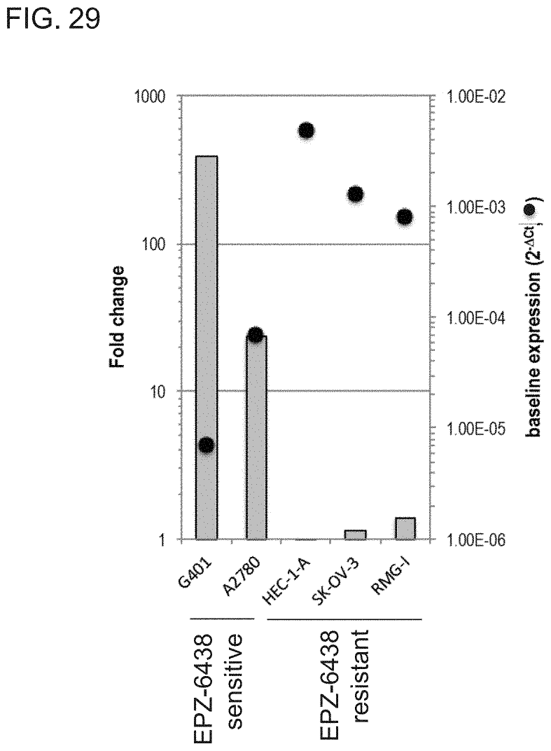

Similar to the observations in SMARCA4-mutant cancer cell lines, inhibition in colony formation was observed in a subset of ARID1A-mutant cancer cell lines (FIGS. 20 and 21), as well as in two SMARCB1-mutant malignant rhabdoid tumor lines. Growth inhibition was dependent upon EZH2, as genetic ablation of EZH2 inhibited clonogenic growth in the EPZ-6438-sensitive model, TOV-21G. Genetic ablation of EZH2 had no effect on colony formation in the EPZ-6438-resistant, ARID1A-mutant model, OVISE, or in control models harboring no known mutations in any SWI/SNF complex members (FIG. 22). The differential sensitivity to EZH2 inhibition was additionally phenocopied using another EZH2 inhibitor (CPI-169; FIG. 23) and by growing ARID1A-mutant cells in 3D cultures using Matrigel (FIG. 24). The observed in vitro activity further translated to in vivo efficacy, as treatment of SCID mice bearing TOV-21G tumor xenografts resulted in tumor growth inhibition at a dose of 450 mg/kg BID (FIGS. 25 and 26). Analysis of constitutive SMARCA2 transcript levels revealed that SMARCA2 was repressed in the SMARCB1-mutant and ARID1A-mutant cancer cell lines that were sensitive to EPZ-6438 (FIGS. 27A and 27B). No effect of EPZ-6438 on colony formation or repression of SMARCA2 was observed in a panel of cell lines that were wildtype for SWI/SNF complex genes. Treatment of the SMARCB1-mutant MRT line G401 with EPZ-6438, but not with 5-aza-2′-deoxycytidine, resulted in an induction in SMARCA2 levels (FIGS. 28A and 28B). In the context of ARID1A-mutant cell lines, EPZ-6438 resulted in an induction of SMARCA2 in EPZ-6438-sensitive A2780 cells, but not in EPZ-6438-resistant HEC1A or SK-OV-3 cells (FIG. 29). These data indicate that EZH2-mediated repression of SMARCA2 is also predictive of sensitivity to EZH2 inhibition in the context of SMARCB1 and ARID1A-mutant cancers.

### OTHER EMBODIMENTS

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. A method of identifying a patient having a cancer who may benefit from treatment comprising one or more inhibitors of histone 3 lysine 27 (H3K27) methylation, the method comprising determining an expression level of SMARCA2 in a sample obtained from the patient, wherein a decreased expression level of SMARCA2 in the sample as compared to a reference expression level identifies the patient as one who may benefit from treatment comprising one or more inhibitors of H3K27 methylation.

2. A method of optimizing therapeutic efficacy for treatment of a patient having a cancer, the method comprising determining an expression level of SMARCA2 in a sample obtained from the patient, wherein a decreased expression level of SMARCA2 in a sample as compared to a reference expression level indicates that the patient has an increased likelihood of benefiting from treatment comprising one or more inhibitors of H3K27 methylation.

3. A method of predicting responsiveness of a patient having a cancer to treatment comprising one or more inhibitors of H3K27 methylation, the method comprising determining an expression level of SMARCA2 in a sample obtained from the patient, wherein a decreased expression level of SMARCA2 in the sample as compared to a reference expression level indicates that the patient has an increased likelihood of benefiting from treatment comprising one or more inhibitors of H3K27 methylation.

4. A method of selecting a treatment for a patient having a cancer, the method comprising determining an expression level of SMARCA2 in a sample obtained from the patient, wherein a decreased expression level of SMARCA2 in the sample as compared to a reference expression level indicates that the patient has an increased likelihood of benefiting from treatment comprising one or more inhibitors of H3K27 methylation.

5. The method of any one of claims 1-4, wherein the expression level of SMARCA2 in a sample obtained from a patient is decreased by at least about 10% relative to the reference level.

6. The method of claim 5, wherein the expression level of SMARCA2 in a sample obtained from a patient is decreased by at least about 25% relative to the reference level.

7. The method of claim 6, wherein the expression level of SMARCA2 in a sample obtained from a patient is decreased by at least about 50% relative to the reference level.

8. The method of claim 7, wherein the expression level of SMARCA2 in a sample obtained from a patient is decreased by at least about 75% relative to the reference level.

9. The method of claim 8, wherein the expression level of SMARCA2 in a sample obtained from a patient is decreased by at least about 90% relative to the reference level.

10. The method of any one of claims 1-9, wherein the expression level of SMARCA2 is a median expression level.

11. The method of any one of claims 1-9, wherein the expression level of SMARCA2 is a mean expression level.

12. The method of any one of claims 1-11, wherein the reference expression level is selected from the group consisting of (i) the expression level of SMARCA2 in a sample obtained from the patient at a previous time point; (ii) the expression level of SMARCA2 in a reference population; or (iii) a pre-assigned expression level for SMARCA2.

13. The method of any one of claims 1-12, wherein the reference expression level of SMARCA2 is a median expression level.

14. The method of any one of claims 1-12, wherein the reference expression level of SMARCA2 is a mean expression level.

15. The method of any one of claims 1-14, wherein the expression level is an mRNA expression level.

16. The method of claim 15, wherein the mRNA expression level is determined by RNA-Seq, PCR, qPCR, RT-PCR, in situ hybridization, gene expression profiling, serial analysis of gene expression, or microarray analysis.

17. The method of claim 16, wherein the mRNA expression level is determined by qPCR.

18. The method of claim 16, wherein the mRNA expression level is determined by RNA-Seq.

19. The method of any one of claims 1-14, wherein the expression level is a protein expression level.

20. The method of claim 19, wherein the protein expression level is determined using a method selected from the group consisting of immunohistochemistry (IHC), immunofluorescence, mass spectrometry, flow cytometry, and Western blot.

21. The method of claim 20, wherein the protein expression level is determined by IHC.

22. The method of any one of claims 1-21, wherein the expression level of SMARCA2 in a sample obtained from the patient is decreased relative to the reference level and the method further comprises administering to the patient a therapeutically effective amount of one or more inhibitors of H3K27 methylation.

23. The method of claim 22, wherein the administering of the one or more inhibitors of H3K27 methylation is after the determining of the expression level of SMARCA2.

24. The method of claim 22, wherein the administering of the one or more inhibitors of H3K27 methylation is before the determining of the expression level of SMARCA2.

25. A method of treating a patient having a cancer, the method comprising administering to the patient a therapeutically effective amount of one or more inhibitors of H3K27 methylation, wherein the expression level of SMARCA2 in a sample obtained from the patient has been determined to be decreased as compared to a reference expression level.

26. The method of any one of claims 1-25, further comprising determining an occupancy level of H3K27 at a SMARCA2 promoter in a sample obtained from the patient.

27. A method of identifying a patient having a cancer who may benefit from treatment comprising one or more inhibitors of H3K27 methylation, the method comprising determining an occupancy level of H3K27 at a SMARCA2 promoter in a sample obtained from the patient, wherein an increased occupancy level of H3K27 at the SMARCA2 promoter as compared to a reference occupancy level identifies the patient as one who may benefit from treatment comprising one or more inhibitors of H3K27 methylation.

28. A method of optimizing therapeutic efficacy for treatment of a patient having a cancer, the method comprising determining an occupancy level of H3K27 at a SMARCA2 promoter in a sample obtained from the patient, wherein an increased occupancy level of H3K27 at the SMARCA2 promoter as compared to a reference occupancy level indicates that the patient has an increased likelihood of benefiting from treatment comprising one or more inhibitors of H3K27 methylation.

29. A method of predicting responsiveness of a patient having a cancer to treatment comprising one or more inhibitors of H3K27 methylation, the method comprising determining an occupancy level of H3K27 at a SMARCA2 promoter in a sample obtained from the patient, wherein an increased occupancy level of H3K27 at the SMARCA2 promoter as compared to a reference occupancy level indicates that the patient has an increased likelihood of benefiting from treatment comprising one or more inhibitors of H3K27 methylation.

30. A method of selecting a treatment for a patient having a cancer, the method comprising determining an occupancy level of H3K27 at a SMARCA2 promoter in a sample obtained from the patient, wherein an increased occupancy level of H3K27 at the SMARCA2 promoter as compared to a reference occupancy level indicates that the patient has an increased likelihood of benefiting from treatment comprising one or more inhibitors of H3K27 methylation.

31. The method of any one of claims 26-30, wherein the occupancy level of H3K27 in a sample obtained from a patient is increased by at least about 10% relative to the reference occupancy level.

32. The method of claim 31, wherein the occupancy level of H3K27 in a sample obtained from a patient is increased by at least about 50% relative to the reference occupancy level.

33. The method of claim 32, wherein the occupancy level of H3K27 in a sample obtained from a patient is increased by at least about 100% relative to the reference occupancy level.

34. The method of claim 33, wherein the occupancy level of H3K27 in a sample obtained from a patient is increased by at least about 500% relative to the reference occupancy level.

35. The method claim 34, wherein the occupancy level of H3K27 in a sample obtained from a patient is increased by at least about 1,000% relative to the reference occupancy level.

36. The method of any one of claims 26-35, wherein the occupancy level of H3K27 at the SMARCA2 promoter is a median expression level.

37. The method of any one of claims 26-35, wherein the occupancy level of H3K27 at the SMARCA2 promoter is a mean expression level.

38. The method of any one of claims 26-37, wherein the reference occupancy level is selected from the group consisting of (i) an occupancy level of H3K27 at a SMARCA2 promoter in a sample obtained from the patient at a previous time point; (ii) an occupancy level of H3K27 at a SMARCA2 promoter in a reference population; or (iii) a pre-assigned occupancy level of H3K27 at a SMARCA2 promoter.

39. The method of any one of claims 26-38, wherein the reference occupancy level of H3K27 at the SMARCA2 promoter is a median expression level.

40. The method of any one of claims 26-38, wherein the reference occupancy level of H3K27 at the SMARCA2 promoter is a mean expression level.

41. The method of any one of claims 26-40, wherein the reference occupancy level of H3K27 at the SMARCA2 promoter is determined by ChIP-seq or ChIP-PCR.

42. The method of any one of claims 26-41, wherein the occupancy level of H3K27 at the SMARCA2 promoter is increased relative to the reference occupancy level and the method further comprises administering to the patient a therapeutically effective amount of one or more inhibitors of H3K27 methylation.

43. The method of claim 42, wherein the administering of the one or more inhibitors of H3K27 methylation is after the determining of the occupancy level of H3K27 at the SMARCA2 promoter.

44. The method of claim 42, wherein the administering of the one or more inhibitors of H3K27 methylation is before the determining of the occupancy level of H3K27 at the SMARCA2 promoter.

45. A method of treating a patient having a cancer, the method comprising administering to the patient a therapeutically effective amount of one or more inhibitors of H3K27 methylation, wherein the occupancy level of H3K27 at the SMARCA2 promoter in a sample obtained from the patient has been determined to be increased as compared to a reference occupancy level.

46. The method of any one of claim 27-45, further comprising determining an expression level of SMARCA2 in a sample obtained from the patient.

47. The method of any one of claims 1-46, further comprising identifying a mutation in one or more genes encoding a nucleosome remodeling protein.

48. The method of claim 47, wherein the nucleosome remodeling protein is a SWI/SNF family protein.

49. The method of claim 48, wherein the SWI/SNF family protein is BRG1, SNF5 (INI1), SWI/SNF complex 155 kDa subunit, SWI/SNF complex 170 kDa subunit, BAF, zipzap protein, or BAF180.

50. The method of claim 48 or 49, wherein the one or more genes encoding a SWI/SNF family protein are selected from the group consisting of SMARCA4, SMARCB1, SMARCC1, SMARCC2, ARID1A, ARID2, and PBRM1.

51. The method of any one of claims 1-50, wherein the sample obtained from the patient is a cell sample, a tissue sample, a whole blood sample, a plasma sample, or a serum sample.

52. The method of claim 51, wherein the cell sample is a tumor cell sample.

53. The method of claim 51, wherein the tissue sample is a tumor tissue sample.

54. The method of any one of claims 1-53, wherein the cancer comprises a mutation in one or more genes encoding a SWI/SNF family protein.

55. The method of claim 54, wherein the one or more genes encoding a SWI/SNF family protein are selected from the group consisting of SMARCA4, SMARCB1, SMARCC1, SMARCC2, ARID1A, ARID2, and PBRM1.

56. The method of claim 55, wherein the cancer comprises a mutation in one or more of SMARCA4, SMARCB1, or ARID1A.

57. The method of any one of claims 1-56, wherein the cancer is selected from the group consisting of an ovarian cancer, a lung cancer, a gastric cancer, a bladder cancer, a breast cancer, a skin cancer, a colorectal cancer, a stomach cancer, a lymphoid cancer, a cervical cancer, a peritoneal cancer, a pancreatic cancer, a glioblastoma, a liver cancer, a bladder cancer, a colon cancer, a rectal cancer, an endometrial cancer, a uterine cancer, a salivary gland cancer, a renal cancer, a prostate cancer, a vulval cancer, a thyroid cancer, an anal cancer, a penile cancer, and a head and neck cancer.

58. The method of claim 57, wherein the cancer is an ovarian cancer.

59. The method of claim 58, wherein the ovarian cancer is an ovarian clear cell carcinoma.

60. The method of claim 58, wherein the ovarian cancer is a small cell carcinoma of the ovary.

61. The method of claim 60, wherein the small cell carcinoma of the ovary is a small cell carcinoma of the ovary, hypercalcemic type.

62. The method of claim 57, wherein the cancer is a lung cancer.

63. The method of claim 57, wherein the cancer is a gastric cancer.

64. The method of claim 57, wherein the cancer is a bladder cancer.

65. The method of any one of claims 1-56, wherein the cancer is a rhabdoid cancer.

66. The method of claim 65, wherein the rhabdoid cancer is a renal cancer or a brain cancer.

67. The method of claim 65 or 66, wherein the rhabdoid cancer is a malignant rhabdoid cancer.

68. The method of claim 67, wherein the malignant rhabdoid cancer is a SMARCB1-mutant malignant rhabdoid cancer.

69. The method of any one of claims 1-68, wherein the one or more inhibitors of H3K27 methylation comprise an inhibitor of H3K27 trimethylation.

70. The method of any one of claims 1-69, wherein the inhibitor of H3K27 trimethylation is an EZH2 inhibitor.

71. The method of claim 70, wherein the EZH2 inhibitor is a small molecule.

72. The method of claim 71, wherein the EZH2 inhibitor is selected from the group consisting of EPZ-6438, CPI-169, CPI-1205, EPZ005687, GSK-126, GSK343, and GSK503.

73. The method of claim 72, wherein the EZH2 inhibitor is EPZ-6438.

74. The method of claim 72, wherein the EZH2 inhibitor is CPI-169.

75. The method of claim 72, wherein the EZH2 inhibitor is CPI-1205.

76. The method of any one of claims 1-75, wherein the one or more inhibitors of H3K27 methylation disrupt the formation or activity of polycomb repressive complex 2 (PRC2).

77. The method of claim 76, wherein the one or more inhibitors of H3K27 methylation comprise a SUZ12 antagonist, an EED antagonist, or a jumonji antagonist.

78. The method of any one of claims 1-77, the method comprising administering to the patient a first inhibitor of H3K27 methylation and a second inhibitor of H3K27 methylation.

79. The method of claim 78, wherein the first inhibitor of H3K27 methylation and the second inhibitor of H3K27 methylation are co-administered.

80. The method of claim 78, wherein the first inhibitor of H3K27 methylation and the second inhibitor of H3K27 methylation are sequentially administered.

81. The method of any one of claims 1-80, further comprising administering to the patient an additional therapeutic agent.

82. The method of claim 81, wherein the additional therapeutic agent is an anti-cancer agent.

83. The method of claim 81 or 82, wherein the additional therapeutic agent and the one or more inhibitors of H3K27 methylation are co-administered.

84. The method of claim 81 or 82, wherein the additional therapeutic agent and the one or more inhibitors of H3K27 methylation are sequentially administered.

85. The method of any one of claims 82-84, wherein the anti-cancer agent is selected from the group consisting of a chemotherapeutic agent, a growth inhibitory agent, a cytotoxic agent, an agent used in radiation therapy, an anti-angiogenesis agent, an apoptotic agent, an anti-tubulin agent, and an immunotherapy agent.

86. The method of claim 85, wherein the anti-cancer agent is a chemotherapeutic agent.

87. The method of any one of claims 1-86, wherein the patient is a human.

88. A composition comprising one or more inhibitors of H3K27 methylation for use in a method of treating a patient suffering from a cancer, wherein a sample obtained from the patient has been determined to have a decreased expression level of SMARCA2 in a sample as compared to a reference expression level.

89. A composition comprising one or more inhibitors of H3K27 methylation for use in a method of treating a patient suffering from a cancer, wherein a sample obtained from the patient has been determined to have an increased occupancy level of H3K27 at a SMARCA2 promoter in a sample as compared to a reference occupancy level.

90. The composition of claim 88 or 89, wherein the patient is a human.

91. A kit for identifying a patient who may benefit from treatment comprising one or more inhibitors of H3K27 methylation, the kit comprising:
(a) polypeptides or polynucleotides capable of determining an expression level of SMARCA2 in a sample; and
(b) instructions for using the polypeptides or polynucleotides to identify a patient that may benefit from treatment comprising one or more inhibitors of H3K27 methylation.

92. A kit for identifying a patient who may benefit from treatment comprising one or more inhibitors of H3K27 methylation, the kit comprising:
(a) reagents capable of determining an occupancy level of H3K27 at a SMARCA2 promoter in a sample; and
(b) instructions for using the reagents to identify a patient that may benefit from treatment comprising one or more inhibitors of H3K27 methylation.

93. The kit of claim 91 or 92, wherein the patient is a human patient.
